SEQUENCE LISTING (58 sequences)

SEQ ID NO: 1 - DNA, 2031 bp, Arabidopsis thaliana
atgattccttggggtggagttacttgctgcctcagcgccgctgctctttatcttctcggccggagtagtggcaggtttgtctgatctcttttatatttcatcttcccaaagagattatcaatcaatcaaatcctttcttatccttttgagtgcagggatgctgaagtactcgaaacagtcactagggttaatcagctcaaggagttaggtaatcttcttctcccctgattgcttcatctactctcaggatgaagttttgatcatgttttctgattgttctgtatgtgtagctcaattgctagaattagatagcaagattctgcctttcattgttgcggtatcaggaagagtcggctctgagacacctatcaaatgcgagcatagtggcatacgcggtgttattgttgaagaaacggtatgttgtagactgatgattagcgcatggaacttagtttgttttctggtttaatcgattaggttttatgtgaaactctagtaattgcatgatcttttcaggcggaacaacatttcctgaaacataatgagacgggttcttgggtacaagatagtgcgttgatgctatcaatgagcaaagaggttccttggtttctggtaagtctagtctagtagcatagtgtttgaaacaactgtgattggatgttttcttaataccatttccaaaactgtttgggataggacgatgggacaagccgtgtccatgtaatgggagctcgtggtgcgacgggttttgccttgactgttggtagtgaagtttttgaagagtcaggacgctcgcttgtacgaggaacacttgattatcttcaaggacttaaggtttttacttttctttccggttctttgtttgttggcttctctatttgcttaagcggccattgttttgtttcagatgcttggagttaagcgcattgagcgtgttcttccaactggaataccgctaacaattgttggtgaggtatgtcgtattctcagtgttttcgggtcctctcttttgcttaagttgtaactgttgatagagatacatagcacactaactccttcatcagtctggtatttgcctcttgaaattttctcaaagttcctttaatagcaatatttgtaggaagtgggattgatctatgtatagaggcttaccgatgagtttaaatctaatttgtgttgctgccatgtataacaggctgtcaaggacgatattggagaattcaggattcaaaaacctgacaggggccctttctacgtctcttctaaatcactcgatcagctcatttctaatttgggaaaatggtcaaggtcgtgtctctctcctctctcggttcttctcctatactcttgtagaaaaacggcaatgagccaaactgattgagaagagtataatttacaggttgtacaaatatgcctccatgggttttactgttcttggtgtgttcctaattacgaagcatgtcattgactctgttctagagagaagacggcggagacagttacaaaaaaggtatgtcacagatttgtctgtctaaaagtgaataaccgttctcaagcatgagtactagatcggcttgtttctctcgaaactatgtacacacaaaattaagtagtcagctgtttttgcagagtgcttgacgcagcagcaaagagagctgagctagagagtgaaggtatccattggtgaatctctttattctacatataggttgcactggctctgactacaatctcttctgaccaggttcaaacgggacacgtgagagcatttcagattctaccaagaaagaagacgctgttcctgatctctgtgtgatatgcctagagcaggagtacaacgctgtgtttgtcccgtaagcattcttccgccatttttggttgattctgcatttgcaacttgctaaaatgcttgtggttggtactcgcaggtgtggtcatatgtgctgctgcaccgcatgctcctcccacttgaccagctgtccactttgtcggagacgaatagatctggcggttaagacatatcgtcactga

SEQ ID NO: 2 - DNA, 1584 bp, Arabidopsis thaliana
atgagaatattgagagagatcgaagcaaaggatcattcaattccaaccctctgaatcttttaatttcccctttcgaaattctcctcttctttcactgcttctagtttctaattcttcaaactcttcctcgattcatactcataactctcattagctaatttcgcatgatcttcttccatctctctgtgttctaaatccagattcgtttcactcccatctctatttcattcaattcgctgcatccagattcaaaacctacctctatctctctgctcatcaataacttcaaaggtattgttgttcttctgcaaacaagtaagagtgacttcagagtctgatgattccttggggtggagttacttgctgcctcagcgccgctgctctttatcttctcggccggagtagtggcagggatgctgaagtactcgaaacagtcactagggttaatcagctcaaggagttagctcaattgctagaattagatagcaagattctgcctttcattgttgcggtatcaggaagagtcggctctgagacacctatcaaatgcgagcatagtggcatacgcggtgttattgttgaagaaacggcggaacaacatttcctgaaacataatgagacgggttcttgggtacaagatagtgcgttgatgctatcaatgagcaaagaggttccttggtttctggacgatgggacaagccgtgtccatgtaatgggagctcgtggtgcgacgggttttgccttgactgttggtagtgaagtttttgaagagtcaggacgctcgcttgtacgaggaacacttgattatcttcaaggacttaagatgcttggagttaagcgcattgagcgtgttcttccaactggaataccgctaacaattgttggtgaggctgtcaaggacgatattggagaattcaggattcaaaaacctgacaggggccctttctacgtctcttctaaatcactcgatcagctcatttctaatttgggaaaatggtcaaggttgtacaaatatgcctccatgggttttactgttcttggtgtgttcctaattacgaagcatgtcattgactctgttctagagagaagacggcggagacagttacaaaaaagagtgcttgacgcagcagcaaagagagctgagctagagagtgaaggttcaaacgggacacgtgagagcatttcagattctaccaagaaagaagacgctgttcctgatctctgtgtgatatgcctagagcaggagtacaacgctgtgtttgtcccgtgtggtcatatgtgctgctgcaccgcatgctcctcccacttgaccagctgtccactttgtcggagacgaatagatctggcggttaagacatatcgtcactgaacaacaactcaggcctcagaaacattctctacttgagtcttgtctgtaaataccgcaaaatcaaaacattacacagtttagcgttcgatattccctttggtttgatttcgacaacaaaacattttgaattatatagaaacataaggtgtttactcgatttgcaaaacagtacattcgtgtttacttattcgtgttgttgccaatgccatgaggtg

SEQ ID NO: 3 - protein, 343 aa, Arabidopsis thaliana
MIPWGGVTCCLSAAALYLLGRSSGRDAEVLETVTRVNQLKELAQLLELDSKILPFIVAVSGRVGSETPIKCEHSGIRGVIVEETAEQHFLKHNETGSWVQDSALMLSMSKEVPWFLDDGTSRVHVMGARGATGFALTVGSEVFEESGRSLVRGTLDYLQGLKMLGVKRIERVLPTGIPLTIVGEAVKDDIGEFRIQKPDRGPFYVSSKSLDQLISNLGKWSRLYKYASMGFTVLGVFLITKHVIDSVLERRRRRQLQKRVLDAAAKRAELESEGSNGTRESISDSTKKEDAVPDLCVICLEQEYNAVFVPCGHMCCCTACSSHLTSCPLCRRRIDLAVKTYRH

SEQ ID NO: 4 - protein, 48 aa, Arabidopsis thaliana
CVICLEQEYNAVFVPCGHMCCCTACSSHLTSCPLCRRRIDLAVKTYRH

SEQ ID NO: 5 - protein, 19 aa, Arabidopsis thaliana
MIPWGGVTCCLSAAALYLL

SEQ ID NO: 6 - protein, 21 aa, Arabidopsis thaliana
SRLYKYASMGFTVLGVFLITK

SEQ ID NO: 7 - DNA, 1824 bp, Arabidopsis thaliana
atgtcctcgccggagcgtgctctcttgaatctattgacggacatcgctctctccttcgacggcgcaatcctcggactgactttagctgttagcgccgtcggttctgctctcaagtatgcttctaccaacgccgcgttgaagaagatcaaagatgcacccgaagtctccatttccgacctccgatcactgcttccggcttctgaagacaaatcagagactaacgataacaggaagagcaatgatcagcgaatcgttgtggttcgcggagtcgtcaaaccgaaaatttccggtgatgagggttacaagaacaacaacgtgttaatctctccggagactggagataaagctcttatcattcagagaacgcaaactgtacgttgctctaaaccctaagtgtttatgtttccctcaaattgcaattgacattgacctaattttgctttgtgttcttatcaaaggcttaggttaaagagtttggatgatttcttcatgattaggtgttcattgtgattgattaagttaggattatgtaagttggtactggtgtgatctcagaagggaaatgtatgtttctaatgtcatcattttgttgttgacagtatgtgtatagtggatggaagagattgtttcagtccactggtcaccggtttatgttggagagatcattgcgcaaacacggtgctgattttacgagaacggtaaagtctctctatcagcttcagagctttcaactttggctctgtatccatgatcaaacttgttttgtgtatcctttgctttgtgcaggtcccatttgtcattgttggaaaggatcagcaatcgaactctagttttgtcgctgttaacatggatggctcaaggcaacctcttccgcttaccactgtgtataatcgcttgcagcctattaattcttcgtttcttcaagcctttttgtaccccgattatcctgtgagtatatatatattcaaagaaatttctggttttgaggaactgatggttacttgttcttggctaactttgtaatcctttctcttccaaggttggtctgcttgatatagagaagattttaccacctgggaaagatataacagctgttggcatctacagttttaataatggagttcctgaaatcaaatcgtgccaagatcttccttatttcttgtgagattttatctgaatatttttcgttcttatatatctgcctgcgttctagctcaaattattacttcatcttcttctttatgttttctcaatctgttgatcatttgacaagatttagtccattgttgcagaaaggctctatttgctgttcatatataaatctttatgaataatctttgcagatcagagatgaccaaggacaagatgattgaggaccttatggaacagaccaactttatattcttgggcagcgttatcttgggcattgtgtctgttggcatccttagctatgctgctgtcaggtactggagttgtgagagatcagatttggttttagatttgggcaatgtttcgatttgaaatctgtttaactgtcttatcttcaggacctggaataaatggaaacaatggaaccatcagagagaactgccacagagaccgaacgattctgtggtcgatgatgaacccgaagatgcagatgaaatcccagatggagaattgtgtgtgatctgcgtgtcaagaaggagagtacctgcgtttattccctgtggacatgtagtatgttgcaggcgatgtgcttcaaccgtggaacgagagttaaaccctaagtgtccagtttgtcttcagagcattaggggatctatgcgtgtatattactcttag

SEQ ID NO: 8 - DNA, 1386 bp, Arabidopsis thaliana
acttgtccgtgtgaccgtgtctacgacgttgaaatcgaaattaccacaatgtcctcgccggagcgtgctctcttgaatctattgacggacatcgctctctccttcgacggcgcaatcctcggactgactttagctgttagcgccgtcggttctgctctcaagtatgcttctaccaacgccgcgttgaagaagatcaaagatgcacccgaagtctccatttccgacctccgatcactgcttccggcttctgaagacaaatcagagactaacgataacaggaagagcaatgatcagcgaatcgttgtggttcgcggagtcgtcaaaccgaaaatttccggtgatgagggttacaagaacaacaacgtgttaatctctccggagactggagataaagctcttatcattcagagaacgcaaacttatgtgtatagtggatggaagagattgtttcagtccactggtcaccggtttatgttggagagatcattgcgcaaacacggtgctgattttacgagaacggtcccatttgtcattgttggaaaggatcagcaatcgaactctagttttgtcgctgttaacatggatggctcaaggcaacctcttccgcttaccactgtgtataatcgcttgcagcctattaattcttcgtttcttcaagcctttttgtaccccgattatcctgttggtctgcttgatatagagaagattttaccacctgggaaagatataacagctgttggcatctacagttttaataatggagttcctgaaatcaaatcgtgccaagatcttccttatttcttatcagagatgaccaaggacaagatgattgaggaccttatggaacagaccaactttatattcttgggcagcgttatcttgggcattgtgtctgttggcatccttagctatgctgctgtcaggacctggaataaatggaaacaatggaaccatcagagagaactgccacagagaccgaacgattctgtggtcgatgatgaacccgaagatgcagatgaaatcccagatggagaattgtgtgtgatctgcgtgtcaagaaggagagtacctgcgtttattccctgtggacatgtagtatgttgcaggcgatgtgcttcaaccgtggaacgagagttaaaccctaagtgtccagtttgtcttcagagcattaggggatctatgcgtgtatattactcttagtgaccctgaatcttctctcattgtatgtaatagggtaatgcaatttgtactctcagagaatcggaagttaacaaaatagtttgacaatgatttgaaagttcgtcaagtcactcaatatagagtgagagattgtagaggatatgaaagcaaagtttttaaaaggaagaaattttgtaaccgataaca

SEQ ID NO: 9 - protein, 383 aa, Arabidopsis thaliana
MSSPERALLNLLTDIALSFDGAILGLTLAVSAVGSALKYASTNAALKKIKDAPEVSISDLRSLLPASEDKSETNDNRKSNDQRIVVVRGVVKPKISGDEGYKNNNVLISPETGDKALIIQRTQTYVYSGWKRLFQSTGHRFMLERSLRKHGADFTRTVPFVIVGKDQQSNSSFVAVNMDGSRQPLPLTTVYNRLQPINSSFLQAFLYPDYPVGLLDIEKILPPGKDITAVGIYSFNNGVPEIKSCQDLPYFLSEMTKDKMIEDLMEQTNFIFLGSVILGIVSVGILSYAAVRTWNKWKQWNHQRELPQRPNDSVVDDEPEDADEIPDGELCVICVSRRRVPAFIPCGHVVCCRRCASTVERELNPKCPVCLQSIRGSMRVYYS

SEQ ID NO: 10 - DNA, 6940 bp, Solanum lycopersicum
atggttccatgggccggactctcttgctgtttgagtgcagctgctctttaccttctcggtaggagcagtggaaggtttgaacttctcttcttccaatttcattgctctactgtgtatatttatgtccatgaatttagatggatttgatttctaatacagagatgctgaagttcttaaatccgttacaagggttaatcaattgaaggatcttggtaagtaaatttgtaatttcatatggatggcttagtctctccgctaattactttcagcagacgtttcaattaccagtacaaggaggttgcgtcatatctttactgcatccatcgttcataagaaaaagaactaactttttatccattgggaaaagagcagatggatttttagttttagtgtgtgatatcatgctacacttgtaactcatttgaggtagtatgttcgaccatagggaaaatttctattttggcaccaaatgagagtcaggcagtgttagatgatatacttaggcgcatcagtcccatggtaagttcgagtaaattggggatactattcaaagtggatggaggaaaggtgtgtgaccatgcaaattgagtttcctcttttaactggatggatctccttttggtttctctacgagtagtagaggcattagacaaggatatcctcatcttcctgtgttccttttagttatgaaggtgctcagcgaattagtggagtagctgagaacatgaggctgttcatgatatttgaaagagggatatctgttagatcagaggcattacaaattctagaatatagaacttaatattggatagttgtatatatcttattgtaccttttgttagtgcattctctcaaccttgaattatctttgtactccttttattttagcacaactactagatactgcatccaaggtgttgcctctggtggttactatatctggaagggttggatcagatacaccaattaactgtgagtacagtggtctacgaggcgtgattgtggaagaaactgtatgttatctcgttctttaaataatagcataaaatgtttgttttcttttcaatgatgttttgttaaatgttcttgatattattacaggccgaacaacattttctgaaacacaatgatgcaggttcttggatacaggattctgccttgatgctctcaatgtgtaaagaagttccgtggtacctggttggaatgacttccttttgtctttttatcttaatagtcctgtaggctgtttaaatgaattcttaagaattctgctttactttgatgtatgaatttttaatagtttttgacttgtgtaccaggatgatggcacaggtcgcacttttattgttggtggccgtggtgccacgggtttggtactgacagttggaagtgaagccttcgaggaagcagggagatcatttgtgcgagggacattggattatcttcaaggtcttaaggtctgttttatcatataactgctgtttgttgcaggttgtaatttgtattttcataactcttgtgtctaaaaaaattttgaaggtaactttgactctgacctattattttcacaagtaggaggccaacgccatttttaattagcgtcgtaagtgtagaacatagtgaattgcaggaaaccactctcaccagtcactcacatatccgttcatgtatgaccatgcttttatgtattttggtgaagttcattaattgatgattacttttgaagatgcttggagtaaagaggattgaacgtgtgctgccagttggtactcctttgactgttgttggcgaggtatgatatgccttttgagcagaaaaagttgcaacattttcatagaagtaacacaaattaacagaattcagatgatacgagtagcaactctctgtcagcttattctttagtcgttcactatgtattctcaacactgatgtcataaaaaatgtattcctgacactaactgtaaatagaattgatgacagctaagaagaatgatagtgaaacaaaacaccttctatatacttaattaattctcaacaccttctatatacttaattacttctcaacaccttctatatagttctttattttctgtaaggtaactcttagtgtagtttccagagatctttgttgcatcacgttattgtggttattggaaggaaagaagaacaagaacaattacaagaggtgacttccagagaggattcaacaatagaacaaactcaaaatgatccaagagttgcatatatttaactaactggttaaatggtgcacgtccttgaattacataaaatacatatcaagacctatggacatcctcaatatatataaaatgattcagttgttttgtcacagtcgagtggaagcaagcatgcagagtttagactttcttttaatgtgtcattccttgtgttcttcatggtcatcatgacctgattggtagagaatagttttaaagggaaataataaaaatagcaaatagattatagaaatagctatcaaggaaataagaggatcaaaatgcatccaatattacaagtatactttagttatacttgagcattcattgttcacttctactacattactctgcttgaacactagttgacccttctctgtgtgcgccaagggggctggatgcatataagagctgtctgtatattgtgtatcaacttcattttcctttgtgattgtatcaggctgtcaaagatgacattgggacagttcggatccagcgaccacacaaaggcccattttatatctctcataaaactattgaccagctcattgcaaatcttgggagatgggcaaggttgatatgctgtactttcgaactgtaatcaaccctatattcccctactaattttacatattttttcacatgcccatatccaatcttatgttcaagctcatagacatgagtacgttctacttcaattaggttggtagtgtactttgtagggtctgtatcaagtaccctttccactatcatatctgttcaacaaagatatgtctagaaaaataaaagttcatgaattgacttgcatgaacatatctattcccccatgagtatgtcaaggcgcccccaggatttgtttgttttagcctcgtacatagtattgataagttcttttactttcaggtggtacaagtatgcttcaatgggttttactgctgtcggtgtatatctacttgtgaagcatacttttcattatatgatggaaagaaagcgtcactgggaattgcgcaggaggtatatttaggccaagacttctaaaactgcagtcttttatcttttttttttcttttgccccttactaatcctcccttctgagttttgtatctatcaaagcactacttaaatgtttgggtacattagagatgcttgtacaatttactatagggtaggtgctgcaacttgtacctcagtatactttctgttcatgattgatttcaaggataagctgagatgcaacagtttaagatgtctcttctttgcctgtgtcccttgctcaattggggcataacatgagctgctgggaaaagatgctttgggagatgtcaaggtgcctgcaagctggactaagcgccacaatttttaaaaaagatatgcttcagagatggtaaagccagatttgtagtaatttagagatgaggatttgatgcagtactagaaagaggacagagggacgagatagagggtataaattagcaaggggagaaagagggaaagttaacaggagagcgagagaaaggacagaatctattacagggagttatttacgaagtgtttgttcataaactgcattagagtttagtgacaacaaattagaaagtacctaaaatgacaaaatactaggagctttcaatgcatataaacaatgggtgcatattccattttcatgcacaaaatgtacttcgtgtgttagaaaggaggcataatttgaagatgccattggtagggacaggaaaaatctttaggggtcgtttggttgggaattagttatcccaggattaactatcccgggattagctatcctgggataacttatctcaccatgtatattgattaacttttcccatcactatgatataaatggtgggatgagttattcccaaacaacataatttttttcatttcgtcactatttccttatcccatcactattatttttaattcctcacaccaaacgagtccttagactttacctattatcaggttcttacgcataatgcacccctctcaaataagaaagcaccatattctggatactactaaaagttgaggaagacatgttatactgaattgtaatagaaaatgaggaagatctttatatcatgctcttttccacatgggagagatggactttttttctgagggcagcaacgatgatgaggtttgtgcccaagaataggctatacatgtgtatcttgattggatgttttcagtccgttgactcgcccttcctgagaatgaatggagtaaattactatgaacacatattttgatgaatgccactaacaccaaattgcaggacccaattcttagataagcaaagtgtagcaggctaaggtgagcgttagccccttaaaatatttgtgacctagtatttttcagcttaaggtaattttcttggtatttacctccttcctactttgtgaagagcttgatatttagcctgtatggcacttatgtgagtattttggtggaacagaattggagagtttttgaagggtggaatatgactttgcattgcttaggcttgccttttattattgattaacttttggcacacccacgaggtccctgttagtatagatgaattattatcttatcatagagaagtgttattttatgggagttgtctactttttggtacacatattgtctaacatgttttctcctcataacatcagtaagctgttaattttttattttttttcgaaagctttgtgtcggaggttcatcagttatggagcttgttaaaccaaacaaaaaatgtcggtgattctttttgatacatatttgataacggtactgactactgacaatgatgagtaacaccaagaaggtgcaagagcaaacttacaaaaatgacaagtgttgcattcacatccattctcaagataattgaacttcatctccgctttacctacaaatataattaaattcccagctaattacggtgccgccacataataggaaagataaagcagtatggcatactttgtgcatttttttcatgttagtagatttattacaaatctagcacttttgtataaggtccttatagggaatgttatccttagtttatcctggctagtataggatatagacatatgacttgcatatgtatgtccttttattggctttctctctagttttggtgaattcttgattctattagtgagaacttcattttggctaatatgatgcttgtttttatgaggttgagattgattaggttggtcaatccaattaacatcaacttaaaacagtcctgagtagtgtaactttgctcttctgttttccctttaaaatttccaatcctgcaaggttgactcatctggtttaggatgtgaccttgccaaaagttctggtggtctagatttcttaacagaactcttttggctttaaacatcttgaaatatggatagattgtgttccttccatttttatgttcatatattgctggatttcttccattgatgttgttacaacaccgtacatttcaactaacagttttgactttttggtttttcctttattttaccttttttgagtgataatcctaaatttgagaaacacttgcacgtgcagagttcttgctgctgctgctaaaagagcaggagatgaggatgaaggtaagcttgcttggtttacatacttccaccttattgtctcaaccttttggtttctttgcagtcaaattgttttgttttgctatcttttctatttttaggttctcactaatcattttattttgtttccttttctatttttaggttctgatgctacagctgagaatggagtggataataaggacctcttaatgccagatctatgtgttatatgtctggagcaggaatacaactcagtttttgtcccgtaagaccctttcatgaaggcttttagttcactacatgttcaaatgttcagatttttgggacattgctatgctgcttaattgataaaattttaggaattgtatgcgaatcccaacttgtttggtgttggcacaatagaagtagagttcatataagtaataccaactaactcaagataaaactactgttttaagattaattaattcagatataactagtttacaacaaagatcttgaatggcaaattgtggaaaggagtgtgtaaatagagtggaatggatatggagactcgtgcagcttctcaaactattttggattgagggatatctgagttcatttgatttgattaatgacggatcagcttcctttttgtccctgtttaggttgctgaatgagattctgaaaaaatgagcatctctgagacttggcatcagatattcctatttttgtttttgtgttgggttgatccaagggtctttccgaaacagcctctctatcttcacaaggtagacctgactggtggtgttagttgttgtttaacattgtagtattgcccataagagcgggctggcaacactgttacttggaacagcagataggatactgaagctgtagtgtcctcaccctctaatataattaatttgacagcccaggcgctaatgcgaaacttttggacttttatgtttgtttggtttttgtgtagaaattaaaattttattttgttttcgcaggtgtggtcatatgtgctgttgcatgacgtgctcttcacacttgacaaactgcccactatgtaggagacggatcgagcaggttgtgaaaacttttcgccattga

SEQ ID NO: 11 - DNA, 1560 bp, Solanum lycopersicum
actcccaaattattttatggctacaaaaaaaaaaaaaaaaaaaaacgaagaagaagaagaagaagaagtggatggatggattatagagttgttgaaggagaggtagataaagagcatgaacataactgcactctcaagtttcctctcaaccactcttagcatcttatggagtgtcagcttcttcaaattccttttattttttaatcccaatttgtctcttcagttttactgttataaagccacatatgctctgcccttctgtaattgctctatttttgtttttccatgattgcttgtcgcctgaaacttgtgttgcttagtcataatttcattcgttaggtttaggttctattatcccttcgagctaccggaaatggttccatgggccggactctcttgctgtttgagtgcagctgctctttaccttctcggtaggagcagtggaagagatgctgaagttcttaaatccgttacaagggttaatcaattgaaggatcttgcacaactactagatactgcatccaaggtgttgcctctggtggttactatatctggaagggttggatcagatacaccaattaactgtgagtacagtggtctacgaggcgtgattgtggaagaaactgccgaacaacattttctgaaacacaatgatgcaggttcttggatacaggattctgccttgatgctctcaatgtgtaaagaagttccgtggtacctggatgatggcacaggtcgcacttttattgttggtggccgtggtgccacgggtttggtactgacagttggaagtgaagccttcgaggaagcagggagatcatttgtgcgagggacattggattatcttcaaggtcttaagatgcttggagtaaagaggattgaacgtgtgctgccagttggtactcctttgactgttgttggcgaggctgtcaaagatgacattgggacagttcggatccagcgaccacacaaaggcccattttatatctctcataaaactattgaccagctcattgcaaatcttgggagatgggcaaggtggtacaagtatgcttcaatgggttttactgctgtcggtgtatatctacttgtgaagcatacttttcattatatgatggaaagaaagcgtcactgggaattgcgcaggagagttcttgctgctgctgctaaaagagcaggagatgaggatgaaggttctgatgctacagctgagaatggagtggataataaggacctcttaatgccagatctatgtgttatatgtctggagcaggaatacaactcagtttttgtcccgtgtggtcatatgtgctgttgcatgacgtgctcttcacacttgacaaactgcccactatgtaggagacggatcgagcaggttgtgaaaacttttcgccattgagcagaaagctgccttgttgaaatggccattttctgactcgctcataaaaatcattcggatattaacttgtagattgtattgaaagctggattttgtcagagttttatgtcattatgatgtatgtataatagcctctctccatctcaacggttaaactagca

SEQ ID NO: 12 - DNA, 32 bp, Artificial Sequence
  Primer SP1-CDS-F
aaaaagcggcttcatgattccttggggtggag

SEQ ID NO: 13 - DNA, 35 bp, Artificial Sequence
  Primer SP1-CDS-R
agaaagctgggtttcagtgacgatatgtcttaacc

SEQ ID NO: 14 - DNA, 32 bp, Artificial Sequence
  Primer SP1-nonstop-R
agaaagctgggttgtgacgatatgtcttaacc

SEQ ID NO: 15 - DNA, 45 bp, Artificial Sequence
  Primer SP1-C330A-R
gacgatatgtcttaaccgccagatctattcgtctccgagcaagtg

SEQ ID NO: 16 - DNA, 36 bp, Artificial Sequence
  Primer SPA-TM1-F
aaaaagcaggctccatgcggagtagtggcagggatg

SEQ ID NO: 17 - DNA, 38 bp, Artificial Sequence
  Primer SP1-TM2-R
tagaacagagtcaattttgtacaaccttgaccattttc

SEQ ID NO: 18 - DNA, 35 bp, Artificial Sequence
  Primer SP1-TM2-F
tcaaggttgtacaaaattgactctgttctagagag

SEQ ID NO: 19 - DNA, 39 bp, Artificial Sequence
  Primer SP1-TM2-Flex1
ctccaccgctaccgccgcctcctttgtacaaccttgacc

SEQ ID NO: 20 - DNA, 41 bp, Artificial Sequence
  Primer SP1-TM2-Flex 2
gcggtagcggtggaggtggcagcattgactctgttctagag

SEQ ID NO: 21 - DNA, 24 bp, Artificial Sequence
  Primer SP1-pGEX-IMS-F
ggatcccggagtagtggcagggat

SEQ ID NO: 22 - DNA, 29 bp, Artificial Sequence
  Primer SP1-pGEX-IMS-R
aagctttttgtacaaccttgaccattttc

SEQ ID NO: 23 - DNA, 34 bp, Artificial Sequence
  Primer SP1-cyt-F
aaaaagcaggctccattgactctgttctagagag

SEQ ID NO: 24 - DNA, 34 bp, Artificial Sequence
  Primer SPL1-CDS-F
aaaaagcaggctccatgatacatttggctggatt

SEQ ID NO: 25 - DNA, 37 bp, Artificial Sequence
  Primer SPL1-CDS-R
agaaagctgggtttcaatggcggtaaattttcaaaac

SEQ ID NO: 26 - DNA, 34 bp, Artificial Sequence
  Primer SPL1-nonstop-R
agaaagctgggttatggcggtaaattttcaaaac

SEQ ID NO: 27 - DNA, 33 bp, Artificial Sequence
  Primer SPL2-CDS-F
aaaaagcaggctccatgtcctcgccggagcgtg

SEQ ID NO: 28 - DNA, 33 bp, Artificial Sequence
  Primer SPL2-nonstop-R
agaaagctgggttagagtaatatacacgcatag

SEQ ID NO: 29 - DNA, 32 bp, Artificial Sequence
  Primer YFP-F
aaaaagcaggctccatggtgagcaagggcgag

SEQ ID NO: 30 - DNA, 32 bp, Artificial Sequence
  Primer YFP-nonstop-R
agaaagctgggttcttgtacagctcgtccatg

SEQ ID NO: 31 - protein, 341 aa, Solanum lycopersicum
MVPWAGLSCCLSAAALYLLGRSSGRDAEVLKSVTRVNQLKDLAQLLDTASKVLPLVVTISGRVGSDTPINCEYSGLRGVIVEETAEQHFLKHNDAGSWIQDSALMLSMCKEVPWYLDDGTGRTFIVGGRGATGLVLTVGSEAFEEAGRSFVRGTLDYLQGLKMLGVKRIERVLPVGTPLTVVGEAVKDDIGTVRIQRPHKGPFYISHKTIDQLIANLGRWARWYKYASMGFTAVGVYLLVKHTFHYMMERKRHWELRRRVLAAAAKRAGDEDEGSDATAENGVDNKDLLMPDLCVICLEQEYNSVFVPCGHMCCCMTCSSHLTNCPLCRRRIEQVVKTFRH

SEQ ID NO: 32 - protein, 343 aa, Brassica rapa
MIHWGGVTCCLSAAALYLLGSSSGRDAEVLKTVTRVNQLKELAQLLELDSSKLLPFIVAVSGRVGSDTPIKCEHSGIRGVIVEETAEQHFLKHNETGSWVQDSALMLSMSKEVPWFLDDGTSRVNVVGARGATGFALTVGSEVFEESGRSLVRGTLDYLQGLKMLGVKRIERVLPTGMPLTIVGEAVKDDIGDLRIQKPERGPFYVSPKSLDQLISNLGKWSRWYKYASMGLTVFGVFLITKHVIDHVLERRRHRELQKRVLDAAAKRAEETEGSNGAHESVSDSTKKEGAVPDLCVICLEHNYNAVFVPCGHMCCCTACSSHLTSCPLCRRRIDQVVKTYRH

SEQ ID NO: 33 - protein, 339 aa, Carica papaya
MIPWGGITCCMSAAALYLLGRSSGRDADILRKVTRVNQLKELAQLLDIESKIMPLIVAISGRVGSETPISCEYSGLRGVIVEETAEQHFLKHNDAGSWIQDSALMLSMSKEVPWYLDDGTARVFVVGARGATGFVLTVGSEVFEESGRSLVRGTLDYLQGLKMLGVKRIERVLPTGTSLTVVGEMTLEQFRYEDRTNGLFTCLQINGSPDFQLGKWARWYKYASMGLTFLGVFLIAKRSIEYVLERRRRRELQKRVLAAAAKRSGQDNEGSNIIPENGLDGAKREHLMPDLCVICLEQEYNAAFVPCGHMCCCMTCSSHLTNCPLCRRRIEQVLRTFRH

SEQ ID NO: 34 - protein, 344 aa, Camellia sinensis
MIPWGGISCCLSGAALYLLGRSSGRDAELLKTVTRVNQLKELAHLLDSGSKVLPFIVTVCGRVGSETPISCEYSGLRGVIVEETAEQHFLKHNDAGSWIQDSALMLSMSKEVPWYLDDGTGRAFVVGARGATGFVLTVGSEVFEESGRSLVRGTLDYLQGLKMLGVKRIERLLPTGTSLTVVGEAVKDDIGTVRIQRPHKGPFYVSPKTIDELIENLGKWARWYKYASFGLTIFGTFLIAKRAIHYILQRKRRWELHRRVLAAAAVKRSEQDNEGTNGQAENGSDGTQRDRVMPDLCVICLEQEYNAVFVPCGHMCCCIICSWHLTNCPLCRRRIDQVVRTFRH

SEQ ID NO: 35 - protein, 344 aa, Citrus clementina
MIPWGGISCCLSGAALYLLGRSSGRDAELLKTVTRVNQLKELAHLLDSGSKVLPFIVTVCGRVGSETPISCEYSGLRGVIVEETAEQHFLKHNDAGSWIQDSALMLSMSKEVPWYLDDGTGRAFVVGARGATGFVLTVGSEVFEESGRSLVRGTLDYLQGLKMLGVKRIERLLPTGTSLTVVGEAVKDDIGTVRIQRPHKGPFYVSPKTIDELIENLGKWARWYKYASFGLTIFGTFLIAKRAIHYILQRKRRWELHRRVLAAAAVKRSEQDNEGTNGQAENGSDGTQRDRVMPDLCVICLEQEYNAVFVPCGHMCCCIICSSHLTNCPLCRRRIDQVVRTFRH

SEQ ID NO: 36 - protein, 342 aa, Cucumis sativus
MLPWGGISCCLSAAALYLLGRSSGRDAELLKSVTRVNQLKELAQLLEAEHLLPLVVAISGRVSSDTPINCEFSGLRGVIVEETAEQHFLKHNDAGSWIQDSALMLSMSKEVPWYLDDGTGRAFVLGARNATNFILPVVSEVFEESGRSLMRGTLDYLQGLKMLGVKRIERVLPTGTSLTVVGEAAKDDIGTIRIQRPHKGPFYVSPKTIDQLISNLGKWARWYKYASMGLSIFGLYLVTKHVILYLMERRRRWELQKRVLAAAAKRSSQENEGEIEKASNGTDGTKRDRSMPDLCVICLERDYNAVFVPCGHMCCCVACCSHLTNCPLCRRRIELVVKTFRH

SEQ ID NO: 37 - protein, 340 aa, Prunus persica
MLPWGGLSCCLSAAALYLLGRSSGRDADILKSATRINQLKELAKLLDSECILPLVVAISGRVSSETPITCEFTGLRGVVVEETAEQHFLKHNDAGSWIQDSALMLSMSKEVPWYLDDGTGRVHVVGARGATGFVLPVASEVFEESGRSLVRGTLDYLQGLKMLGVKRIERVLPTGTSLSVVGEAVKDDIGTIRIQRPHKGPFYVSPKTIDQLIANLGKWARWYKYASLGLTVFGVYLVAKHSVQYILERRRRWELQRRVLAAAAKRSGEDNEGSNEKDDNVLDGSKRLMPDLCVICLEHEYNAVFVPCGHMCCCTTCSLHLTNCPLCRRRIDQAVKTFRH

SEQ ID NO: 38 - protein, 342 aa, Fragaria vesca
MLPWGGLSCCLSAAALYLLGRSSGRDADILKTATRVSQLKELAKLLDSESILPLVVAISGRVSSETPITCEFSGLRGVVVEETAEQHFLKHNDAGSWIQDSALMLSMSKEVPWYLDDGTGRVHVVGARGATGFTLPVASEVFEESGRSLVRGTLDYLQGLKMLGVKRIERVLPTGTSLSVVGEAVKDDIGTIRIQRPHKGPFYISPKTIDQLIANLGKWARLYKYASLGLGVFGVYLIAKHSVHYIMERRRRWELQKRVLAAAAKRSGEDIEGSNDIEYNASEAPKKDRLMPDLCVICLEQEYNAVFVPCGHMCCCTTCSLHLTNCPLCRRRIEQVVKTFRH

SEQ ID NO: 39 - protein, 339 aa, Glycine max
MIPWGGLSCCLSAAALYLLGRSSGRDAEILKSVTRVNQLKELAQLLDAEILPLIVTISGRVSSETPINCEFSGLRGVIVEETAEQHFLKHNDAGSWIQDSALMLSMSKEVPWYLDDGTDRVHVVGARGAAGFALPVGSEAFEESGRSLVRGTLDYLQGLKMLGVKRIERVLPVGTSLTVVGEAAKDDVGAFRIQRPHKGPFYVSPKTIDQLIANLGKWARWYKYASMGLTVFGAYLIAKHAIRYILERRRRSELQRRVLAAAAKKSGQNNDVEKADGLSDGVKKDRLMPDLCVICLEQEYNAVFVPCGHMCCCTTCSSHLTNCPLCRRQIEKVVKTFRH

SEQ ID NO: 40 - protein, 339 aa, Glycine max
MIPWGGLSCCLSAAALYLLGRSSGRDAEILKSVTRVNQLKELAQLLDAEILPLIVTISGRVSSETPINCEFSGLRGVIVEETAEQHFLKHNDAGSWIQDSALMLSMSKEVPWYLDDGTDRVHVVGARGASGFALPVGIEAFEESGRSLVRGTLDYLQGLKMLGVKRIERVLPVGTSLTVVGEAAKDDVGAIRIQRPHKGPFYVSPKTIDQLIANLGKWARWYKYASVGLTVFGAYLIAKHAIRYILERRRRSELQRRVLAAAAKKSGQNNDVEKADSLSDGAKKDRLMPDLCVICLEQEYNAVFVPCGHMCCCTACSSHLTNCPLCRRQIEKVVKTFRH

SEQ ID NO: 41 - protein, 342 aa, Proteus vulgaris
MKMIPWGGLSCCLSAAALYLLGRSSGRDAEILKSVTRVNQLKELAQLLDAEILPLIVTISGRVGSETPISCDFSGLRGVIVEETAEQHFLKHNDAGSWIQDSALMLSMSKEVPWYLDDGSDRVHVVGARGATGFALPVGSEAFEESGRSLVRGTLDYLQGLKMLGVKRIERVLPVGTSLTVVGEAAKDDVGTIRIQRPHKGPFYVSPKTIDQLIANLGKWARWYKYASMGLTVFGAYLIAKHAIRFILERRRRSELQRRVLAAAAAKKSGPNNDVEKADSLSDVAKRDHLMPDLCVICLEQEYNAVFVPCGHMCCCTACSSHLTNCPLCRRQIEKVVKTFRH

SEQ ID NO: 42 - protein, 344 aa, Medicago truncatula
MAMVPWRGVGCCLSAAALYLLGRTSGYVDILKSVNRVNQLRELAQLLDEEIFPLVVAISGRVGSETPISCEFSGLRGVIIEETAEQHFLKHSDAGSWIQDSALMQSRSNEVPWYLDDGTGRVRVVGAQGATGFVLPVGSEAFEESGRLPVRGTSDYVQGLKVGVLMLGVKRIERVLPVGTSLTVVGEAAKDDVGTIRIQRPSKGPFYVSPKTIDELIANIGRWARWYKYASAGLTVLSVYMIANHAVRYILERRRRNELEKRVLAAAAKISGQDNGGEMDDSLSDGAKRERAMPNLCVICLEQEYNSVFVPCGHMCCCTACSSHLTSCPLCRRQIEKAVKTFRH

SEQ ID NO: 43 - protein, 343 aa, Populus balsamifera subsp. trichocarpa
MMIPWGGISCCLSGAALYLLGRSSGRDAEVLKSVAKVNQLKELAKLLDIESKVLPLVVAISGRVGAESPISCEFSGLRGVIVEETAEQHFLKHNDAGSWIQDSALMLSMSKEVPWYLDDGTDRVYVVGARGASGFVLTVGSEVFEESGRSLVRGTLDYLQGLKMLGVKRIERVLPTGTSLTVVGEAVKDDIGTVRIQRPHKGPFYVSPKSIDELIGNLGKWARWYKYASLGLTVFGAFLITKHVIRYIMERRRRWELQSRVLAAAKRSGQDNEGSNDKAENGSDGAKRERPIPDLCVICLEQEYNAVFLPCGHMCCCITCCSQLSNCPLCRRRIEQVVKTFRH

SEQ ID NO: 44 - protein, 343 aa, Vitis vinifera
MIPWGGISCCLSAAALYLLGRSSGRDAEALKSVTRVQQLKDLVQLLDTACKVLPLVVTVSGRVGSDTPIKCEYSGLRGVIVEETAEQHFLKHNDAGSWIQDSALMLSMSKEVPWYLDDDTGRAYIVGARGATGLVLTVGSEVFEESGRSLVRGTLDYLQGLKMLGVKRIERVLPTGTPLTVVGEAIKDDVGTIRIQRPHKGPFYVSPKSIDHLVANLGKWARWYRYASLGFTVFGVYLIAKSAIQYVMERKRCWELRKRVLAAASKKSGQDSEDPDEKDENGSDNTKRDRLMPDLCVICLEQEYNAVFVPCGHMCCCTMCSSQLTNCPLCRRRIEQVVRTFRH

SEQ ID NO: 45 - protein, 341 aa, Solanum lycopersicum; identical to SEQ ID NO: 31

SEQ ID NO: 46 - protein, 342 aa, Solanum tuberosum
MVPWGGLSCCLSAAALYLLGRSSGRDAEVLKSVTRVNQLKDLAQLLDTASKVLPLVVTISGRVGSDTPINCEYSGLRGVIVEETAEQHFLKHNDAGSWIQDSALMLSMCKEVPWYLDDDTGRTFVVGGRGATGLVLTVGSEIFEEAGRSFVRGTLDYLQGLKMLGVKRIERVLPVGTPLTVVGEAVKDDIGTVRIQRPHKGPFYISHKTIDQLIANLGRWARWYKYASMGFTAVGVYLLVKHTFHYMMERKRHWELRRRVLAAAAKRAGHEDEGSNATAENGVDNKKDLLMPNLCVICLEQEYNSVFVPCGHMCCCMTCSSHLTNCPLCRRRIEQVVKTFRH

SEQ ID NO: 47 - protein, 343 aa, Sorghum bicolor
MMIPWGGVGCCLSAAALYLLGRSSGRDAEVLRSVARAGSMKDLAAILDTASKVLPLVVAVSGRVSSDTPLICQQSGMRGVIVEETAEQHFLKHNDAGSWIQDSAVMLSVSKEVPWYLDDGTGRVYVVGARSAAGLILTVASEVFEESGRTLVRGTLDYLQGLKMLGVKRTERVLPTGTSLTVVGEAIRDDVGTIRIQRPHKGPFYVSPKSIDQLIMNLGKWAKLYRLASMGFATFGVFLLAKRAIQHFLERKRRHELQKRVLNAAAQRQAREAEGSNGSSDTEPNSKKDQLVLDICVICLEQEYNAVFVPCGHMCCCMACSSHLTNCPLCRRRIDQAVRTFRH

SEQ ID NO: 48 - protein, 343 aa, Zea mays
MMIPWGGVGCCLSAAALYLLGRSSGRDAEVLRSVARAGSMKDLAAILDTASKVLPLVVAVSGRVGSDTPLICQQSGMRGVIVEETAEQHFLKHNDAGSWIQDSAVMLSVSKEVPWYLDDGTGRVYMVGARSAAGLILTVASEVFEESGRTLVRGTLDYLQGLKMLGVKRTERVLPTGISLTVVGEAIKDDVGTIRIQRPHKGPFYVSPKSIDQLIMNLGKWAKLYRLASMGFATFGAFLLAKRAIQHFLERKRRHELQKRVLNAAAQRQAREAEGSIGSSDTEPNSKKDQLVLDICVICLEQEYNAVFVPCGHMCCCMACSSHLTNCPLCRRRIDQAVRTFRH

SEQ ID NO: 49 - protein, 343 aa, Zea mays
MMIPWGGVGCCLSAAALYLLGRSSGSDAEVLRSVARAGSMKDLAAILDTASKVLPLVVAISGRVGSDTPLICQQSGMRGVIVEETAEQHFLKHNDAGSWIQDSAVMLSVSKEVPWYLDDGTGRVYVVGARSAAGLILTVASEVFEESGRTLVRGTLDYLQGLKMLGVKRTERVLPTGTSLTVVGEAIKDDVGTIRIQRPHKGPFYASSKSIDQLIVNLGKWAKLYRIASMGFATFGVFLLAKRALQHFLERRRRHELQKRVLNAAAQRQAREAEGSKGTSDAEPNSKKDQLVLDICVICLEQEYNAVFVPCGHMCCCVACSSHLTNCPLCRRRIDQAVRTFRH

SEQ ID NO: 50 - protein, 343 aa, Setaria italica
MMIPWGGVGCCLSAAALYLLGRSSGRDAEVLRSVARAGSMKDLAAILDTASKVLPLVVAVSGRVGSDTPLICQQSGMRGVIVEETAEQHFLKHNDAGSWIQDSAVMLSVSKEVPWYLDDGTGRVYVVGARAAAGLILTVASEVFEESGRTLVRGTLDYLQGLKMLGVKRTERVLPTGTSLTVVGEAIKDDVGTIRIQRPHKGPFYVSPKSIDQLILNLGKWAKLYRLASMGFATFSVFLLAKRAIQHFLERKRRHELQKRVLNAAAQRQAREAEGGKGTSNTEPNSKKDQLVLDICVICLEQEYNAVFVPCGHMCCCMACSSHLTNCPLCRRRIDQAVRTFRH

SEQ ID NO: 51 - protein, 343 aa, Panicum virgatum
MMIPWGGVGCCLSAAALYLLGRSSGRDAEVLRSVARAGSMKDLAAILDTASKVLPLVVAVSGRVGSDTPLICQQSGMRGVIVEETAEQHFLKHNDAGSWIQDSAVMLSVSKEVPWYLDDGTGRVYVVGARAAAGLILTVASEVFEESGRTLVRGTLDYLQGLKMLGVKRTERVLPTGTSLTVVGEAIKDDVGTIRIQRPHKGPFYVSPKSIDQLILNLGKWAKLYRLASMGFATFGVFLLAKRAIQHFLERKRRHELQKRVFNAAAQRQARESEGGNGTSDTEPNSKKDQLVLDICVICLEQEYNAVFVPCGHMCCCMACSSHLTNCPLCRRRIDQAVRTFRH

SEQ ID NO: 52 - protein, 343 aa, Oryza sativa
MLIPWGGVGCCLSAAALYLLGRSSGRDAEVLRSVARAGSTKDLAAILDTASKVLPLVVAVSGRVGSDTPLICQQSGMRGVIVEETAEQHFLKHNDAGSWIQDSAVMLSVSKEVPWYLDDGTGRVFVVGARGAAGLVLTVASEVFEESGRTLVRGTLDYLQGLKMLGVKRTERVLPTGTSLTVVGEAIKDDVGTIRIQRPHKGPFYVSPKSIDQLIMNLGKWAKLYQLASMGFAAFGVFLLAKRALQHFLERKRRHELQKRVHAAAAQRQAREAEGGNGTSDVDSNNKKDQLVLDICVICLEQEYNAVFVPCGHMCCCMNCSSHLTNCPLCRRRIDQAVRTFRH

SEQ ID NO: 53 - protein, 343 aa, Brachypodium distachyon
MMVPWGGVGCCLSAAALYLLGRSSGRDAEVLRSVTRTGSLKDLAAILDTASKVLPLVVAVSGRVSSDTPLICQQSGMRGVIVEEMAEQHFLKHNDAGSWIQDSAVMLSVSKEVPWYLDDGTGRVYVVGARAAAGLVLTIASEVFEESGRTLVRGTLDYLQGLKMLGVKRTERVLPTGTSLTVVGEAIKDDVGTIRIQRPHKGPFYASPKSIDQLILNLGKWAKLYQLASMGFAAFGVFLLAKRALQHFLQKKRQHELNKRVRAAAAQRQAREAEGADGTSNGDPNSKKDQLVLEICVICLEQEYNAVFVPCGHMCCCMNCSSHVTNCPLCRRRIDQAVRTFRH

SEQ ID NO: 54 - protein, 338 aa, Arabidopsis thaliana
MIHLAGFTCCLGGVALYLLTRSTGRDIKSITRVYQLKDLEQLVEVESKVVPLIIAVSGDVGSETPIKCEHSYVLGVFLKRTAEQQVLRRNWRFSWVRNSTLMQPMTKEVPWYLDDGTGRVNVDVSQGELGLALTVGSDVFEKAEPVSLVQGALGYLKGFKILGVRHVERVVPIGTPLTVVGEAVRDGMGNVRIQKPEQGPFYVTYIPLDQLISKLGDLSRRFKYASMGLTVLGVILISKPVIEYILKRIEDTLERRRRQFALKRVVDAAARRAKPVTGGGTSRDGDTPDLCVVCLDQKYNTAFVECGHMCCCTPCSLQLRTCPLCRERIQQVLKIYRH

SEQ ID NO: 55 - protein, 348 aa, Brassica rapa
MEPWSGLCCIGAVALYLLSRRTARDVDFLKSVTRVDQLKDLESAVLPSIVAVSGTVGSETPIKCEHSGILSVILQETAEQQFLKRNWKFSWVQDTALMLPVCKEVPWFLDDGTGRVIVEGARSGIGFELTVGGEVFEKPEASSLVRGTLDFLRGLEMLGIRRIERVLPVGTRLTVVGQTIKDGVGDVRIQKPDQGPFYVSPIPLDHLISKVGKWSRRFKKASMGLAVVGVILISKPVIKYILVRTGDFLERRQQRLLKKRVVDAAAKRKKLAAPKREERVTSKGLENGKSRDGDEHDRCVVCLERKCDAAFVPCGHMCCCLTCALKLLGKPCPLCRKRGIRILKIYRN

SEQ ID NO: 56 - protein, 371 aa, Zea mays
MSARDRETAEALVRLAASLDGAVLGLGTAAVAVASWVKYLAVSGQLRLVASAATASIADARSLLSGDGAEPRIAAVRGYVRAHDKFFRAPFSGEAGVVTKHTQMCLFTEWRGIFGWTFDLHALLFRSWKEQIVTSFRSVPFVLVSTELGNPTGVVHINVDKADQPLPLTTVFHKLIPLETTPYTLFQTIIGNGYPIALLDEEKILPIGKKITAIGLCQAKDAESVEITSCPEIPFFLSELTKDEMQAQLASRARILFWGSIVLGTLSVCLVGHAIYRGWTRIKLRREARHARQMFEEAEDAIHRDDSSDDDEIGDGQLCVVCLRKRRRAAFIPCGHLVCCSECALTIERTPHPLCPMCRQDIRYMMRVYDS

SEQ ID NO: 57 - DNA, 21 bp, Artificial sequence
  amRNA
tcatatgaccacacgcgacaa

SEQ ID NO: 58 - DNA, 21 bp, Artificial sequence
  amRNA
taagtagatatacactgacag

(19) United States
(12) Patent Application Publication
(10) Pub. No.: US 2016/0108416 A1
(43) Pub. Date: Apr. 21, 2016

(54) ATSP1, AN E3 UBIQUITIN LIGASE, AND ITS USE

(71) Applicant: UNIVERSITY OF LEICESTER, Leicester (GB)

(72) Inventor: PAUL JARVIS, LEICESTER (GB)

(21) Appl. No.: 14/643,507

(22) Filed: Mar. 10, 2015

Related U.S. Application Data
(63) Continuation-in-part of application No. PCT/GB2013/052338, filed on Sep. 6, 2013.

(30) Foreign Application Priority Data
Sep. 10, 2012 (GB) 1216090.9
Oct. 19, 2012 (GB) 1218837.1

(51) Int. Cl. C12N 15/82; C12N 9/00

(52) U.S. Cl. CPC C12N 15/8245; C12N 15/8266; C12N 15/8261; C12N 15/8267; C12N 15/8249; C12N 15/8271; C12N 9/93; C12N 15/8273; C12Y 603/02019

(57) ABSTRACT

The invention relates to plants with improved phenotypes and related methods. These improved phenotypes are conferred by altering the expression of the SP1 gene which is involved in plastid development or altering the activity of the SP1 protein.

### RELATED APPLICATIONS AND INCORPORATION BY REFERENCE

This application is a continuation-in-part application of international patent application Serial No. PCT/GB2013/052338 filed 6 Sep. 2013, which published as PCT Publication No. WO 2014/037735 on 13 Mar. 2014, which claims benefit of GB patent application Serial No. 1216090.9 filed 10 Sep. 2012 and GB patent application Serial No. 1218837.1 filed 19 Oct. 2012.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### SEQUENCE LISTING

The instant application contains a Sequence Listing which has been submitted electronically in ASCII format and is hereby incorporated by reference in its entirety. Said ASCII copy, created on Jan. 4, 2016, is named 48036_00_2001_SL.txt and is 114,113 bytes in size.

### FIELD OF THE INVENTION

The invention relates to transgenic plants with improved yield-related traits, including delayed leaf senescence, improved seedling survival, fruit ripening, grain size/starch content and/or stress tolerance. Also within the scope of the invention are related methods, uses, isolated nucleic acids and vector constructs.

### INTRODUCTION

The ever-increasing world population and the dwindling supply of arable land available for agriculture fuels research towards increasing the efficiency of agriculture and providing food security. Conventional means for crop and horticultural improvements utilise selective breeding techniques to identify plants having desirable characteristics. However, such selective breeding techniques have several drawbacks, namely that these techniques are typically labour intensive and result in plants that often contain heterogeneous genetic components that may not always result in the desirable trait being passed on from parent plants. Advances in molecular biology have allowed mankind to modify the germplasm of animals and plants. Genetic engineering of plants entails the isolation and manipulation of genetic material (typically in the form of DNA or RNA) and the subsequent introduction of that genetic material into a plant. Such technology has the capacity to deliver crops or plants having various improved economic, agronomic or horticultural traits, including increased yield under stress conditions.

A trait of particular economic interest is increased yield, including under stress conditions. Even in “normal” crop growing conditions crop plants are commonly under mild to moderate stresses such as, without limitation, from sub- or supra-optimal temperature, light stress or moisture deficit. Yield is normally defined as the measurable produce of economic value from a crop. This may be defined in terms of quantity and/or quality. Yield is directly dependent on several factors, for example, the number and size of the organs, plant architecture (for example, the number of branches), seed production and leaf senescence. Root development, nutrient uptake, stress tolerance and early vigour may also be important factors in determining ultimate yield. Optimizing the abovementioned factors may therefore contribute to increasing crop yield.

Plastids are plant organelles and include chloroplasts, amyloplasts and chromoplasts (1). Chloroplasts are so named because they contain chlorophyll. Chromoplasts lack chlorophyll but contain carotenoids; they are involved in fruit ripening. Leucoplasts are non-pigmented plastids, which store a variety of energy sources in non-photosynthetic tissues. Amyloplasts and elaioplasts are examples of leucoplasts that store starch and lipids, respectively. Plants can adapt to environmental and developmental cues by altering the phenotype of plastid organelles. For example, in dark-grown plants exposed to light, etioplasts differentiate into chloroplasts (a process known as de-etiolation). A unique feature of the plastid family is the ability of its members to interconvert in response to developmental and environmental cues, for example during de-etiolation or fruit ripening (1). Such plastid interconversions are linked to reorganization of the organellar proteome (2, 3).

Chloroplasts are the site of photosynthesis in plants, algae and some protists, and so mediate much of the world's primary productivity. As photosynthesis is the only significant mechanism of energy-input into the biosphere, chloroplasts are essential for plants and animals alike; thus, agriculture is wholly dependent on chloroplast biogenesis. Chloroplasts or other plastids also mediate many biosynthetic processes (starch, amino acids, fatty acids). Many of these products are vital in mammalian diets, and knowledge on plastid biogenesis may enable improvements in their quantity or quality. Since plastids are so integral to cellular metabolism, plastid biogenesis defects can cause plants to die during (pre)embryonic development.

Chloroplasts contain ˜3000 proteins but only ˜100 are encoded by the plastid's own genetic material (the “plastome”). Over 90% of the thousands of proteins in plastids are nucleus-encoded and imported from the cytosol post-translationally (1). The translocon at the outer envelope of chloroplasts (TOC) recognizes chloroplast pre-proteins and initiates their translocation (4-6). The TOC machinery comprises the Omp85-related channel Toc75, and the receptor GTPases Toc34 and Toc159. The receptors protrude into the cytosol where they contact incident pre-proteins, and exist in multiple isoforms of differing specificity: in Arabidopsis, the major isoforms (atToc33 and atToc159) recognize abundant precursors of the photosynthetic apparatus, whereas the minor isoforms (atToc34 and atToc132/atToc120) recognize housekeeping pre-proteins (7-10). Receptor isoform levels vary developmentally depending on biochemical requirements of the plastids.

Plastids offer many opportunities for agricultural exploitation, for example in delaying leaf senescence (‘stay-green’ trait) to allow longer “source” productivity and better crop health, altering seedling development for better crop establishment or manipulating fruit ripening.

For example, genotypes have been identified in a number of crop species, including cereals, whose leaves remain green for longer than those of the parental genotypes: these are defined as ‘stay green’. During plant senescence, leaf colour changes from green to yellow or red. Colour change of leaves during senescence is caused by chlorophyll degradation, combined with carotenoid retention or anthocyanin accumulation. Chloroplasts convert to gerontoplasts during leaf senescence. The ‘stay-green’ trait is responsible for the preservation of green colouration in the stem and leaf, during physiological maturity. Moreover, this trait has also been shown to play an important role in the increase in grain size by extending the period of photosynthetic production in the leaves (“source”) increasing translocation of carbohydrate assimilate to the seed/grain (“sink”). ‘Stay-green’ may also improve yield by reducing the development of pathogens (e.g. fungi) on crops particularly towards the end of the growing season.

The total photosynthesis over the life of annual crops can be increased by extending the duration of active photosynthesis. Furthermore, maintaining the supply of assimilated carbon to grain during the grain-filling period of determinate crops ensures that the mass per grain is maximized. Delaying leaf senescence is one way by which this can be achieved. Delaying leaf senescence may be particularly advantageous under certain environmental conditions, such as high temperature, that tend to accelerate senescence and thus decrease the supply of assimilates to the grain. Developing plants with delayed leaf senescence is therefore a desirable goal in agriculture.

Adverse climate conditions and human activity as well as biological agents are stress effectors for plants and seriously affect their productivity and survival as plants adapt to changing environmental conditions by modifying their growth. When subjected to environmental stress, plants actively reduce their vegetative growth to conserve and redistribute resources and thus increase their chance of survival if the stress becomes severe. However, when the stress does not threaten survival, growth inhibition is counterproductive because it leads to an unnecessary drop in productivity and substantial yield penalties. Losses in productivity due to stress sometimes reach more than 50%. Even moderate stress can have significant impact on plant growth and thus yield of agriculturally important crop plants. Therefore, finding a way to improve yield, in particular under stress conditions, is of great economic interest.

Citation or identification of any document in this application is not an admission that such document is available as prior art to the present invention.

### SUMMARY OF THE INVENTION

The invention is aimed at providing transgenic plants with improved yield that are beneficial to agriculture, including delayed leaf senescence, altered seedling development, fruit ripening and increasing stress tolerance and grain traits.

Surprisingly, the inventor has shown that modulating expression of a nucleic acid encoding a SP1 polypeptide gives plants having enhanced yield-related traits relative to control plants.

Development of chloroplasts and other plastids depends on the import of thousands of nucleus-encoded proteins from the cytosol. Import is initiated by TOC complexes in the plastid outer membrane which incorporate multiple, client-specific receptors. Modulation of import is thought to control the plastid's proteome, developmental fate, and functions. While the main TOC components were identified over a decade ago, regulatory mechanisms that govern their action are poorly understood. To shed light in this area, the inventor screened an ethyl methanesulfonate-mutagenized population for second-site suppressors of the atToc33 knockout mutation, plastid protein import1 (ppi1; which causes chlorosis due to defective protein import) (7), and identified suppressor of ppi1 locus1 (sp1). Double-mutant sp1 ppi1 plants were larger and greener than ppi1, and exhibit improved chloroplast ultrastructural organization and protein import capacity (FIG. 1). Recovery mediated by sp1 is specific, as two other mutations that cause chlorosis were not suppressed. The only other mutation found to be suppressed by sp1 was a hypomorphic allele of the gene encoding Toc75 (toc75-III-3) (11, 12), implying a close functional relationship between SP1 and the TOC apparatus.

The AtSP1 protein was identified as an E3 ligase in a publication that looked at a wide range of different RING proteins in Arabidopsis (13), but neither its localization nor its targets were determined. Another study erroneously identified AtSP1 and AtSPL1 as homologues of the Drosophila DIAP1 protein (31).

As shown herein, the SP1 protein associated with TOC complexes and mediated ubiquitination of TOC components, promoting their degradation. Mutant sp1 plants performed developmental transitions that involve plastid proteome changes inefficiently, indicating a requirement for reorganization of the TOC machinery, depending on the plant stage, tissue and related circumstances. The inventor has shown that SP1 plays a role in the reorganization of the TOC machinery and in the regulation of plastid biogenesis. This is important during developmental phases in which plastids convert from one form to another through organellar proteome changes. Plastid transitions are commercially important and either delaying or accelerating plastid development can produce a beneficial plant phenotype. An example is plastid transition during fruit ripening when chloroplasts differentiate into chromoplasts which accumulate carotenoid pigments of dietary significance, for example in tomatoes. A further example is amyloplast development in grain, roots and tubers. Another important transition is that of heterotrophic etioplasts to chloroplasts in seedlings. This is essential for initiation of photoautotrophic growth after seed germination beneath the soil and for seedling emergence, growth and survival. The inventor found that sp1 single mutants de-etiolated inefficiently, are less able to utilise and compete for light, hence displaying reduced growth and survival rates linked to delayed organellar differentiation (FIG. 4, A to E), reduced accumulation of photosynthetic proteins, and imbalances in TOC receptor levels. At the other end of the life-cycle, chloroplasts transform into gerontoplasts as catabolic enzymes accumulate to recover resources from the organelles of senescent leaves for use elsewhere in the plant. This response is characterised by declining photosynthetic performance, and can be induced prematurely by dark treatment. The sp1 mutation also attenuated this transition (FIGS. 4, F and G), while SP1 overexpression accelerated both senescence and de-etiolation (FIG. 4), presumably due to the hastening of organellar proteome changes. Delaying the transition of chloroplasts to gerontoplasts delays senescence of green tissue and produces a ‘stay-green’ effect.

Thus, a skilled person would appreciate that a plant SP1 gene or SP1 peptide can be used in genetic engineering of plants to either delay or accelerate plastid development and, as a consequence, in improving yield-related traits. The invention is thus aimed at targeting and using SP1/SP1 in different ways, including altering expression of a SP1 gene in a plant, to alter or control plastid development. In summary and in accordance with the various aspects of the invention, altering the expression of a SP1 nucleic acid in a plant (i.e. increasing or decreasing gene expression) or altering the activity of a SP1 protein in a plant (i.e. increasing or decreasing activity) delays or accelerates plastid development, in particular transition from one type of plastid to another. This elicits a beneficial phenotype, such as delaying senescence (‘stay-green’ trait), increasing seedling survival, growth and emergence, increasing starch content or grain size or delaying/accelerating fruit ripening. For example, accelerating the transition of etioplasts to chloroplasts in seedlings by increasing SP1 expression or SP1 activity increases seedling survival, growth and emergence. Furthermore, delaying or inhibiting the transition of chloroplasts to gerontoplasts by inhibiting or reducing SP1 expression or SP1 activity produces a ‘stay-green’ effect. The transition of proplastids to amyloplasts which is involved in controlling starch content or grain size may be increased by increasing SP1 expression or SP1 activity. Fruit ripening is delayed by inhibiting or reducing SP1 expression or SP1 activity as this inhibits the transition of chloroplasts to chromoplasts.

In one aspect, the invention relates to a plant wherein the expression of a SP1 nucleic acid in said plant or the presence or activity of a SP1 polypeptide is increased or decreased. The invention also relates to a transgenic plant which has altered expression of SP1 as it either may comprise a nucleic acid construct which may comprise a SP1 nucleic acid operably linked to a regulatory sequence which directs enhanced or tissue-specific expression of SP1 or has reduced expression of the endogenous SP1 gene. In another aspect, the invention relates to a plant wherein the activity of the SP1 protein has been modified, for example by mutagenesis, and which expresses a mutant SP1 peptide. Related methods and uses as set out below are also within the scope of the invention.

Surprisingly, the inventor has also shown that SP1 is involved in stress tolerance and when overexpressed in a plant can confer increased tolerance to abiotic stress, including salinity, osmotic and oxidative stress.

Thus, in a first aspect, the invention relates to a transgenic plant cell, plant or a part thereof characterised in that
a) the expression of a nucleic acid which may comprise SEQ ID NO. 1 or 2 and encoding a SP1 peptide which may comprise SEQ ID NO. 3, a functional homologue or variant thereof is altered or
b) the activity of a SP1 peptide is altered and said plant expresses a nucleic acid which may comprise a mutant SEQ ID NO. 1 or 2 and encoding a mutant SP1 peptide, a functional homologue or variant thereof which carries a mutation in the RING domain or another domain, for example the IMS domain, wherein the mutation is not as disclosed in reference 31.

The invention also relates to a vector which may comprise a SP1 nucleic acid as defined in SEQ ID NO. 1, 2, 7, 8, 10 or 11 or a functional variant or homologue thereof.

The invention also relates to a method for altering plastid development in a transgenic plant cell, plant or a part thereof or increasing yield of a plant which may comprise
d) altering the expression of a nucleic acid which may comprise SEQ ID NO. 1 or 2 and encoding a SP1 peptide which may comprise SEQ ID NO. 3, a functional homologue or variant thereof or
e) altering the activity of a SP1 peptide which may comprise SEQ ID NO. 3 which may comprise expressing in a plant a nucleic acid which may comprise a mutant SEQ ID NO. 1 or 2 and encoding a mutant SP1 peptide, a functional homologue or variant thereof which carries a mutation in the RING domain.

The invention also relates to a method for improving seedling emergence and survival which may comprise introducing and expressing in a plant a nucleic acid construct which may comprise SEQ ID NO. 1 or 2 and encoding a SP1 peptide which may comprise SEQ ID NO. 3, a functional homolog or variant thereof.

The invention also relates to a method for altering fruit ripening in a transgenic plant cell, plant or a part thereof which may comprise
a) altering the expression of a nucleic acid which may comprise SEQ ID NO. 1 or 2 and encoding a SP1 peptide which may comprise SEQ ID NO. 3, a functional homologue or variant thereof or
b) altering the activity of a SP1 peptide which may comprise SEQ ID NO. 3, a functional homologue or variant thereof.

The invention also relates to a method for making a transgenic plant with altered plastid development which may comprise
a) altering the expression of a nucleic acid which may comprise SEQ ID NO. 1 or 2 and encoding a SP1 peptide which may comprise SEQ ID NO. 2, a functional homologue or variant thereof or
b) altering the activity of a SP1 peptide which may comprise SEQ ID NO. 2, a functional homologue or variant thereof.

The invention also relates to a use of a nucleic acid which may comprise SEQ ID NO. 1 or 2 and encoding a SP1 peptide which may comprise SEQ ID NO. 3 or a nucleic acid encoding a mutant SP1 protein in altering plastid development and/or increasing stress tolerance, in particular to salinity, osmotic and oxidative stress.

The invention also relates to a method for increasing stress tolerance to one or more of salinity, osmotic stress and/or oxidative stress in a plant cell, plant or part thereof which may comprise introducing and expressing in said plant cell, plant or part thereof a nucleic acid construct which may comprise a nucleic acid which may comprise SEQ ID NO. 1 or 2 and encoding a SP1 peptide which may comprise SEQ ID NO. 3, a functional homologue or variant thereof.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. §112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE FIGURES

The following detailed description, given by way of example, but not intended to limit the invention solely to the specific embodiments described, may best be understood in conjunction with the accompanying drawings.

FIG. 1A-G. The sp1 mutation suppresses the phenotype of the atToc33 knockout mutation, ppi1. (A) Plants grown on soil for 30 days. (B) Leaf chlorophyll contents of similar 40-day-old plants. (C) Ultrastructure of typical cotyledon chloroplasts in 10-day-old plants grown in vitro (scale bar, 2 μm). These and other micrographs were used to estimate chloroplast cross-sectional area (D) and thylakoid development (E). (F) Protein import into isolated chloroplasts was measured by quantifying maturation (mat) of in vitro translated (IVT) Rubisco small subunit precursors (pre). (G) Analysis of chloroplast proteins by SDS-PAGE and SYPRO staining, revealing sp1-linked restoration of the three main photosynthetic proteins: Rubisco large (LSU) and small (SSU) subunits; light-harvesting chlorophyll-binding protein (LHCP). All values are means±SEM (N≧4).

FIG. 2A-D. SP1 is located in the chloroplast outer envelope membrane with its RING domain facing the cytosol. (A) SP1 protein map showing transmembrane (TMD), intermembrane space (SP1ims), cytosolic (SP1cyt), and RING finger (RNF) domains. (B) Localization of SP1-YFP to chloroplast envelopes (top) depended on the transmembrane domains, as revealed by a double-deletion mutant (bottom) (scale bar, 10 μm). (C) Radiolabelled SP1 in isolated chloroplasts was located in the membrane pellet (P) fraction after high-pH washing, in contrast with imported mature SSU which was in the soluble (S) fraction. Endogenous markers partitioned as expected (Coomassie stain; bottom). (D) Radiolabelled SP1 and C-terminally-tagged SP1-HA-FLAG were imported into chloroplasts prior to their treatment with thermolysin (Th), trypsin (Tryp), thermolysin plus Triton X-100 (Th/TX), or buffer lacking protease (Mock). Phosphor-imaging revealed protease sensitivity and protected fragments (of sizes not influenced by the C-terminal tag) consistent with outer membrane localization and the topology shown in (A). Immunoblot analysis of three endogenous markers confirmed efficacy of the treatments.

FIG. 3A-G. SP1 associates with TOC complexes and targets TOC components for UPS-mediated degradation via ubiquitination. (A) Immunoblot analysis of total leaf protein from different genotypes, including SP1 overexpressors (OX). Plasma membrane H+-ATPase, PMA2, acted as a loading control. Bars show means±SEM (N=4-6). (B) Co-immunoprecipitation (IP) of TOC components with HA-tagged SP1 from protoplast extracts. Cells were transfected with an SP1-HA construct or empty vector (v). (C) In vitro pull-down of radiolabelled TOC components (or domains) with GST-SP1 baits. (D) In vitro ubiquitination of radiolabelled TOC components (but not atToc159G) by recombinant GST-SP1 flex. Asterisks indicate a non-specific 48 kD band seen in all translations. Mono-ubiquitinated atToc159G would be expected to migrate near the 50 kD marker. (E) Ubiquitination of atToc33 as in (D) using free and HA-tagged ubiquitin (8.5 and 9.4 kD, respectively). (F) Immunoprecipitation of TOC components with FLAG-tagged ubiquitin from transfected protoplasts. (G) Immunoprecipitation under denaturing conditions of FLAG-ubiquitin with atToc33; a control IP utilized excess anti-atTic110. IgG heavy chain (hc) is shown. Ubiquitinated species (Ub) are indicated (D-G).

FIG. 4A-G. SP1 is important for developmental processes that require reorganization of the plastid proteome. (A-E) De-etiolation of seedlings grown in darkness for 6 (A-D) or 5 (E) days, upon transferral to continuous light. (A, B) Cotyledons of typical plants, and survival rates, after two days' illumination. (C, D) Ultrastructure of typical cotyledon plastids after 0, 6 and 24 h illumination (scale bar, 2 μm), and proportion of plastids at each of three progressively more advanced developmental stages (see Methods) after 6 h illumination. (E) Chlorophyll contents after 16 h illumination. (F, G) Senescence of leaves induced by covering with aluminium foil. (F) Typical control (uncovered) and senescent (covered) leaves. (G) Photochemical efficiency of photosystem II (Fv/Fm) was measured to estimate the extent of senescence. All values are means±SEM (N=3-9).

FIG. 5A-C. Overexpression of SP1 enhances the phenotypes of TOC mutants. The SP1 CDS was cloned downstream of the strong, constitutive CaMV 35S promoter in the pB2GW7 binary vector, and then the resultant 35S:SP1 construct was used to stably transform ppi1 plants. Approximately 12 transformants were identified, and from these, representative, single-locus lines were selected for analysis based on segregation of the T-DNA-borne antibiotic-resistance marker, SP1 mRNA expression, and phenotype analysis. The selected transformants are also shown in FIG. 3A (#1 and #5), and in FIGS. 3F and 4. (A, B) Phenotypic analysis of the selected 35S:SP1 transformants. Plants were grown under standard conditions on soil for 25 days before photography (A). Chlorophyll was measured in the leaves of plants of a similar age using a Konica-Minolta SPAD-502 meter (B). Meter values were converted to chlorophyll values (on a per fresh weight basis) using a verified calibration equation. Values shown are means (±SEM) derived from over 25 measurements per genotype. The toc75-III-3 lines were generated by crossing with the 35S:SP1 #1 ppi1 line. (C) Analysis of the expression of the 35S:SP1 transgenes in the selected transformants by RT-PCR. Total RNA samples isolated from 25-day-old plants were analysed by RT-PCR using gene-specific primers for SP1 and the reference gene eIF4E1 (table 1). Wild-type genomic DNA (gDNA) was similarly analysed as a control. Amplifications employed a limited number of cycles to avoid saturation, and products were analysed by agarose gel electrophoresis. Amplicon sizes are indicated to the right of the gel images.

FIG. 6A-F. Importance of the RING domain for SP1 functionality. A mutation affecting a critical residue of the SP1 RING domain (C330A) was introduced into the SP1 CDS by PCR-based mutagenesis. The resulting mutant sequence was then analysed for its ability to complement the sp1-3 mutation in vivo (A-D), and for biochemical activity in vitro (E, F). (A-D) The C330A mutant sequence was cloned downstream of the CaMV 35S promoter in the pB2GW7 binary vector. The resulting 35S:SP1-C330A binary construct (together with the non-mutant 35S:SP1 control construct) was then used to stably transform sp1-3 ppi1 plants. Approximately 12 transformants were identified for each construct, and from these representative, single-locus lines were selected based on segregation of the T-DNA-borne antibiotic-resistance marker, SP1 mRNA expression, and phenotype analysis. (A, B) Phenotypic analysis of the selected 35S:SP1 and 35S:SP1-C330A transformants. Plants were grown under standard conditions on soil for 25 days before photography (A). Chlorophyll was measured in the leaves of plants of a similar age using a Konica-Minolta SPAD-502 meter (B). Meter values were converted to chlorophyll values (on a per fresh weight basis) using a verified calibration equation. Values shown are means (±SEM) derived from ˜20 measurements per genotype. (C, D) Analysis of the expression of the 35S:SP1 and 35S:SP1-C330A transgenes in the selected transformants by semi-quantitative RT-PCR. Total RNA samples isolated from 25-day-old plants were analysed by RT-PCR using gene-specific primers for SP1 and the reference gene eIF4E1 (table S1). Amplifications employed a limited number of cycles to avoid saturation, and products were analysed by agarose gel electrophoresis. Amplicon sizes are indicated to the right of the gel images (C). Amplicons from three independent experiments were quantified using Aida software, and the data were used calculate means±SEM (D). The results clearly indicated that the failure of the 35S:SP1-C330A construct to complement the sp1 mutation (A, B) could not be explained by poor mRNA expression. (E, F) Analysis of SP1 auto-ubiquitination activity in vitro. Various different forms of the SP1 protein were expressed in bacteria using the pDest-565 expression vector, and purified using the added N-terminal GST tag. First, two engineered forms of SP1 lacking the transmembrane domains (to improve solubility) were tested for self-ubiquitination activity, along with a purified GST control (E, left side). The SP1 cyt variant may comprise the cytosolic domain of SP1 only, whereas the SP1flex protein possesses both the intermembrane space and cytosolic domains joined by a flexible linker (SEQ ID NO: 60) (F). Both proteins displayed robust ubiquitination (as indicated by high molecular weight bands of varying size), but the SP1 cyt protein had higher activity and so it was in this context that the influence of the C330A mutation was tested in a similar biochemical assay (E, right side). Positions of molecular weight markers are indicated at left (sizes in kD).

FIG. 7. Overexpression of SPL2 complements the sp1 mutant.

FIG. 8A-B. Altering expression of solSP1 modifies fruit ripening. (A) solSP1KD plants and solSP1 expression levels; (B) overexpressing solSP1KD plants and solSP1 expression levels

FIG. 9A-I. Structure of SP1. (A) The AtSP1 sequence (SEQ ID NO: 3) showing conserved domains (boxed); (B) Alignment of AtSP1 and homologues in other plants (SEQ ID NOS 3, 32-55, 9 and 56, respectively, in order of appearance).

FIG. 10A-C. SP1 confers salinity tolerance. SP1 overexpression (OX) enhances the greening rate of Arabidopsis seedlings after germination on NaCl plates, whereas sp1 mutations make plants more sensitive to NaCl. Germination then growth was on 150 mM NaCl plates for 10 days. (A) shows pictures of the seedlings. (B) shows % tages of green/germinated plants and germination rates.

FIG. 11A-B. SP1 confers tolerance to osmotic stress. SP1 changes the osmotic stress tolerance of Arabidopsis seedlings. Germination then growth was on 400 mM mannitol plates for 3 weeks, and then the proportion of well-developed plants was calculated. (A) shows pictures of the seedlings. (B) shows % tages of developed plants.

FIG. 12A-D. SP1 confers tolerance to oxidative stress. (A, B, C) SP1 regulates the response of Arabidopsis seedlings to oxidative stress, as shown by paraquat (PQ) treatment, which induces accumulation of reactive oxygen species (ROS). Plants were germinated and grown on 1 μM PQ medium (or a range of PQ concentrations) for 10 days prior to analysis. (D) Diaminobenzidine (DAB) staining indicates that SP1 inhibits ROS accumulation after oxidative stress. 10-day-old plants were treated with 3 μM paraquat for 3 days prior to staining.

FIG. 13A-C. Osmotic stress dynamically affects TOC component levels in a SP1-dependent fashion. Western blot analysis of chloroplast proteins under osmotic stress revealed that: 1) TOC proteins are the fastest at responding to stress amongst other proteins tested; 2) SP1 is involved in this response. 7-day-old plants were treated with 300 mM mannitol for 2 days prior to their analysis.

FIG. 14A-B. SP1 expression in salt cress. The SP1 gene is more highly expressed in salt cress (Thelungiella salsuginea; Shandong ecotype) than in Arabidopsis (Col-0 ecotype), which might be related to its paler phenotype. (A) The SRI mRNA expression level in 12-day-old plants was determined by RT-PCR (normalized to ACTIN); equivalent data for the transcriptional elongation factor EF1a is shown as a house-keeping gene control. (B) Appearance of 12-day-old Arabidopsis and salt cress plants, grown side-by-side under identical conditions.

FIG. 15A-C. Manipulation of SP1 expression in transgenic salt cress plants. Artificial microRNA (amiRNA) knock-down (KD) of SP1 gene expression in transgenic salt cress lines leads to a darker green colour and increased Toc75 protein levels. (A) Appearance of 7-week-old transgenic plants; (B) Western blot analysis of Toc75 protein levels in wild-type and KD salt cress plants; Tic110, a protein of the plastid inner envelope membrane, is shown as control. The graph shows quantification of the Western blotting results. (C) Relative protein level.

### DETAILED DESCRIPTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The practice of the present invention will employ, unless otherwise indicated, conventional techniques of botany, microbiology, tissue culture, molecular biology, chemistry, biochemistry and recombinant DNA technology, bioinformatics which are within the skill of the art. Such techniques are explained fully in the literature.

As used herein, the words “nucleic acid”, “nucleic acid sequence”, “nucleotide”, “nucleic acid molecule” or “polynucleotide” are intended to include DNA molecules (e.g., cDNA or genomic DNA), RNA molecules (e.g., mRNA), naturally occurring, mutated, synthetic DNA or RNA molecules, and analogues of the DNA or RNA generated using nucleotide analogues. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene. The term “gene” or “gene sequence” is used broadly to refer to a DNA nucleic acid associated with a biological function. Thus, genes may include introns and exons as in the genomic sequence, or may comprise only a coding sequence as in cDNAs, and/or may include cDNAs in combination with regulatory sequences.

The terms “peptide”, “polypeptide” and “protein” are used interchangeably herein and refer to amino acids in a polymeric form of any length, linked together by peptide bonds.

For the purposes of the invention, “transgenic”, “transgene” or “recombinant” means with regard to, for example, a nucleic acid sequence, an expression cassette, gene construct or a vector which may comprise the nucleic acid sequence or an organism transformed with the nucleic acid sequences, expression cassettes or vectors according to the invention, all those constructions brought about by recombinant methods in which either
a) the nucleic acid sequences encoding proteins useful in the methods of the invention, or
b) genetic control sequence(s) which is operably linked with the nucleic acid sequence according to the invention, for example a promoter, or
c) a) and b)

are not located in their natural genetic environment or have been modified by recombinant methods, it being possible for the modification to take the form of, for example, a substitution, addition, deletion, inversion or insertion of one or more nucleotide residues. The natural genetic environment is understood as meaning the natural genomic or chromosomal locus in the original plant or the presence in a genomic library. In the case of a genomic library, the natural genetic environment of the nucleic acid sequence is preferably retained, at least in part. The environment flanks the nucleic acid sequence at least on one side and has a sequence length of at least 50 bp, preferably at least 500 bp, especially preferably at least 1000 bp, most preferably at least 5000 bp. A naturally occurring expression cassette—for example the naturally occurring combination of the natural promoter of the nucleic acid sequences with the corresponding nucleic acid sequence encoding a polypeptide useful in the methods of the present invention, as defined above—becomes a transgenic expression cassette when this expression cassette is modified by non-natural, synthetic (“artificial”) methods such as, for example; mutagenic treatment. Suitable methods are described, for example, in U.S. Pat. No. 5,565,350 or WO 00/15815 both incorporated by reference.

In certain embodiments, a transgenic plant for the purposes of the invention is thus understood as meaning, as above, that the nucleic acids used in the method of the invention are not at their natural locus in the genome of said plant, it being possible for the nucleic acids to be expressed homologously or heterologously. Thus, the plant expresses a transgene. However, as mentioned, in certain embodiments, transgenic also means that, while the nucleic acids according to the different embodiments of the invention are at their natural position in the genome of a plant, the sequence has been modified with regard to the natural sequence, and/or that the regulatory sequences of the natural sequences have been modified, for example by mutagenesis.

Transgenic is preferably understood as meaning the expression of the nucleic acids according to the invention at an unnatural locus in the genome, i.e. homologous or, preferably, heterologous expression of the nucleic acids takes place. According to the invention, the transgene is stably integrated into the plant and the plant is preferably homozygous for the transgene.

The aspects of the invention involve recombination DNA technology and in preferred embodiments exclude embodiments that are solely based on generating plants by traditional breeding methods.

The inventor has characterised AtSP1 and identified AtSP1 homologues and generated transgenic plants expressing a SP1 nucleic acid construct which have improved yield related traits, including altered plastid development and/or altered stress tolerance.

Yield-related traits are traits or features which are related to plant yield. Yield-related traits may comprise one or more of the following non-limitative list of features: early flowering time, yield, biomass, seed yield, seed viability and germination efficiency, seed/grain size, starch content of grain, early vigour, greenness index, increased growth rate, delayed senescence of green tissue. The term “yield” in general means a measurable produce of economic value, typically related to a specified crop, to an area, and to a period of time. Individual plant parts directly contribute to yield based on their number, size and/or weight, or the actual yield is the yield per square meter for a crop and year, which is determined by dividing total production (includes both harvested and appraised production) by planted square metres. The term “yield” of a plant may relate to vegetative biomass (root and/or shoot biomass), to reproductive organs, and/or to propagules (such as seeds) of that plant. Thus, according to the invention, yield may comprise one or more of and can be measured by assessing one or more of: increased seed yield per plant, increased seed filling rate, increased number of filled seeds, increased harvest index, increased viability/germination efficiency, increased number or size of seeds/capsules/pods, increased growth or increased branching, for example inflorescences with more branches, increased biomass or grain fill. Preferably, increased yield may comprise an increased number of grains/seeds/capsules/pods, increased biomass, increased growth, increased number of floral organs and/or increased floral branching. Yield is increased relative to a control plant.

Thus, in a first aspect, the invention relates to a transgenic plant cell, plant or a part thereof characterised in that
a) the expression of a nucleic acid which may comprise SEQ ID NO. 1 or 2 and encoding a SP1 peptide which may comprise SEQ ID NO. 3, a functional homologue or variant thereof is altered or
b) the activity of a SP1 peptide is altered and said plant expresses a nucleic acid which may comprise a mutant SEQ ID NO. 1 or 2 and encoding a mutant SP1 peptide, a functional homologue or variant thereof which carries a mutation in the RING domain.

In one embodiment, expression is increased relative to a control plant. In one embodiment, expression is decreased or inhibited relative to a control plant. The inventor has shown that said plant cell, plant or a part thereof has modified plastid development. The inventor has also shown that a transgenic plant cell, plant or a part thereof characterised in that the expression of a nucleic acid which may comprise SEQ ID NO. 1 or 2 and encoding a SP1 peptide which may comprise SEQ ID NO. 3, a functional homologue or variant thereof has increased stress tolerance.

In another aspect, the invention relates to a method for altering plastid development in a transgenic plant cell, plant or a part thereof which may comprise
a) altering the expression of a nucleic acid which may comprise SEQ ID NO. 1 or 2 and encoding a SP1 peptide which may comprise SEQ ID NO. 3, a functional homologue or variant thereof or
b) altering the activity of a SP1 peptide which may comprise SEQ ID NO. 3 which may comprise expressing a nucleic acid which may comprise a mutant SEQ ID NO. 1 or 2 and encoding a mutant SP1 peptide, a functional homologue or variant thereof which carries a mutation in the RING domain in said plant cell, plant or a part thereof.

The terms altered, changed and modified are used interchangeably herein. According to the various aspects of the invention, expression of a SP1 nucleic acid or a phenotypic trait of the plant (such as altered plastid development or improved stress tolerance) is measured or assessed and compared to a control plant. A control plant as used herein according to all of the aspects of the invention is a plant which has not been modified according to the methods of the invention. Accordingly, the control plant has not been genetically modified to alter either expression of a nucleic acid encoding SP1 or activity of a SP1 peptide as described herein. In one embodiment, the control plant is a wild type plant. In another embodiment, the control plant is a transgenic plant that does not have altered expression of SP1 or altered activity of a SP1 peptide, but expresses a transgene that does not comprise a SP1 nucleic acid. In another embodiment, the control plant carries the expression vector only or carries a mutant SP1 gene expressing a non-functional SP1 peptide. The control plant is typically of the same plant species, preferably having the same genetic background as the modified plant.

According to the various aspects of the invention, in one embodiment, the SP1 nucleic acid may comprise or may consist of SEQ ID NO. 1 or 2. A nucleic acid which may comprise or may consist of SEQ ID NO. 1 or 2 encodes a SP1 peptide which may comprise or may consist of SEQ ID NO. 3, a functional homologue or variant thereof. In one embodiment, the nucleic acid may comprise or may consist of SEQ ID NO. 1 or 2 encoding a SP1 peptide comprising or consisting of SEQ ID NO. 3. SEQ ID NO. 1 designates the AtSP1 genomic sequence; SEQ ID NO. 2 designates the AtSP1 cDNA sequence. According to the invention, a nucleic acid used according to the various aspects of the invention may therefore be the isolated genomic or the cDNA SP1 sequence, a functional homologue or variant thereof. In one embodiment, the SP1 nucleic acid is cDNA.

The term “functional variant” as used herein, for example with reference to SEQ ID No: 1, 2 or 3, refers to a variant gene or peptide sequence or part of the gene or peptide sequence which retains the biological function of the full non-variant SP1 sequence, for example confers altered plastid development when expressed in a transgenic plant. A functional variant also may comprise a variant of the gene of interest encoding a peptide which has sequence alterations that do not affect function of the resulting protein, for example in non-conserved residues. Also encompassed is a variant that is substantially identical, i.e. has only some sequence variations, for example in non-conserved residues, to the wild type sequences as shown herein and is biologically active, for example complements the A. thaliana sp1 mutant.

Thus, it is understood, as those skilled in the art will appreciate, that the aspects of the invention, including the methods and uses, encompass not only a SP1 nucleic acid, for example a nucleic acid sequence which may comprise or may consist of SEQ ID No: 1 or SEQ ID No: 2, a polypeptide which may comprise or may consist of SEQ ID No: 3, or homologues thereof, but also functional variants of AtSP1 or homologues thereof that do not affect the biological activity and function of the resulting protein. Alterations in a nucleic acid sequence which result in the production of a different amino acid at a given site that do however not affect the functional properties of the encoded polypeptide, are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also produce a functionally equivalent product. Each of the proposed modifications is well within the routine skill in the art, as is determination of retention of biological activity of the encoded products.

Generally, variants of a particular SP1 nucleotide sequence of the invention will have at least about 75%, 76%, 77%, 78%, 79%, 80%, 81%, 82%, 83%, 84%, 85%, 86%, 87%, 88%, 89%, 90%, 92%, 94%, 95%, 96%, 97%, 98% or 99% or more sequence identity to that particular non-variant SP1 nucleotide sequence, for example SEQ ID NO. 1 or 2 as determined by sequence alignment programs described elsewhere herein.

Also, the various aspects of the invention the aspects of the invention, including the methods and uses, encompass not only a SP1 nucleic acid or peptide, but also a fragment or part thereof. By “fragment” or “part” is intended a portion of the nucleotide sequence or a portion of the amino acid sequence and hence of the protein encoded thereby. Fragments of a nucleotide sequence may encode protein fragments that retain the biological activity of the native protein.

The term homologue as used herein also designates an SP1 orthologue from other plant species. A homologue of AtSP1 polypeptide has, in increasing order of preference, at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the amino acid represented by SEQ ID NO: 3. Preferably, overall sequence identity is more than 70% or more than 73%. Preferably, overall sequence identity is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, most preferably 90%, 91%, 92%, 93%, 94%, 95%, 96%, 97%, 98%, or at least 99%. In another embodiment, the homologue of a At SP1 nucleic acid sequence has, in increasing order of preference, at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the nucleic acid represented by SEQ ID NO: 1 or 2. Preferably, overall sequence identity is more than 70% or more than 73%. Preferably, overall sequence identity is at least 70%, 71%, 72%, 73%, 74%, 75%, 76%, 77%, 78%, 79%, 80%, 81%, 82%, 83%, 84%, 85%, 86%, 87%, 88%, 89%; 90%, 91%, 92%, 93%, 94%, 95%, 96%, 97%, 98%, or 99%, most preferably 90%, 91%, 92%, 93%, 94%, 95%, 96%, 97%, 98%, or at least 99%. The overall sequence identity is determined using a global alignment algorithm known in the art, such as the Needleman Wunsch algorithm in the program GAP (GCG Wisconsin Package, Accelrys).

In one embodiment of the plants, methods and uses described herein, the functional homologue is SPL2, for example AtSPL2 (see SEQ ID NO. 7, 8 or 9) or SPL2 in another plant. As shown in the examples, AtSPL2—like AtSP1—complements the Arabidopsis sp1 mutant. In one embodiment of the plants, methods and uses described herein, the functional homologue is as shown in SEQ ID No. 31, 32, 33, 34, 35, 36, 37, 38, 39, 40, 41, 42, 43, 4, 45, 46, 47, 48, 49, 50, 51, 52, 53, 54, 55 or 56.

In one embodiment, an AtSP1 homologue may comprise a RING-type or RNF (RING Finger) domain. RING-type domains are divided into several classes (13). Preferably, this is a class C3HC4/HCa RING domain with the consensus sequence:

(SEQ ID NO: 59)
C-X₁₋₂-C-X_{10-16,20/28}-C-X₁₋₂X-H-X₁₋₂-X-C-X₂-C-X₃-X-X₆₋₃₀-C-
P-X-C-Xₙ

Wherein n designates 0-20 amino acids

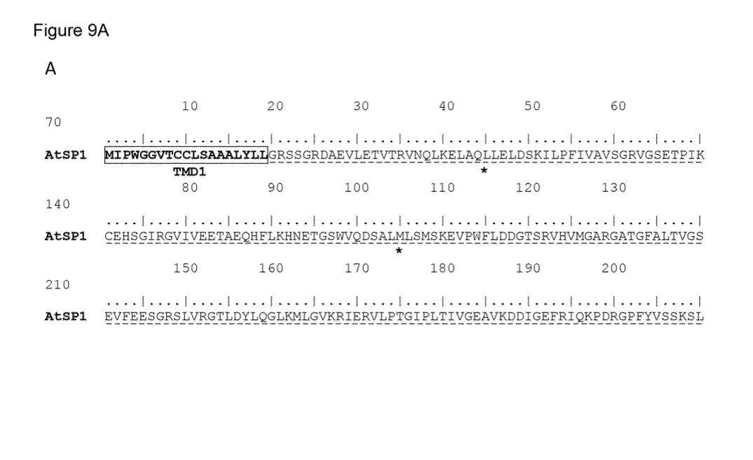

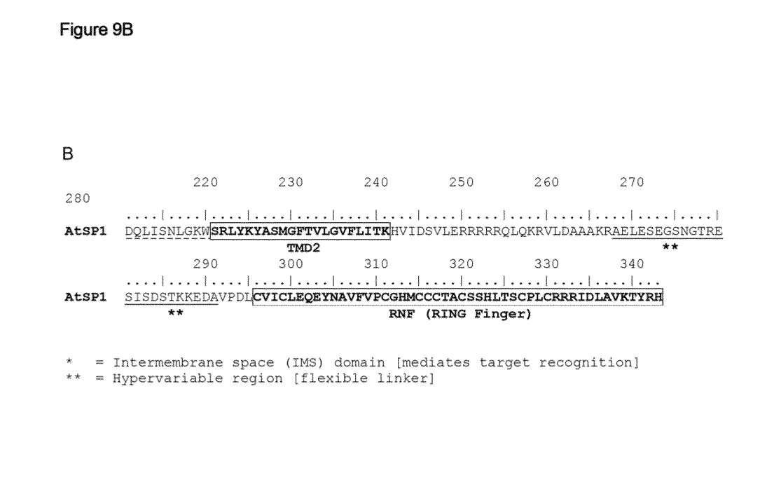

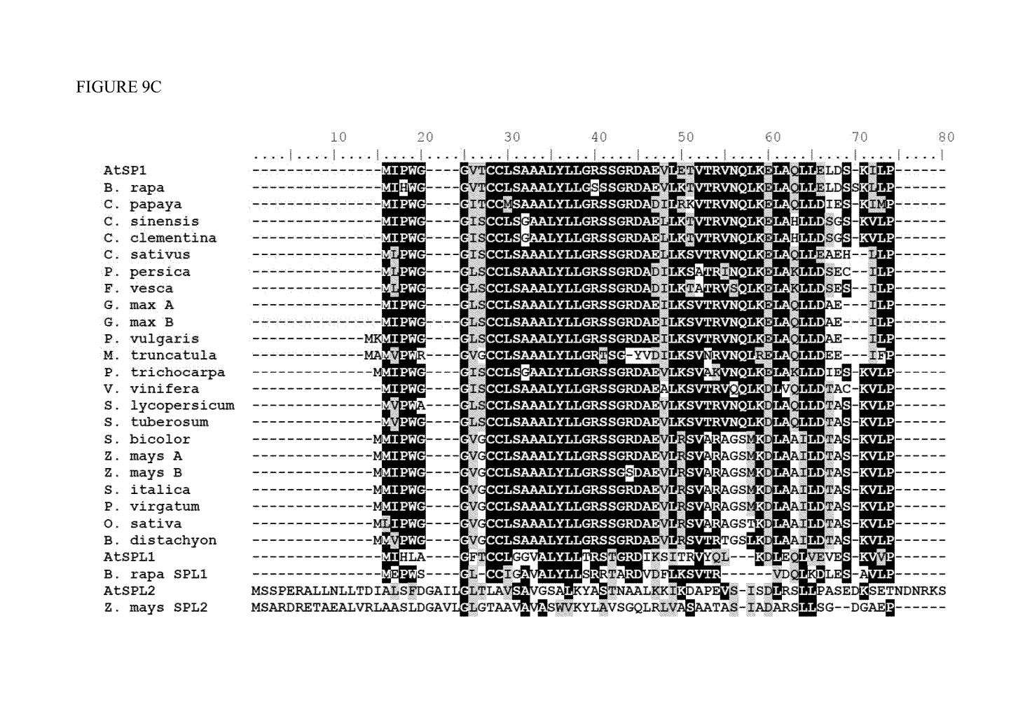

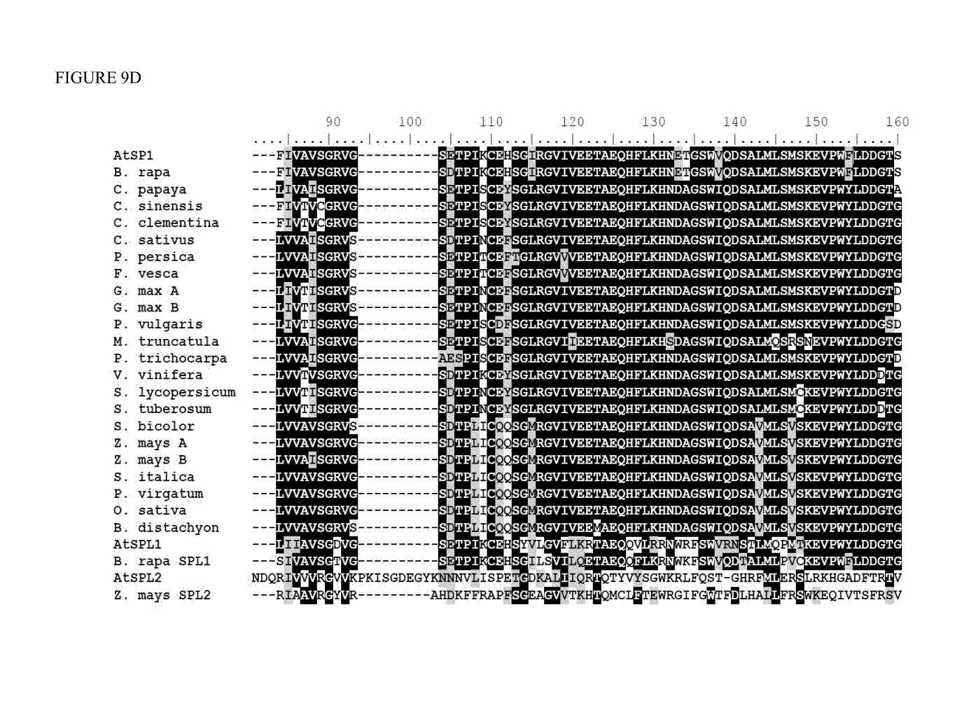

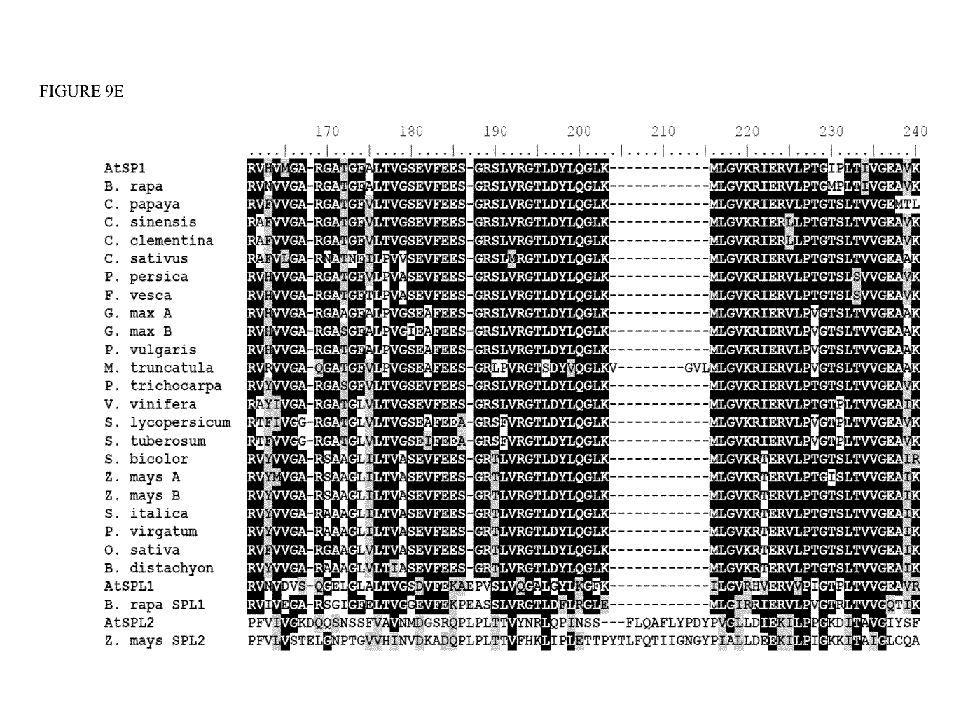

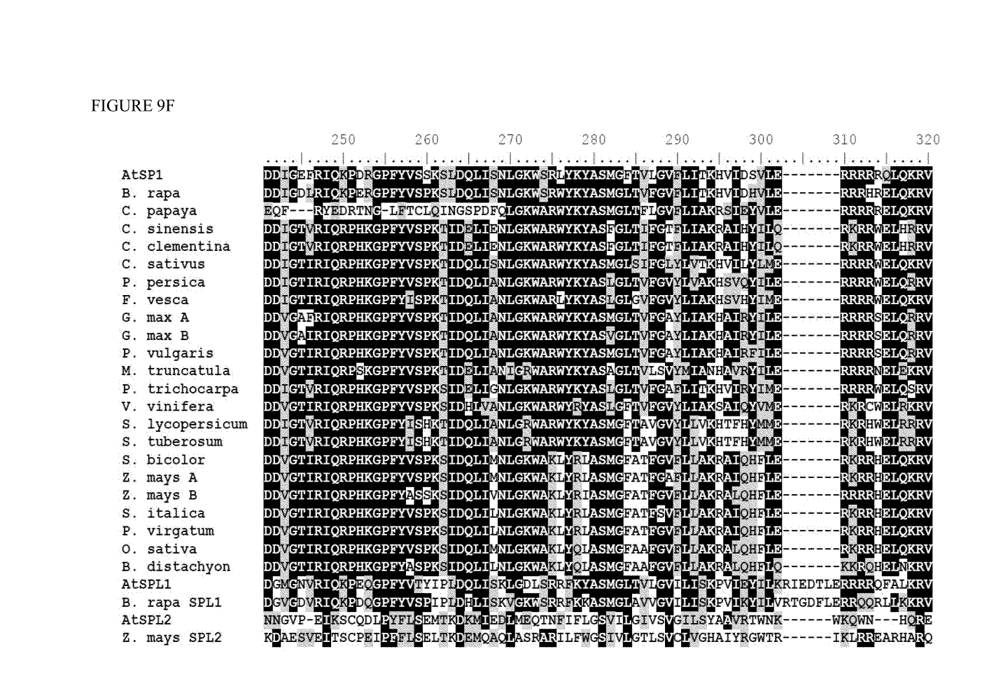

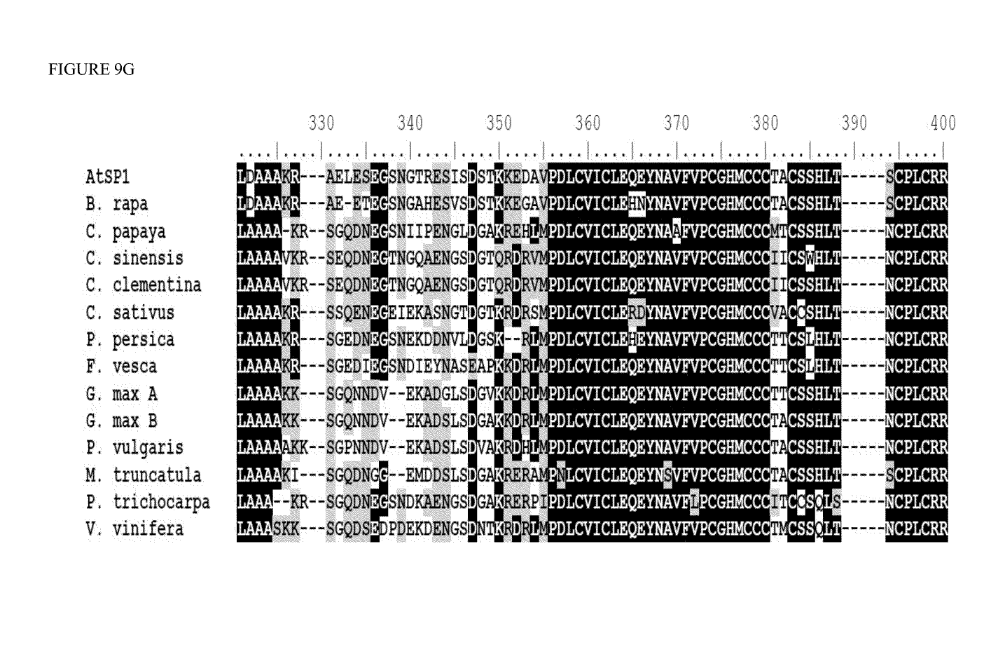

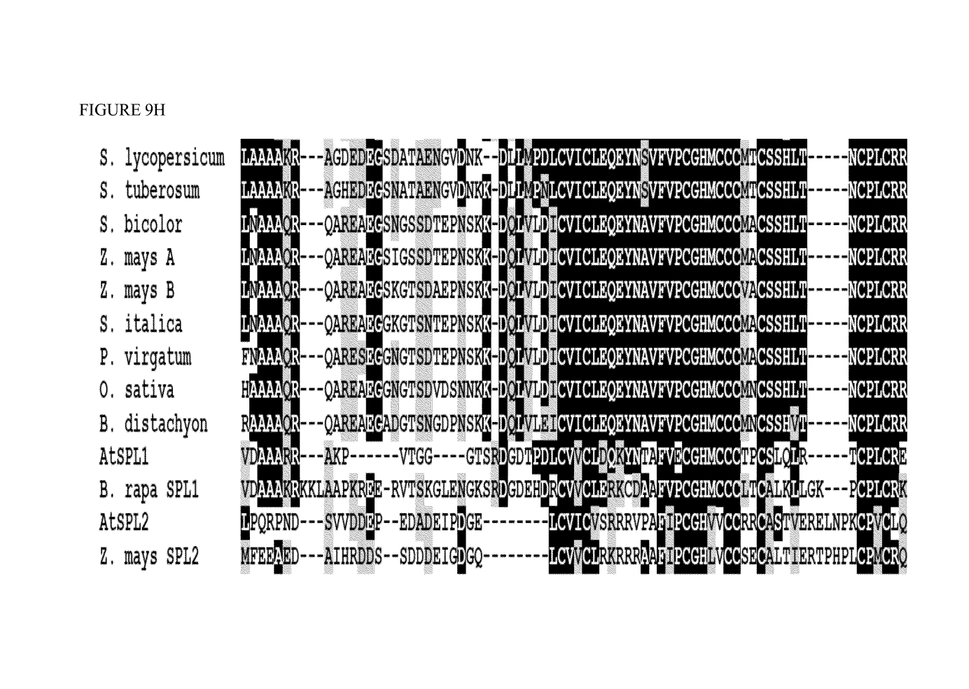

The RNF domain has highly conserved C and H residues which are metal ligands. These are found at positions 1, 4, 16, 22, 24, 32 and 35 of the RING domain of AtSP1 as shown in FIG. 9 or at corresponding positions in AtSP1 homologues. An AtSP1 homologue may comprise a RNF domain with 1, 2, 3, 4, 5, 6, or 7 conserved C residues. One of these is a C at position 330 with reference to AtSP1.

In one embodiment, an AtSP1 homologue may comprise a RING domain having the following sequence:

(SEQ ID NO. 4
CVICLEQEYNAVFVPCGHMCCCTACSSHLTSCPLCRRRIDLAVKTYRH;

conserved C residues are underlined) or a domain with at least 80%, for example 90%, 91%, 92%, 93%, 94%, 95%, 96%, 97%, 98%, or at least 99% sequence identity to this domain.

In one embodiment, an AtSP1 homologue further may comprise a TMD1 domain having the following sequence: MIPWGGVTCCLSAAALYLL (SEQ ID NO. 5) or a domain with at least 90%, 91%, 92%, 93%, 94%, 95%, 96%, 97%, 98%, or at least 99% sequence identity to this domain and a TMD2 domain having the following sequence: SRLYKYASMGFTVLGVFLITK (SEQ ID NO. 6) or a domain with at least 90%, 91%, 92%, 93%, 94%, 95%, 96%, 97%, 98%, or at least 99% sequence identity to this domain.

The structure of the AtSP1 peptide is shown in FIG. 9. The peptide also includes an intermembrane space (IMS) domain which mediates target recognition and a variable region.

SP1 peptides may comprise at least one conserved domain. Preferably, SP1 peptides may comprise a characteristic TMD-IMS-TMD-RNF arrangement.

Suitable homologues can be identified by sequence comparisons and identifications of conserved domains. There are predictors in the art that can be used to identify such sequences. The function of the homologue can be identified as described herein and a skilled person would thus be able to confirm the function when expressed in a plant. Thus, one of skill in the art will recognize that analogous amino acid substitutions listed above with reference to SEQ ID No: 3 can be made in SP1 from other plants by aligning the SP1 receptor polypeptide sequence to be mutated with the AtSP1 polypeptide sequence as set forth in SEQ ID NO: 3.

Thus, the nucleotide sequences of the invention and described herein can be used to isolate corresponding sequences from other organisms, particularly other plants, for example crop plants. In this manner, methods such as PCR, hybridization, and the like can be used to identify such sequences based on their sequence homology to the sequences described herein. Topology of the sequences and the characteristic TMD-IMS-TMD-RNF arrangement can also be considered when identifying and isolating SP1 homologues. Sequences may be isolated based on their sequence identity to the entire sequence or to fragments thereof. In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen plant. The hybridization probes may be genomic DNA fragments, cDNA fragments, RNA fragments, or other oligonucleotides, and may be labelled with a detectable group, or any other detectable marker. Thus, for example, probes for hybridization can be made by labelling synthetic oligonucleotides based on the ABA-associated sequences of the invention. Methods for preparation of probes for hybridization and for construction of cDNA and genomic libraries are generally known in the art and are disclosed in Sambrook, et al., (1989) Molecular Cloning: A Library Manual (2d ed., Cold Spring Harbor Laboratory Press, Plainview, N.Y.).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Duration of hybridization is generally less than about 24 hours, usually about 4 to 12. Stringent conditions may also be achieved with the addition of destabilizing agents such as formamide.

Preferred homologues of AtSP1 peptides are SP1 peptides from crop plants, for example cereal crops. In one embodiment, preferred homologues include SP1 in maize, rice, wheat, sorghum, sugar cane, oilseed rape (canola), soybean, cotton, potato, tomato (solSP1, accession No. Solyc06g084360.1.1, SEQ ID No. 10, 11 and 31), tobacco, grape, barley, pea, bean, field bean or other legumes, lettuce, sunflower, alfalfa, sugar beet, broccoli or other vegetable brassicas or poplar. Preferred homologues and their peptide sequences are also shown in FIG. 9.

A plant according to the various aspects of the invention, including the transgenic plants, methods and uses described herein may be a monocot or a dicot plant.

A dicot plant may be selected from the families including, but not limited to Asteraceae, Brassicaceae (e.g. Brassica napus), Chenopodiaceae, Cucurbitaceae, Leguminosae (Caesalpiniaceae, Aesalpiniaceae Mimosaceae, Papilionaceae or Fabaceae), Malvaceae, Rosaceae or Solanaceae. For example, the plant may be selected from lettuce, sunflower, Arabidopsis, broccoli, spinach, water melon, squash, cabbage, tomato, potato, yam, capsicum, tobacco, cotton, okra, apple, rose, strawberry, alfalfa, bean, soybean, field (fava) bean, pea, lentil, peanut, chickpea, apricots, pears, peach, grape vine, bell pepper, chilli or citrus species. In one embodiment, the plant is oilseed rape.

Also included are biofuel and bioenergy crops such as rape/canola, sugar cane, sweet sorghum, Panicum virgatum (switchgrass), linseed, lupin and willow, poplar, poplar hybrids, Miscanthus or gymnosperms, such as loblolly pine. Also included are crops for silage (maize), grazing or fodder (grasses, clover, sanfoin, alfalfa), fibres (e.g. cotton, flax), building materials (e.g. pine, oak), pulping (e.g. poplar), feeder stocks for the chemical industry (e.g. high erucic acid oil seed rape, linseed) and for amenity purposes (e.g. turf grasses for golf courses), ornamentals for public and private gardens (e.g. snapdragon, petunia, roses, geranium, Nicotiana sp.) and plants and cut flowers for the home (African violets, Begonias, chrysanthemums, geraniums, Coleus spider plants, Dracaena, rubber plant).

A monocot plant may, for example, be selected from the families Arecaceae, Amaryllidaceae or Poaceae. For example, the plant may be a cereal crop, such as wheat, rice, barley, maize, oat, sorghum, rye, millet, buckwheat, turf grass, Italian rye grass, sugarcane or Festuca species, or a crop such as onion, leek, yam or banana.

Preferably, the plant is a crop plant. By crop plant is meant any plant which is grown on a commercial scale for human or animal consumption or use. In a preferred embodiment, the plant is a cereal or legume.

Most preferred plants are maize, rice, wheat, oilseed rape/canola, sorghum, soybean, sunflower, alfalfa, potato, tomato, tobacco, grape, barley, pea, bean, field bean, lettuce, cotton, sugar cane, sugar beet, broccoli or other vegetable brassicas or poplar.

The term “plant” as used herein encompasses whole plants, ancestors and progeny of the plants and plant parts, including seeds, fruit, shoots, stems, leaves, roots (including tubers), flowers, and tissues and organs, wherein each of the aforementioned may comprise the gene/nucleic acid of interest. The term “plant” also encompasses plant cells, suspension cultures, callus tissue, embryos, meristematic regions, gametophytes, sporophytes, pollen and microspores, again wherein each of the aforementioned may comprise the gene/nucleic acid of interest.

The term plastid refers to any plant plastid, including etioplasts, chloroplasts, amyloplasts, elaioplasts, chromoplasts or gerontoplasts. Preferably, the plastid is a chloroplast. Preferably, the development is the transition from one type of plastid to another. Preferably, plastid development is chloroplast development and more preferably refers to the transition of an etioplast into a chloroplast or the transition of a chloroplast into a gerontoplast. In another embodiment, plastid development is the transition from a chloroplast into a chromoplast.

As explained above, a first aspect of the invention relates to a transgenic plant cell, plant or a part thereof characterised in that the expression of a nucleic acid which may comprise SEQ ID NO. 1 or 2 and encoding a SP1 peptide which may comprise SEQ ID NO. 3, a functional homologue or variant thereof is altered.

In one embodiment, the level of SP1 gene expression is increased. For example, the overall level of SP1 gene expression is increased or the level of SP1 gene expression in a particular plant organ or cell type is increased.

In one embodiment, the expression of a nucleic acid which may comprise SEQ ID NO. 1 or 2 and encoding a SP1 peptide which may comprise SEQ ID NO. 3, a functional homologue or variant thereof is increased because the transgenic plant cell, plant or a part thereof of the first aspect of the invention expresses a nucleic acid construct which may comprise SEQ ID NO. 1 or 2 and encoding a SP1 peptide which may comprise SEQ ID NO. 3, a functional homologue or variant thereof. Thus, the plant includes in its genome an exogenous nucleic acid construct which may comprise SEQ ID NO. 1 or 2 which directs the expression of the SP1 peptide which may comprise SEQ ID NO. 3, a functional homologue or variant thereof.

Plastid development is accelerated in said plant cell, plant or a part thereof. Furthermore, stress tolerance is increased in the plant. In one embodiment, the nucleic acid construct further may comprise a regulatory sequence.

According to the plants, methods and uses described herein, the term “regulatory element” is used interchangeably herein with “control sequence” and “promoter” and all terms are to be taken in a broad context to refer to regulatory nucleic acid sequences capable of effecting expression of the sequences to which they are ligated. The term “promoter” typically refers to a nucleic acid control sequence located upstream from the transcriptional start of a gene and which is involved binding of RNA polymerase and other proteins, thereby directing transcription of an operably linked nucleic acid. Encompassed by the aforementioned terms are transcriptional regulatory sequences derived from a classical eukaryotic genomic gene (including the TATA box which is required for accurate transcription initiation, with or without a CCAAT box sequence) and additional regulatory elements (i.e. upstream activating sequences, enhancers and silencers) which alter gene expression in response to developmental and/or external stimuli, or in a tissue-specific manner. Also included within the term is a transcriptional regulatory sequence of a classical prokaryotic gene, in which case it may include a −35 box sequence and/or −10 box transcriptional regulatory sequences.

The term “regulatory element” also encompasses a synthetic fusion molecule or derivative that confers, activates or enhances expression of a nucleic acid molecule in a cell, tissue or organ.

A “plant promoter” may comprise regulatory elements, which mediate the expression of a coding sequence segment in plant cells. Accordingly, a plant promoter need not be of plant origin, but may originate from viruses or micro-organisms, for example from viruses which attack plant cells. The “plant promoter” can also originate from a plant cell, e.g. from the plant which is transformed with the nucleic acid sequence to be expressed in the inventive process and described herein. This also applies to other “plant” regulatory signals, such as “plant” terminators. The promoters upstream of the nucleotide sequences useful in the methods of the present invention can be modified by one or more nucleotide substitution(s), insertion(s) and/or deletion(s) without interfering with the functionality or activity of either the promoters, the open reading frame (ORF) or the 3′-regulatory region such as terminators or other 3′ regulatory regions which are located away from the ORF. It is furthermore possible that the activity of the promoters is increased by modification of their sequence, or that they are replaced completely by more active promoters, even promoters from heterologous organisms. For expression in plants, the nucleic acid molecule is, as described above, preferably linked operably to or may comprise a suitable promoter which expresses the gene at the right point in time and with the required spatial expression pattern. For the identification of functionally equivalent promoters, the promoter strength and/or expression pattern of a candidate promoter may be analysed for example by operably linking the promoter to a reporter gene and assaying the expression level and pattern of the reporter gene in various tissues of the plant. Suitable well-known reporter genes are known to the skilled person and include for example beta-glucuronidase or beta-galactosidase.

The term “operably linked” as used herein refers to a functional linkage between the promoter sequence and the gene of interest, such that the promoter sequence is able to initiate transcription of the gene of interest.

For example, the nucleic acid sequence may be expressed using a promoter that drives overexpression. Overexpression according to the invention means that the transgene is expressed at a level that is higher than expression of endogenous counterparts driven by their endogenous promoters. For example, overexpression may be carried out using a strong promoter, such as a constitutive promoter. A “constitutive promoter” refers to a promoter that is transcriptionally active during most, but not necessarily all, phases of growth and development and under most environmental conditions, in at least one cell, tissue or organ. Examples of constitutive promoters include the cauliflower mosaic virus promoter (CaMV35S or 19S), rice actin promoter, maize ubiquitin promoter, rubisco small subunit, maize or alfalfa H3 histone, OCS, SAD1 or 2, GOS2 or any promoter that gives enhanced expression. Alternatively, enhanced or increased expression can be achieved by using transcription or translation enhancers or activators and may incorporate enhancers into the gene to further increase expression. Furthermore, an inducible expression system may be used, where expression is driven by a promoter induced by environmental stress conditions (for example the pepper pathogen-induced membrane protein gene CaPIMPI or promoters that may comprise the dehydration-responsive element (DRE), the promoter of the sunflower HD-Zip protein genes Hahb1 or Hahb4, which is inducible by water stress, high salt concentrations and ABA or a chemically inducible promoter (such as steroid- or ethanol-inducible promoter system). The promoter may also be tissue-specific. The types of promoters listed above are described in the art. Other suitable promoters and inducible systems are also known to the skilled person.

In another embodiment, a promoter specific for seed development (e.g. HaFAD2-1 from sunflower or a seed storage protein promoter, such as zein, glutenin or hordein) or seed maturation (e.g. soybean pm36) may be used, or one specific for seed germination (e.g. barley or wheat alpha-amylase or carboxypeptidase) or a seedling-specific promoter (such as the Pyk10 promoter) may be used. The patatin promoter may be used for tubers.

In another embodiment, a green tissue-specific promoter may be used. For example, a green tissue-specific promoter may be selected from the maize orthophosphate kinase promoter, maize phosphoenolpyruvate carboxylase promoter, rice phosphoenolpyruvate carboxylase promoter, rice small subunit rubisco promoter, rice beta expansin EXBO9 promoter, pigeonpea small subunit rubisco promoter or pea RBS3A promoter.

In one embodiment, the promoter is a constitutive or strong promoter. In a preferred embodiment, the regulatory sequence is an inducible promoter or a stress inducible promoter. The stress inducible promoter is selected from the following non limiting list: the HaHB1 promoter, RD29A (which drives drought inducible expression of DREB1A), the maize rabl7 drought-inducible promoter, P5CS1 (which drives drought inducible expression of the proline biosynthetic enzyme P5CS1), ABA- and drought-inducible promoters of Arabidopsis clade A PP2Cs (ABI1, ABI2, HAB1, PP2CA, HAI1, HAI2 and HAI3) or their corresponding crop orthologues.

In one embodiment, the invention relates to a transgenic plant cell, plant or a part thereof that expresses a nucleic acid construct which may comprise SEQ ID NO. 1 or 2 and encoding a SP1 peptide which may comprise SEQ ID NO. 3, a functional homologue or variant thereof wherein the promoter is a seedling-specific, strong or constitutive promoter and said plant or part thereof is a seed or seedling. The transition from etioplasts to chloroplasts is accelerated in said seedling. This leads to improved seedling survival, growth and emergence. Furthermore, the transition from proplastids to amyloplasts may also be accelerated. This leads to improved starch content and/or increased seed/grain size and thus improved yield.

According to the invention, expression of the SP1 nucleic acid can also be altered by decreasing or inhibiting its expression in a plant using recombinant technology. Thus, in another embodiment, the invention relates to a transgenic plant cell, plant or a part thereof according to claim 1 wherein the expression of a nucleic acid which may comprise SEQ ID NO. 1 or 2 and encoding a SP1 peptide which may comprise SEQ ID NO. 3, a functional homologue or variant thereof is inactivated, repressed or down-regulated. Plastid development is delayed in said plant. For example, expression of the endogenous SP1 nucleic acid which may comprise SEQ ID NO. 1 or 2 and encoding a SP1 peptide which may comprise SEQ ID NO. 3, a functional homologue or variant thereof can be silenced using gene silencing. According to said aspect, a nucleic acid construct which specifically targets SEQ ID NO. 1 or 2 and encoding a SP1 peptide which may comprise SEQ ID NO. 3, a functional homologue or variant thereof is introduced and expressed in said plant as shown in example 2. Said nucleic acid construct is a si RNA, for example a amiRNA.

In a preferred embodiment, said plant part is green tissue, for example a leaf, and the transition from chloroplast to gerontoplast is delayed. In another preferred embodiment, said plant part is a fruit and the transition from chloroplast to chromoplast is delayed. In another embodiment, the promoter is a strong or constitutive promoter or a green tissue-specific promoter.

Making a transgenic plant cell, plant or a part thereof wherein the expression of a nucleic acid which may comprise SEQ ID NO. 1 or 2 and encoding a SP1 peptide which may comprise SEQ ID NO. 3, a functional homologue or variant thereof is inactivated, repressed or down-regulated can be achieved by using gene silencing, for example RNA-mediated gene suppression or RNA silencing. “Gene silencing” is a term generally used to refer to suppression of expression of a gene via sequence-specific interactions that are mediated by RNA molecules. The degree of reduction may be so as to totally abolish production of the encoded gene product, but more usually the abolition of expression is partial, with some degree of expression remaining. The term should not therefore be taken to require complete “silencing” of expression.

Transgenes may be used to suppress endogenous plant genes. This was discovered originally when chalcone synthase transgenes in petunia caused suppression of the endogenous chalcone synthase genes and indicated by easily visible pigmentation changes. Subsequently it has been described how many, if not all plant genes can be “silenced” by transgenes. Gene silencing requires sequence similarity between the transgene and the gene that becomes silenced. This sequence homology may involve promoter regions or coding regions of the silenced target gene. When coding regions are involved, the transgene able to cause gene silencing may have been constructed with a promoter that would transcribe either the sense or the antisense orientation of the coding sequence RNA. It is likely that the various examples of gene silencing involve different mechanisms that are not well understood. In different examples there may be transcriptional or post-transcriptional gene silencing and both may be used according to the methods of the invention.

The mechanisms of gene silencing and their application in genetic engineering, which were first discovered in plants in the early 1990 s and then shown in Caenorhabditis elegans are extensively described in the literature.

RNA-mediated gene suppression or RNA silencing according to the methods of the invention includes co-suppression wherein over-expression of the SP1 sense RNA or mRNA leads to a reduction in the level of expression of the genes concerned. RNAs of the transgene and homologous endogenous gene are co-ordinately suppressed. Other techniques used in the methods of the invention include antisense RNA to reduce transcript levels of the endogenous SP1 gene in a plant. In this method, RNA silencing does not affect the transcription of a gene locus, but only causes sequence-specific degradation of target mRNAs. An “antisense” nucleic acid sequence may comprise a nucleotide sequence that is complementary to a “sense” nucleic acid sequence encoding a SP1 protein, or a part of a SP1 protein, i.e. complementary to the coding strand of a double-stranded cDNA molecule or complementary to an mRNA transcript sequence. The antisense nucleic acid sequence is preferably complementary to the endogenous SP1 gene to be silenced. The complementarity may be located in the “coding region” and/or in the “non-coding region” of a gene. The term “coding region” refers to a region of the nucleotide sequence which may comprise codons that are translated into amino acid residues. The term “non-coding region” refers to 5′ and 3′ sequences that flank the coding region that are transcribed but not translated into amino acids (also referred to as 5′ and 3′ untranslated regions).

Antisense nucleic acid sequences can be designed according to the rules of Watson and Crick base pairing. The antisense nucleic acid sequence may be complementary to the entire SP1 nucleic acid sequence, but may also be an oligonucleotide that is antisense to only a part of the nucleic acid sequence (including the mRNA 5′ and 3′ UTR). For example, the antisense oligonucleotide sequence may be complementary to the region surrounding the translation start site of an mRNA transcript encoding a polypeptide. The length of a suitable antisense oligonucleotide sequence is known in the art and may start from about 50, 45, 40, 35, 30, 25, 20, 15 or 10 nucleotides in length or less. An antisense nucleic acid sequence according to the invention may be constructed using chemical synthesis and enzymatic ligation reactions using methods known in the art. For example, an antisense nucleic acid sequence (e.g., an antisense oligonucleotide sequence) may be chemically synthesized using naturally occurring nucleotides or variously modified nucleotides designed to increase the biological stability of the molecules or to increase the physical stability of the duplex formed between the antisense and sense nucleic acid sequences, e.g., phosphorothioate derivatives and acridine-substituted nucleotides may be used. Examples of modified nucleotides that may be used to generate the antisense nucleic acid sequences are well known in the art. The antisense nucleic acid sequence can be produced biologically using an expression vector into which a nucleic acid sequence has been subcloned in an antisense orientation (i.e., RNA transcribed from the inserted nucleic acid will be of an antisense orientation to a target nucleic acid of interest). Preferably, production of antisense nucleic acid sequences in plants occurs by means of a stably integrated nucleic acid construct which may comprise a promoter, an operably linked antisense oligonucleotide, and a terminator.

The nucleic acid molecules used for silencing in the methods of the invention hybridize with or bind to mRNA transcripts and/or insert into genomic DNA encoding a polypeptide to thereby inhibit expression of the protein, e.g., by inhibiting transcription and/or translation. The hybridization can be by conventional nucleotide complementarity to form a stable duplex, or, for example, in the case of an antisense nucleic acid sequence which binds to DNA duplexes, through specific interactions in the major groove of the double helix. Antisense nucleic acid sequences may be introduced into a plant by transformation or direct injection at a specific tissue site. Alternatively, antisense nucleic acid sequences can be modified to target selected cells and then administered systemically. For example, for systemic administration, antisense nucleic acid sequences can be modified such that they specifically bind to receptors or antigens expressed on a selected cell surface, e.g., by linking the antisense nucleic acid sequence to peptides or antibodies which bind to cell surface receptors or antigens. The antisense nucleic acid sequences can also be delivered to cells using vectors.

RNA interference (RNAi) is another post-transcriptional gene-silencing phenomenon which may be used according to the methods of the invention. This is induced by double-stranded RNA in which mRNA that is homologous to the dsRNA is specifically degraded. It refers to the process of sequence-specific post-transcriptional gene silencing mediated by short interfering RNAs (siRNA). The process of RNAi begins when the enzyme, DICER, encounters dsRNA and chops it into pieces called small-interfering RNAs (siRNA). This enzyme belongs to the RNase III nuclease family. A complex of proteins gathers up these RNA remains and uses their code as a guide to search out and destroy any RNAs in the cell with a matching sequence, such as target mRNA.

Artificial and/or natural microRNAs (miRNAs) may be used to knock out gene expression and/or mRNA translation. MicroRNAs (miRNAs) miRNAs are typically single stranded small RNAs typically 19-24 nucleotides long Most plant miRNAs have perfect or near-perfect complementarity with their target sequences. However, there are natural targets with up to five mismatches. They are processed from longer non-coding RNAs with characteristic fold-back structures by double-strand specific RNases of the Dicer family. Upon processing, they are incorporated in the RNA-induced silencing complex (RISC) by binding to its main component, an Argonaute protein. miRNAs serve as the specificity components of RISC, since they base-pair to target nucleic acids, mostly mRNAs, in the cytoplasm. Subsequent regulatory events include target mRNA cleavage and destruction and/or translational inhibition. Effects of miRNA overexpression are thus often reflected in decreased mRNA levels of target genes. Artificial microRNA (amiRNA) technology has been applied in Arabidopsis thaliana and other plants to efficiently silence target genes of interest. The design principles for amiRNAs have been generalized and integrated into a Web-based tool (http://wmd.weigelworld.org).

An example of an amiRNA targeting SP1 is described in examples 2 or 3.

Thus, according to the various aspects of the invention a plant may be transformed to introduce a RNAi, snRNA, dsRNA, siRNA, miRNA, ta-siRNA, amiRNA or cosuppression molecule that has been designed to target the expression of an SP1 gene and selectively decreases or inhibits the expression of the gene or stability of its transcript. Preferably, the RNAi, snRNA, dsRNA, siRNA, miRNA, amiRNA, ta-siRNA or cosuppression molecule used according to the various aspects of the invention may comprise a fragment of at least 17 nt, preferably 22 to 26 nt and can be designed on the basis of the information shown in SEQ ID No. 1. Guidelines for designing effective siRNAs are known to the skilled person. Briefly, a short fragment of the target gene sequence (e.g., 19-40 nucleotides in length) is chosen as the target sequence of the siRNA of the invention. The short fragment of target gene sequence is a fragment of the target gene mRNA. In preferred embodiments, the criteria for choosing a sequence fragment from the target gene mRNA to be a candidate siRNA molecule include 1) a sequence from the target gene mRNA that is at least 50-100 nucleotides from the 5′ or 3′ end of the native mRNA molecule, 2) a sequence from the target gene mRNA that has a G/C content of between 30% and 70%, most preferably around 50%, 3) a sequence from the target gene mRNA that does not contain repetitive sequences (e.g., AAA, CCC, GGG, TTT, AAAA, CCCC, GGGG, TTTT), 4) a sequence from the target gene mRNA that is accessiblein the mRNA, 5) a sequence from the target gene mRNA that is unique to the target gene, 6) avoids regions within 75 bases of a start codon. The sequence fragment from the target gene mRNA may meet one or more of the criteria identified above. The selected gene is introduced as a nucleotide sequence in a prediction program that takes into account all the variables described above for the design of optimal oligonucleotides. This program scans any mRNA nucleotide sequence for regions susceptible to be targeted by siRNAs. The output of this analysis is a score of possible siRNA oligonucleotides. The highest scores are used to design double stranded RNA oligonucleotides that are typically made by chemical synthesis. In addition to siRNA which is complementary to the mRNA target region, degenerate siRNA sequences may be used to target homologous regions. siRNAs according to the invention can be synthesized by any method known in the art. RNAs are preferably chemically synthesized using appropriately protected ribonucleoside phosphoramidites and a conventional DNA/RNA synthesizer. Additionally, siRNAs can be obtained from commercial RNA oligonucleotide synthesis suppliers.

siRNA molecules according to the aspects of the invention may be double stranded. In one embodiment, double stranded siRNA molecules may comprise blunt ends. In another embodiment, double stranded siRNA molecules may comprise overhanging nucleotides (e.g., 1-5 nucleotide overhangs, preferably 2 nucleotide overhangs). In some embodiments, the siRNA is a short hairpin RNA (shRNA); and the two strands of the siRNA molecule may be connected by a linker region (e.g., a nucleotide linker or a non-nucleotide linker). The siRNAs of the invention may contain one or more modified nucleotides and/or non-phosphodiester linkages. Chemical modifications well known in the art are capable of increasing stability, availability, and/or cell uptake of the siRNA. The skilled person will be aware of other types of chemical modification which may be incorporated into RNA molecules.

In one embodiment, recombinant DNA constructs as described in U.S. Pat. No. 6,635,805, incorporated herein by reference, may be used.

The silencing RNA molecule is introduced into the plant using conventional methods, for example a vector and Agrobacterium-mediated transformation. Stably transformed plants are generated and expression of the SP1 gene compared to a wild type control plant is analysed.

Silencing of the SP1 gene may also be achieved using virus-induced gene silencing.

Thus, in one embodiment of the invention, the plant cell, plant or part thereof expresses a nucleic acid construct which may comprise a RNAi, snRNA, dsRNA, siRNA, miRNA, ta-siRNA, amiRNA or co-suppression molecule that targets the SP1 gene as described herein and reduces expression of the endogenous SP1 gene. A gene is targeted when, for example, the RNAi, snRNA, dsRNA, siRNA, miRNA, ta-siRNA, amiRNA or cosuppression molecule selectively decreases or inhibits the expression of the gene compared to a control plant. Alternatively, a RNAi, snRNA, dsRNA, siRNA, miRNA, ta-siRNA, amiRNA or cosuppression molecule targets SP1 when the RNAi, snRNA, dsRNA, siRNA, miRNA, ta-siRNA, amiRNA or cosuppression molecule hybridises under stringent conditions to the gene transcript. Within the context of the invention, preferably, to specifically target SP1, the RNA may comprise at least the same seed sequence. Thus, any RNA that targets SP1 is preferably identical in positions 2-8 of the antisense strand.

Gene silencing may also occur if there is a mutation on an endogenous gene and/or a mutation on an isolated gene/nucleic acid subsequently introduced into a plant. The reduction or substantial elimination may be caused by a non-functional polypeptide. For example, the polypeptide may bind to various interacting proteins; one or more mutation(s) and/or truncation(s) may therefore provide for a polypeptide that is still able to bind interacting proteins (such as receptor proteins) but that cannot exhibit its normal function (such as signalling ligand).

A further approach to gene silencing is by targeting nucleic acid sequences complementary to the regulatory region of the gene (e.g., the promoter and/or enhancers) to form triple helical structures that prevent transcription of the gene in target cells. Other methods, such as the use of antibodies directed to an endogenous polypeptide for inhibiting its function in planta, or interference in the signalling pathway in which a polypeptide is involved, will be well known to the skilled man. In particular, it can be envisaged that manmade molecules may be useful for inhibiting the biological function of a target polypeptide, or for interfering with the signalling pathway in which the target polypeptide is involved.

In another embodiment of the invention directed to a transgenic plant cell, plant or a part thereof, the transgenic plant cell, plant or a part thereof is characterised in that the activity of a SP1 peptide is altered and said plant expresses a nucleic acid which may comprise a mutant SEQ ID NO. 1 or 2 and encoding a mutant SP1 peptide, a functional homologue or variant thereof which carries a mutation in the RING domain.

The activity can be inactivated or repressed.

In one embodiment, the plant expresses a nucleic acid construct which may comprise a mutant SP1 nucleic acid, for example a mutant of SEQ ID NO. 1 or 2, which encodes a mutant SP1 peptide which carries a mutation in the RING domain which renders the peptide non-functional. In other words, the plant expresses an SP1 transgene that encodes a mutant peptide. The nucleic acid construct preferably may comprise a regulatory sequence. This is described elsewhere and can be a promoter that directs overexpression of SP1 or expression of SP1 in a specific tissue, for example in green tissue such as leaves.

As mentioned above, transgenic according to the invention also means that, while the nucleic acids according to the different embodiments of the invention are at their natural position in the genome of a plant, the sequence has been modified with regard to the natural sequence, and/or that the regulatory sequences of the natural sequences have been modified, for example by mutagenesis.

Thus, in one embodiment, the endogenous SP1 gene has been altered, for example by mutagenesis, to carry mutation in the RING domain.

According to the invention, the mutation in the RING domain may be a substitution, deletion of one or more residue within the RING domain or an insertion into the RING domain. As described above, the RING is preferably a class C3HC4/HCa RING domain with the consensus sequence:

(SEQ ID NO: 59)
C-X₁₋₂-C-X_{10-16,} _{20/28}-C-X₁₋₂X-H-X₁₋₂-X-C-X₂-C-X₃-X-X₆₋₃₀-
C-P-X-C-Xₙ

Wherein n designates 0-20 amino acids

The RNF domain has highly conserved C and H residues which are metal ligands. These are found at positions 1, 4, 16, 22, 24, 32 and 35 in AtSP1 (using the numbering of RING residues in SEQ ID No. 4). In one embodiment, one, or more of these residues in AtSP1 or at corresponding positions in a SP1 homologue is deleted or substituted. The substitution may be with A or another suitable amino acid. A preferred mutation is the substitution of a C at position 330, for example C330A, in AtSP1 or the substitution of a C at a corresponding position in a functional SP1 homologue. As shown in the examples, this leads to loss of function of the protein.

Specifically disclaimed from the scope of the invention are the mutants disclosed in reference 31.

In another aspect, the plant expresses a nucleic acid construct which may comprise a mutant SP1 nucleic acid, for example a mutant of SEQ ID NO. 1 or 2, which encodes a mutant SP1 peptide which carries a mutation, for example in the IMS domain (which controls target recognition) which renders the peptide non-functional wherein said mutation is not a mutation, for example a point mutation, as described in reference 31.

In another aspect, the invention relates to a product derived from a plant as described above, for example a food or feed composition.

As described above, in a second aspect, the invention relates to a method for altering plastid development in a transgenic plant cell, plant or a part thereof which may comprise
a) altering the expression of a nucleic acid which may comprise SEQ ID NO. 1 or 2 and encoding a SP1 peptide which may comprise SEQ ID NO. 3, a functional homolog or variant thereof or
b) altering the activity of a SP1 peptide which may comprise SEQ ID NO. 3 which may comprise expressing in a plant a nucleic acid which may comprise a mutant SEQ ID NO. 1 or 2 and encoding a mutant SP1 peptide, a functional homolog or variant thereof which carries a mutation in the RING domain.

The plastid may be selected from etioplasts, chloroplasts, amyloplasts, elaioplasts, chromoplasts or gerontoplasts. In one embodiment, the level of expression is increased. For example, the overall level of SP1 gene expression is increased or the level of SP1 gene expression in a particular plant organ or cell type is increased. In one embodiment, the method may comprise introducing and expressing in a plant a nucleic acid construct which may comprise SEQ ID NO. 1 or 2 and encoding a SP1 peptide which may comprise SEQ ID NO. 3, a functional homolog or variant thereof. Preferably, plastid development is accelerated. As described above, the construct can further may comprise a regulatory sequence, for example a tissue-specific, such as a seedling-specific, or a strong or constitutive promoter. In one embodiment, the invention relates to a method for accelerating the transition from etioplasts to chloroplasts in seedlings which may comprise introducing and expressing in a plant a nucleic acid construct which may comprise SEQ ID NO. 1 and encoding a SP1 peptide which may comprise SEQ ID NO. 3, a functional homolog or variant thereof. In one embodiment, the invention relates to a method for accelerating the transition from chloroplasts to chromoplasts in fruit which may comprise introducing and expressing in a plant a nucleic acid construct which may comprise SEQ ID NO. 1 and encoding a SP1 peptide which may comprise SEQ ID NO. 3, a functional homolog or variant thereof. In one embodiment, the invention relates to a method for accelerating the transition from proplastids to amyloplasts which may comprise introducing and expressing in a plant a nucleic acid construct which may comprise SEQ ID NO. 1 and encoding a SP1 peptide which may comprise SEQ ID NO. 3, a functional homolog or variant thereof.

The invention also relates to an embodiment of the method for altering plastid development wherein said method may comprise inactivating, repressing or down-regulating the expression of a nucleic acid which may comprise SEQ ID NO. 1 or 2 and encoding a SP1 peptide which may comprise SEQ ID NO. 3, a functional homolog or variant thereof. Preferably, plastid development is delayed. As described above, according to this embodiment, expression of the endogenous SP1 nucleic acid which may comprise SEQ ID NO. 1 or 2 and encoding a SP1 peptide which may comprise SEQ ID NO. 3, a functional homolog or variant thereof can be silenced. Thus, in one embodiment of the invention, the method may comprise introducing and expressing in said plant cell, plant or part thereof a nucleic acid construct which may comprise a RNAi, snRNA, dsRNA, siRNA, miRNA, ta-siRNA, amiRNA or co-suppression molecule that targets the expression of an SP1 gene as described herein.

In a preferred embodiment, the invention relates to a method for delaying the transition from chloroplast to gerontoplast in a plant or plant part, such as a leaf, which may comprise inactivating, repressing or down-regulating the expression of a nucleic acid which may comprise SEQ ID NO. 1 or 2 and encoding a SP1 peptide which may comprise SEQ ID NO. 3, a functional homolog or variant thereof. This delays leaf senescence (‘stay-green’ trait) and thus increases yield.

The invention also relates to an embodiment of the method for altering plastid development wherein said method may comprise altering the activity of a SP1 peptide which may comprise SEQ ID NO. 3 which may comprise expressing in a plant a mutant SEQ ID NO. 1 or 2 nucleic acid which encodes a mutant SP1 peptide, a functional homolog or variant thereof which carries a mutation in the RING domain or which carries a mutation in the IMS domain (which controls target recognition) wherein said mutation is not a point mutation as described in reference 31. The activity is inactivated, repressed or decreased.

In one embodiment, this method may comprise introducing and expressing in a plant a nucleic acid construct which may comprise a mutant SEQ ID NO. 1 or 2 and encoding a mutant SP1 peptide, a functional homolog or variant thereof which carries a mutation in the RING domain.

In other words, a SP1 transgene that encodes a mutant peptide is introduced into the plant using transformation methods known in the art. Stable transformants that express the transgene are generated. The nucleic acid construct preferably may comprise regulatory sequences. This is described elsewhere and can be a promoter that directs overexpression of SP1 or expression of SP1 in a specific tissue, for example in leaves.

The mutation may be a substitution, deletion of one or more residue within the RING domain or an insertion into the RING domain. As described above, the RING is preferably a class C3HC4/HCa RING domain with the consensus sequence:

(SEQ ID NO: 59)
C-X₁₋₂-C-X_{10-16,} _{20/28}-C-X₁₋₂X-H-X₁₋₂-X-C-X₂-C-X₃-X-X₆₋₃₀-
C-P-X-C-Xₙ

Wherein n designates 0-20 amino acids

The RNF domain has highly conserved C and H residues which are metal ligands. These are found at positions 1, 4, 16, 22, 24, 32 and 35 in AtSP1 (using the numbering of RING residues in SEQ ID No. 4). In one embodiment, one or more of these residues in ATSP1 or at one or more corresponding position in a ATSP1 homolog is deleted or substituted. The substitution may be with A. A preferred mutation is at position C330, for example C330A, in AtSP1 and or at a C at a corresponding position in a functional homologue. A shown in the examples, this leads to loss of function of the protein.

In one embodiment, the methods of the invention may comprise comparing the activity of the SP1 polypeptide and/or expression of the SP1 gene with the activity of the SP1 polypeptide and/or expression of the SP1 gene in a control plant.

The methods of the invention may also optionally comprise the steps of screening and selecting plants for those that may comprise a polynucleotide construct as above, have altered expression of the SP1 nucleic acid or altered activity/presence of the SP1 peptide compared to a control plant. Preferably, according to the methods described herein, the progeny plant is stably transformed and may comprise the exogenous polynucleotide which is heritable as a fragment of DNA maintained in the plant cell and the method may include steps to verify that the construct is stably integrated. The method may also comprise the additional step of collecting seeds from the selected progeny plant. A further step can include assessing and/or measuring plastid development, seedling development (for example by measuring seedling survival, emergence or growth), leaf senescence (for example by measuring chlorophyll content) or fruit development. In one embodiment, germplasm is screened.

The invention also relates to a method for delaying green tissue, for example, leaf senescence (‘stay-green’) which may comprise inactivating, repressing or down-regulating the expression of a nucleic acid which may comprise SEQ ID NO. 1 and encoding a SP1 peptide which may comprise SEQ ID NO. 3, a functional homolog or variant thereof.

The invention also relates to a method for increasing yield of a plant by either increasing or inactivating, repressing or down-regulating the expression of a nucleic acid which may comprise SEQ ID NO. 1 or 2 and encoding a SP1 peptide which may comprise SEQ ID NO. 3, a functional homolog or variant thereof. This delays senescence of green tissue and hence increases yield.

In one embodiment, invention also relates to a method for increasing yield of a plant by increasing expression of SP1 and/or activity of SP1 which may comprise a method of introducing and expressing in a plant a nucleic acid construct which may comprise SEQ ID NO. 1 or 2 and encoding a SP1 peptide which may comprise SEQ ID NO. 3, a functional homolog or variant thereof. This accelerates amyloplast development and therefore increases grain size and starch content. This increases yield.

Preferably, a green tissue specific promoter is used. In one embodiment, a strong or constitutive promoter is used. In one embodiment, yield is increased under stress conditions, including moderate or severe stress. The stress may be biotic or abiotic stress, including elevated temperature, cold, drought, salinity, oxidative or osmotic stress or pathogen invasion. In another embodiment, the invention also relates to a method for increasing yield of a plant which may comprise introducing and expressing in a plant a nucleic acid construct which may comprise SEQ ID NO. 1 or 2 and encoding a SP1 peptide which may comprise SEQ ID NO. 3, a functional homolog or variant thereof wherein said promoter is preferably a seed or seedling specific promoter. This leads to accelerated transition of proplastids to amyloplasts and thus increased seed/grain size (a yield-related train).

The invention also relates to a method for increasing seedling emergence, growth and survival which may comprise introducing and expressing in a plant a nucleic acid construct which may comprise SEQ ID NO. 1 or 2 and encoding a SP1 peptide which may comprise SEQ ID NO. 3, a functional homolog or variant thereof. In one embodiment, a seed or seedling specific promoter is used.

The invention also relates to a method for increasing grain size which may comprise introducing and expressing in a plant a nucleic acid construct which may comprise SEQ ID NO. 1 or 2 and encoding a SP1 peptide which may comprise SEQ ID NO. 3, a functional homolog or variant thereof. In one embodiment, a seed or seed specific promoter is used.

The invention also relates to a method for accelerating/delaying fruit ripening which may comprise
a) altering the expression of a nucleic acid which may comprise SEQ ID NO. 1 or 2 and encoding a SP1 peptide which may comprise SEQ ID NO. 3, a functional homolog or variant thereof or
b) altering the activity of a SP1 peptide comprising SEQ ID NO. 3 which may comprise expressing in a plant a nucleic acid which may comprise a mutant SEQ ID NO. 1 or 2 and encoding a mutant SP1 peptide, a functional homolog or variant thereof which carries a mutation in the RING domain.

In one embodiment, fruit ripening is accelerated by increasing expression of a nucleic acid which may comprise SEQ ID NO. 1 or 2 and encoding a SP1 peptide which may comprise SEQ ID NO. 3, a functional homolog or variant thereof. To this end, the plant expresses a nucleic acid construct which may comprise a nucleic acid comprising SEQ ID NO. 1 or 2 and encoding a SP1 peptide which may comprise SEQ ID NO. 3, a functional homolog or variant thereof.

In one embodiment, fruit ripening is delayed by inactivating, repressing or down-regulating the expression of a nucleic acid which may comprise SEQ ID NO 1 or 2 and encoding a SP1 peptide which may comprise SEQ ID NO. 3, a functional homolog or variant thereof.

In these methods, as explained above, expression is increased by expressing a nucleic acid construct which may comprise a nucleic acid which may comprise SEQ ID NO. 1 or 2 and encoding a SP1 peptide which may comprise SEQ ID NO. 3, a functional homolog or variant thereof in said plant or expression is decreased by expressing a nucleic acid construct that targets a nucleic acid which may comprise SEQ ID NO. 1 or 2 and encoding a SP1 peptide which may comprise SEQ ID NO. 3, a functional homolog or variant thereof and reduces expression of the SP1 gene.

The terms “increase”, “improve” or “enhance” are interchangeably used herein. Yield or SP1 expression levels for example are increased by at least a 3%, 4%, 5%, 6%, 7%, 8%, 9% or 10%, preferably at least 15% or 20%, more preferably 25%, 30%, 35%, 40% or 50% or more in comparison to a control plant. SP1 expression levels can be measured by routine methods in the art and compared to control plants.

The terms “reduce” or “decrease” used herein are interchangeable. A decrease, for example in SP1 expression may be 3%, 4%, 5%, 6%, 7%, 8%, 9% or 10%, preferably at least 15% or 20%, more preferably 25%, 30%, 35%, 40% or 50% or more in comparison to a control plant.

Transformation methods are known in the art. Thus, according to the various aspects of the invention, a nucleic acid which may comprise a SP1 nucleic acid, for example SEQ D No. 1, 2, a functional variant or homolog thereof is introduced into a plant and expressed as a transgene. The nucleic acid sequence is introduced into said plant through a process called transformation. The term “introduction” or “transformation” as referred to herein encompasses the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated there from. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell may then be used to regenerate a transformed plant in a manner known to persons skilled in the art.

The transfer of foreign genes into the genome of a plant is called transformation. Transformation of plants is now a routine technique in many species. Advantageously, any of several transformation methods may be used to introduce the gene of interest into a suitable ancestor cell. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts, electroporation of protoplasts, microinjection into plant material, DNA or RNA-coated particle bombardment, infection with (non-integrative) viruses and the like. Transgenic plants, including transgenic crop plants, are preferably produced via Agrobacterium tumefaciens mediated transformation.

To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility is growing the seeds, if appropriate after sterilization, on agar plates using a suitable selection agent so that only the transformed seeds can grow into plants. Alternatively, the transformed plants are screened for the presence of a selectable marker such as the ones described above.

Following DNA transfer and regeneration, putatively transformed plants may also be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

In another aspect, the invention relates to an isolated nucleic acid sequence which may comprise or may consist of SEQ ID NO. 2. In another aspect, the invention relates to a vector which may comprise a SP1 nucleic acid. The SP1 nucleic acid may comprise SEQ D No. 1, 2, 7, 8, 10 or 11 a functional variant or homolog thereof. Homologs of SP1 are defined elsewhere herein. The invention relates to an isolated nucleic acid sequence which may comprise or may consist of SEQ ID NO. 8 or 11. In another aspect, the invention relates to a vector which may comprise a SP1 nucleic acid encoding a peptide as identified in SEQ ID NO. 9, 31, 32, 33, 34, 35, 36, 37, 38, 39, 40, 41, 42, 43, 44, 45, 46, 47, 48, 49, 50, 51, 52, 53, 54, 55 or 56.

Preferably, the vector further may comprise a regulatory sequence which directs expression of the nucleic acid.

The invention also relates to an isolated host cell transformed with a nucleic acid or vector as described above. The host cell may be a bacterial cell, such as Agrobacterium tumeaciens, or an isolated plant cell. The invention also relates to a culture medium or kit which may comprise a culture medium and an isolated host cell as described above.

The nucleic acid or vector described above is used to generate transgenic plants using transformation methods known in the art.

Thus, the methods, vectors and plants of the invention encompass isolated SP1 homologues that modulate plastid development and which hybridize under stringent conditions to the AtSP1 or AtSP1 homologues described herein, or to fragments thereof.

For example, according to the various aspects of the invention, a nucleic acid encoding SP1 may be expressed in said plant by recombinant methods: In another embodiment, SP1 from a given plant may be expressed in any plant of another species as defined herein by recombinant methods, for example AtSP1 may be expressed in a monocot plant.

In a further aspect, the invention relates to a method for making a transgenic plant with altered plastid development which may comprise
a) introducing and expressing in said plant a nucleic acid construct which may comprise SEQ ID NO. 1 or 2 and encoding a SP1 peptide which may comprise SEQ ID NO. 3, a functional homolog or variant thereof or which may comprise a mutant SEQ ID NO. 1 or 2 which has a mutation in the RING domain or which carries a mutation in the IMS domain wherein said mutation is not a as described in reference 31.
b) introducing and expressing in said plant a nucleic acid construct that down-regulates expression of a nucleic acid which may comprise SEQ ID NO. 1 or 2.

Said constructs may comprise a regulatory sequence as described herein.

The invention also relates to the use of a nucleic acid which may comprise SEQ ID NO. 1 or 2 and encoding a SP1 peptide which may comprise SEQ ID NO. 3, a functional homolog or variant thereof or a nucleic acid encoding a mutant SP1 protein in altering plastid development. The functional homologue may, for example, encode a peptide as define din SEQ ID No. 9, 31, 32, 33, 34, 35, 36, 37, 38, 39, 40, 41, 42, 43, 44, 45, 46, 47, 48, 49, 50, 51, 52, 53, 54, 55 or 56. Plastid development may be accelerated, for example in seedlings or fruit or delayed, for example in green tissue such as leaves or fruit. In one embodiment, the invention relates to the use of a nucleic acid which may comprise SEQ ID NO. 1 or 2 and encoding a SP1 peptide which may comprise SEQ ID NO. 3 a functional homolog or variant thereof in accelerating the transition from etioplasts to chloroplasts or the transition from proplastids to amyloplasts. The invention also relates to the use of a siRNA which targets a nucleic acid which may comprise SEQ ID NO. 1 or 2 and encoding a SP1 peptide which may comprise SEQ ID NO. 3 a functional homolog or variant thereof in altering plastid development. The functional homologue may, for example, encode a peptide as define din SEQ ID No. 9, 31, 32, 33, 34, 35, 36, 37, 38, 39, 40, 41, 42, 43, 44, 45, 46, 47, 48, 49, 50, 51, 52, 53, 54, 55 or 56. In one embodiment, the transition of chloroplasts to gerontoplast is delayed. In one embodiment, the transition of chloroplasts to chromoplasts is delayed. Thus, the invention relates to the use of a nucleic acid which may comprise SEQ ID NO: 1 or 2 and encoding a SP1 peptide which may comprise SEQ ID NO. 3 a functional homolog or variant thereof or a siRNA or amiRNA which targets a nucleic acid which may comprise SEQ ID NO. 1 or 2 and encoding a SP1 peptide which may comprise SEQ ID NO. 3 a functional homolog or variant thereof in increasing yield-related traits.

In yet a further aspect, the invention relates to a method for producing a mutant plant expressing a SP1 variant and which is characterised by one of the phenotypes described herein wherein said method uses mutagenesis and Targeting Induced Local Lesions in Genomes (TILLING) to target the gene expressing a SP1 polypeptide. The method may comprise mutagenising a plant population and selecting a plant with altered plastid development and identifying the SP1 variant. For example, mutagenesis is carried out using TILLING where traditional chemical mutagenesis is flowed by high-throughput screening for point mutations. The plants are screened for one of the phenotypes described herein, for example a plant that shows delayed/accelerated plastid development or improved yield. A SP1 locus is then analysed to identify a specific SP1 mutation responsible for the phenotype observed. Plants can be bred to obtain stable lines with the desired phenotype and carrying a mutation in a SP1 locus. In one embodiment, germplasm is screened.

Thus, plants with different genotypes, induced through artificial means, or alternatively having originated through natural sequence divergence, may be screened for the expression of the endogenous SP1 gene to identify germplasm or plants with particular plastid development or yield characteristics.

In one embodiment, the method used to create and analyse mutations is targeting induced local lesions in genomes. In this method, seeds are mutagenised with a chemical mutagen. The mutagen may be fast neutron irradiation or a chemical mutagen, for example selected from the following non-limiting list: ethyl methanesulfonate (EMS), methylmethane sulfonate (MMS), N-ethyl-N-nitrosurea (ENU), triethylmelamine (1′EM), N-methyl-N-nitrosourea (MNU), procarbazine, chlorambucil, cyclophosphamide, diethyl sulfate, acrylamide monomer, melphalan, nitrogen mustard, vincristine, dimethylnitosamine, N-methyl-N′-nitro-nitrosoguanidine (MNNG), nitrosoguanidine, 2-aminopurine, 7,12 dimethyl-benz(a)anthracene (DMBA), ethylene oxide, hexamethylphosphoramide, bisulfan, diepoxyalkanes (diepoxyoctane (DEO), diepoxybutane (BEB), and the like), 2-methoxy-6-chloro-9 [3-(ethyl-2-chloroethyl)aminopropylamino]acridine dihydrochloride (ICR-170) or formaldehyde.

The resulting M1 plants are self-fertilised and the M2 generation of individuals is used to prepare DNA samples for mutational screening. DNA samples are pooled and arrayed on microtiter plates and subjected to gene specific PCR. The PCR amplification products may be screened for mutations in the SP1 target gene using any method that identifies heteroduplexes between wild-type and mutant genes. For example, but not limited to, denaturing high pressure liquid chromatography (dHPLC), constant denaturant capillary electrophoresis (CDCE), temperature gradient capillary electrophoresis (TGCE), or by fragmentation using chemical cleavage. Preferably the PCR amplification products are incubated with an endonuclease that preferentially cleaves mismatches in heteroduplexes between wild-type and mutant sequences. Cleavage products are electrophoresed using an automated sequencing gel apparatus, and gel images are analyzed with the aid of a standard commercial image-processing program. Any primer specific to the SP1 gene may be utilized to amplify the SP1 genes within the pooled DNA sample. Preferably, the primer is designed to amplify the regions of the SP1 gene where useful mutations are most likely to arise, specifically in the areas of the SP1 gene that are highly conserved and/or confer activity. To facilitate detection of PCR products on a gel, the PCR primer may be labelled using any conventional labelling method.

Rapid high-throughput screening procedures thus allow the analysis of amplification products for identifying a mutation conferring the reduction or inactivation of the expression of the SP1 gene as compared to a corresponding non-mutagenised wild-type plant. Once a mutation is identified in a gene of interest, the seeds of the M2 plant carrying that mutation are grown into adult M3 plants and screened for the phenotypic characteristics associated with the SP1 gene. Loss of and reduced function mutants with increased yield or increased/delayed plastid development compared to a control plant can thus be identified.

Plants obtained or obtainable by such method which carry a functional mutation in the endogenous SP1 locus are also within the scope of the invention. The mutation may increase or decrease activity of the mutant SP1 peptide. Thus, in another aspect, the invention relates to a plant cell, plant or a part thereof characterised in that wherein said plant expresses a nucleic acid which may comprise a mutant SEQ ID NO. 1 or 2 and encoding a mutant SP1 peptide, a functional homologue or variant thereof which carries a mutation and wherein said mutation is not a point mutation in the IMS domain as disclosed in reference 31. The mutation is preferably in the RING domain.

The various aspects of the invention described herein clearly extend to any plant cell or any plant produced, obtained or obtainable by any of the methods described herein, and to all plant parts and propagules thereof unless otherwise specified. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced by the parent in the methods according to the invention.

The invention also extends to harvestable parts of a plant of the invention as described above such as, but not limited to seeds, leaves, fruits, flowers, stems, roots, rhizomes, tubers and bulbs. The invention furthermore relates to products derived, preferably directly derived, from a harvestable part of such a plant, such as dry pellets or powders, oil, fat and fatty acids, starch or proteins. The invention also relates to food products and food supplements which may comprise the plant of the invention or parts thereof.

The invention also relates to a method for modifying the activity of a TOC protein in a plant activity which may comprise
a) altering the expression of a nucleic acid which may comprise SEQ ID NO. 1 or 2 and encoding a SP1 peptide which may comprise SEQ ID NO. 3, a functional homolog or variant thereof or
b) altering the activity of a SP1 peptide which may comprise SEQ ID NO. 3 which may comprise expressing in a plant a nucleic acid which may comprise a mutant SEQ ID NO. 1 or 2 and encoding a mutant SP1 peptide, a functional homolog or variant thereof which carries a mutation in the RING domain.

The invention also relates to accelerating/delaying chloroplast development
a) altering the expression of a nucleic acid which may comprise SEQ ID NO. 1 or 2 and encoding a SP1 peptide which may comprise SEQ ID NO. 3, a functional homolog or variant thereof or
b) altering the activity of a SP1 peptide which may comprise SEQ ID NO. 3 which may comprise expressing in a plant a nucleic acid which may comprise a mutant SEQ ID NO. 1 or 2 and encoding a mutant SP1 peptide, a functional homolog or variant thereof which carries a mutation in the RING domain.

In one embodiment, chloroplast development is accelerated by increasing expression of a nucleic acid which may comprise SEQ ID NO. 1 or 2 and encoding a SP1 peptide which may comprise SEQ ID NO. 3, a functional homolog or variant thereof. To this end, the plant expressing a nucleic acid construct which may comprise a nucleic acid which may comprise SEQ ID NO. 1 or 2 and encoding a SP1 peptide which may comprise SEQ ID NO. 3, a functional homolog or variant thereof. In one embodiment, chloroplast development is delayed by inactivating, repressing or down-regulating the expression of a nucleic acid which may comprise SEQ ID NO. 1 or 2 and encoding a SP1 peptide which may comprise SEQ ID NO. 3, a functional homolog or variant thereof.

The inventor has also shown that SP1 is involved in regulating stress response. The examples demonstrate that SP1 acts to down-regulate the TOC machinery under stress conditions, reducing import of photosynthetic apparatus components and thereby reducing the tendency for ROS generation and redox damage. Thus, increasing the expression of SP1 leads to an increased tolerance to stress compared to a control plant. The terms tolerance and resistance are used interchangeably herein.

In another aspect, the invention therefore relates to a method for increasing stress tolerance to abiotic stress, preferably to one or more of salinity, osmotic stress and/or oxidative stress in a plant cell, plant or part thereof which may comprise introducing and expressing in said plant cell, plant or part thereof a nucleic acid construct which may comprise a nucleic acid which may comprise SEQ ID NO. 1 or 2 and encoding a SP1 peptide which may comprise SEQ ID NO. 3, a functional homologue or variant thereof.

The terms functional homologue or variant and plant are all explained elsewhere herein and preferred embodiments that are also applicable to this aspect of the invention are given elsewhere herein.

The nucleic acid construct may comprise a regulatory sequence as defined elsewhere herein, for example a stress-inducible, constitutive or strong promoter.

The stress is one or more of salinity, osmotic stress and/or oxidative stress. In one embodiment, the stress is salinity. In one embodiment, the stress is osmotic stress. In one embodiment, the stress is oxidative stress.

The tolerance of plants to different types of abiotic stresses is not necessarily conferred through related mechanisms and indeed occurs via different signal transduction pathways. Thus, it cannot be expected that a gene that confers, when expressed, one type of stress, could also confer a different type of stress. Notably and unexpectedly, the inventor has demonstrated herein that SP1 acts to enhance tolerance responses in plants to all three of these different stress factors. The inventor has found that the plants of the invention are more tolerant to more than one stress. Accordingly, in one embodiment, the stress is salinity and osmotic stress. In one embodiment, the stress is salinity and oxidative stress. In one embodiment, the stress is osmotic stress and oxidative stress. In one embodiment, the stress is all of salinity, osmotic stress and oxidative stress.

Salt (salinity) stress can thus refer to moderate or severe salt stress and is present when the soil is saline. Soils are generally classified as saline when the ECe is 4 dS/m or more, which is equivalent to approximately 40 mM NaCl and generates an osmotic pressure of approximately 0.2 MPa. Most plants can however tolerate and survive about 4 to 8 dS/m although this will impact on plant fitness and thus yield. For example in rice, soil salinity beyond ECe ˜4 dS/m is considered moderate salinity while more than 8 dS/m becomes high. Similarly, pH 8.8-9.2 is considered as non-stress while 9.3-9.7 as moderate stress and equal or greater than 9.8 as severe stress. Thus, salt stress as used herein refers to an ECe of 4 dS/m or more, for example about 4 to about 8 dS/m or about 40 mM NaCl or more, for example about 40 mM NaCl to about 100 mM NaCl or about 40 mM NaCl to 200 mM NaCl. Exposure to high levels of NaCl not only affects plant water relations but allo creates ionic stress in the form of cellular accumulation of Cl⁻ and, in particular, Na⁺ ions. Salt stress also changes the homeostasis of other ions such as Ca²⁺, K⁺, and NO₃⁻ levels (water deficit, ion toxicity, nutrient imbalance, and oxidative stress), and at least two main responses can be expected: a rapid protective response together with a long term adaptation response. During initial exposure to salinity, plants experience water stress, which in turn reduces leaf expansion. During long-term exposure to salinity, plants experience ionic stress, which can lead to premature senescence of adult leaves, and thus a reduction in the photosynthetic area available to support continued growth. Thus, by increasing tolerance to salt stress, plant yield is increased.

When plant cells are under environmental stress, several chemically distinct reactive oxygen species (ROS) are generated by partial reduction of molecular oxygen and these can cause oxidative stress damage or act as signals. Oxidative stress can be induced by various environmental and biological factors such as hyperoxia, light, drought, high salinity, cold, metal ions, pollutants, xenobiotics, toxins, reoxygenation after anoxia, experimental manipulations, pathogen infection and aging of plant organs.

Auto-oxidation of components of the photosynthetic electron transport chain leads to the formation of superoxide radicals and their derivatives, hydrogen peroxide and hydroxyl radicals. These compounds react with a wide variety of biomolecules including DNA, causing cell stasis and death. Thus, by increasing tolerance to oxidative stress, plant yield is increased.

Thus, the invention relates in particular to methods for increasing or enhancing plant response to oxidative stress, caused for example by extreme temperatures, drought UV light, irradiation, high salinity, cold, metal ions, pollutants, toxins, or pathogen infection by bacteria, viruses or fungi or a combination thereof.

Osmotic adjustment plays a fundamental role in water stress responses and growth in plants. Drought, salinity and freeze-induced dehydration constitute direct osmotic stresses; chilling and hypoxia can indirectly cause osmotic stress via effects on water

uptake and loss. Thus, by increasing tolerance to osmotic stress, plant yield is increased. Osmotic stress in accordance with the invention refers to osmotic stress caused by salinity, freezing or chilling and drought.

According to the different aspects of the invention, plant stress responses are increased, enhanced or improved. This is understood to mean an increase compared to the level as found in a control, for example a wild-type plant. A skilled person will appreciate that such stress responses can be measured and the increase can be 2- to 10-fold.

The methods of the invention may also optionally comprise the steps of screening and selecting plants for those that may comprise a polynucleotide construct as above, have increased stress resistance to one or more of salinity, osmotic stress and/or oxidative stress compared to a control plant. Preferably, according to the methods described herein, the progeny plant is stably transformed and may comprise the exogenous polynucleotide which is heritable as a fragment of DNA maintained in the plant cell and the method may include steps to verify that the construct is stably integrated. The method may also comprise the additional step of collecting seeds from the selected progeny plant. A further step can include exposing the plants to severe, moderate or mild stress and assessing and/or measuring salinity, osmotic stress and/or oxidative stress and optionally comparing this to a control plant. A further step can include measuring yield and optionally comparing yield to a control plant.

The invention also relates to a method for reducing the amount of ROS in a plant in response to stress which may comprise introducing and expressing in said plant cell, plant or part thereof a nucleic acid construct which may comprise a nucleic acid which may comprise SEQ ID NO. 1 or 2 and encoding a SP1 peptide which may comprise SEQ ID NO. 3, a functional homologue or variant thereof.

The invention also relates to a method for increasing yield under stress conditions which may comprise introducing and expressing in said plant cell, plant or part thereof a nucleic acid construct which may comprise a nucleic acid which may comprise SEQ ID NO. 1 or 2 and encoding a SP1 peptide which may comprise SEQ ID NO. 3, a functional homologue or variant thereof wherein said stress is selected from salinity, osmotic stress and/or oxidative stress.

The invention also relates to the use of a nucleic acid which may comprise SEQ ID NO. 1 or 2 and encoding a SP1 peptide which may comprise SEQ ID NO. 3, a functional homologue or variant thereof in increasing stress tolerance of a plant wherein said stress is selected from salinity, osmotic stress and/or oxidative stress. The functional homologue may, for example, encode a peptide as define din SEQ ID No. 9, 31, 32, 33, 34, 35, 36, 37, 38, 39, 40, 41, 42, 43, 44, 45, 46, 47, 48, 49, 50, 51, 52, 53, 54, 55 or 56.

The invention also relates to a method for producing a stress tolerant plant which may comprise introducing and expressing in said plant cell, plant or part thereof a nucleic acid construct which may comprise a nucleic acid which may comprise SEQ ID NO. 1 or 2 and encoding a SP1 peptide which may comprise SEQ ID NO. 3, a functional homologue or variant thereof wherein said stress is selected from salinity, osmotic stress and/or oxidative stress.

According to the various aspects of the invention, the stress may be severe or preferably moderate stress. In Arabidopsis research, stress is often assessed under severe conditions that are lethal to wild-type plants. For example, drought tolerance is assessed predominantly under quite severe conditions in which plant survival is scored after a prolonged period of soil drying. However, in temperate climates, limited water availability rarely causes plant death, but restricts biomass and seed yield. Moderate water stress, that is suboptimal availability of water for growth can occur during intermittent intervals of days or weeks between irrigation events and may limit leaf growth, light interception, photosynthesis and hence yield potential. Leaf growth inhibition by water stress is particularly undesirable during early establishment. There is a need for methods for making plants with increased yield under moderate stress conditions. In other words, whilst plant research in making stress tolerant plants is often directed at identifying plants that show increased stress tolerance under severe conditions that will lead to death of a wild-type plant, these plants do not perform well under moderate stress conditions and often show growth reduction which leads to unnecessary yield loss:

Thus, in one embodiment of the methods of the invention, yield is improved under moderate stress conditions. The transgenic plants according to the various aspects of the invention show enhanced tolerance to these types of stresses compared to a control plant and are able to mitigate any loss in yield/growth. The tolerance can therefore be measured as an increase in yield as shown in the examples. The terms moderate or mild stress/stress conditions are used interchangeably and refer to non-severe stress. In other words, moderate stress, unlike severe stress, does not lead to plant death. Under moderate, that is non-lethal, stress conditions, wild-type plants are able to survive, but show a decrease in growth and seed production and prolonged moderate stress can also result in developmental arrest. The decrease can be at least 5%-50% or more. Tolerance to severe stress is measured as a percentage of survival, whereas moderate stress does not affect survival, but growth rates. The precise conditions that define moderate stress vary from plant to plant and also between climate zones, but ultimately, these moderate conditions do not cause the plant to die. With regard to high salinity for example, most plants can tolerate and survive about 4 to 8 dS/m. Specifically, in rice, soil salinity beyond ECe ˜4 dS/m is considered moderate salinity while more than 8 dS/m becomes high. Similarly, pH 8.8-9.2 is considered as non-stress while 9.3-9.7 as moderate stress and equal or greater than 9.8 as higher stress.

Thus in one embodiment, the methods of the invention relate to increasing resistance to moderate (non-lethal) stress or severe stress. In the former embodiment, transgenic plants according to the invention show increased resistance to stress and therefore, the plant yield is not or less affected by the stress compared to wild type yields which are reduced upon exposure to stress. In other words, an improve in yield under moderate stress conditions can be observed.

In another aspect, the invention relates to a plant obtainable or obtained by a method as described herein.

While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

All documents mentioned in this specification are incorporated herein by reference in their entirety.

“and/or” where used herein is to be taken as specific disclosure of each of the multiple specified features or components with or without the other at each combination unless otherwise dictated. For example “A, B and/or C” is to be taken as specific disclosure of each of (i) A, (ii) B, (iii) C, (iv) A and B, (v) B and C or (vi) A and B and C, just as if each is set out individually herein.

Unless context dictates otherwise, the descriptions and definitions of the features set out above are not limited to any particular aspect or embodiment of the invention and apply equally to all aspects and embodiments which are described.

The invention is further described in the following non-limiting examples.

### Materials and Methods

### Example 1

### Characterisation of SP1 and its Role in Plastid Development

Plant Growth Conditions and Physiological Studies

With the exception of rpn8a and ppi1 introgressed into Landsberg erecta (Ler), all Arabidopsis thaliana plants were of the Columbia-0 (Col-0) ecotype. The ppi1, tic40-4, hsp93-V-1, toc75-III-3, rpn8a and pbe1 mutants have all been reported previously (7, 12, 25, 31). The sp1-2 (Salk_063571) and sp1-3 (Salk_002099) mutants were obtained from the Salk Institute Genomic Analysis Laboratory, and confirmed by genomic PCR and RT-PCR. For in vitro growth, seeds were surface sterilized, sown on Murashige-Skoog (MS) agar medium in petri plates, cold-treated at 4° C., and thereafter kept in a growth chamber. All plants were grown under a long-day cycle (16 h light, 8 h dark). Chlorophyll measurement was performed using a Konica-Minolta SPAD-502 meter, or following N,N′-dimethylformamide (DMF) extraction using a spectrophotometer (7, 12).

Dark-to-light shift experiments were performed as reported previously (29) with minor modifications. Seeds of identical age were sown on MS medium and cold-treated in the dark for 3 days, and then exposed to light for 6 h to induce germination. Next, the plants were grown in darkness for 5 or 6 days (the longer treatment induced mortality upon illumination and was used to measure survival rates; the shorter treatment facilitated chlorophyll measurements and protein extraction from live plants following illumination). Thereafter, plants were transferred to continuous light for various periods, as indicated, before determining the survival rate (plants with green, expanded cotyledons were scored as survivors), organelle morphology, or chlorophyll content. For survival rate analysis, four experiments were performed and ˜90 seedlings per genotype were analysed in each experiment; for chlorophyll measurements, three experiments were performed and ˜30 seedlings per genotype were analysed in each experiment. Dark treatments for the induction of senescence were conducted as previously described (30). Developmentally-equivalent leaves of 28-day-old plants were wrapped in aluminium foil whilst still attached to the plant, and then left under standard growth conditions for 5 days. Photochemical efficiency of photosystem II (Fv/Fm) was determined by measuring chlorophyll fluorescence as described previously (12). Three experiments were performed, and approximately five leaves (each one from a different plant) were analysed per genotype in each experiment.

Identification of SP1

The sp1 mutant was identified by screening the M2 progeny of 7,000 M1 ppi1 seeds that had been treated with 100 mM ethyl methanesulfonate for 3 h. Mapping of SP1 was conducted by analysing the greenest plants in F2 populations from crosses between sp1-1 ppi1 (Col-0) and ppi1 introgressed into the Ler ecotype, using PCR markers that detect Col-0/Ler polymorphisms. A point mutation was detected at a splice junction of At1g63900, causing mis-splicing of the gene, frame-shifts, and premature termination. This indicated that sp1-1 is a knock-out allele. Transmembrane domains were predicted using Aramemnon (TmMultiCon), and alignments were performed using Clustal W.

Plasmid Constructs, Protoplast Transient Assays, and Generation of Transgenic Lines

All primers used are listed in Table 1. All Arabidopsis coding sequences (CDSs) were PCR-amplified from Col-0 cDNA, and mutants of SP1 were generated by PCR-based in vitro mutagenesis. The YFP CDS was amplified from plant expression vector p2GWY7 which provides a C-terminal YFP tag. The Gateway cloning system (Invitrogen) was used for most SP1-related constructs, and all donor vectors were verified by DNA sequencing. The SP1, SP1-C330A (Cys330-to-Ala) and SPL1 CDSs were cloned into binary overexpression vector pB2GW7, while SP1, SP1ΔTM1/2 (lacking residues 1-20 and 226-243), SPL1 (At1g59560) and SPL2 (At1g54150) were cloned into p2GWY7. The YFP, SP1 and SP1-C330A sequences were cloned into a modified p2GW7 plant expression vector providing a haemagglutinin (HA) epitope-tag at the C-terminus. Sequences encoding atToc33 (At1g02280) and C-terminally FLAG-tagged ubiquitin (AtUBQ10; At4g05320) (26) were PCR-amplified and cloned into p2GW7. Sequences coding for SP1cyt (residues 244-343), SP1cyt-C330A, SP1ims (residues 21-225) and SP1flex (SP1ims and SP1cyt connected with a flexible linker: -[Gly4-Ser]2-) (SEQ ID NO: 60) were sub-cloned into N-terminal GST fusion protein expression vector pDest-565 (Addgene Plasmid Repository) or pGEX-6P-1 (GE Healthcare).

Protoplast isolation and transient assays were carried as known in the art. When required, MG132 (Sigma) was added to the protoplast culture after 15 h incubation to a final concentration of 30 μM, and then the culture was incubated for 2 h more before further analysis. For YFP fluorescence and immunoprecipitation assays, 0.1 ml (105) or 1 ml (106) of protoplasts were transfected with 5 μg or 100 μg of DNA, respectively. The 35S:SP1, 35S:SP1-C330A and 35S:SPL1 transgenic plants (in the sp1 ppi1 or ppi1 backgrounds) were generated by Agrobacterium-mediated transformation using pB2GW7-based vectors (12). At least 12 T2 lines for each combination were analysed and at least two lines with single T-DNA insertion (which showed a 3:1 segregation on phosphinothricin-containing MS medium in the T3 generation) were chosen for further analysis. The 35S:SP1 transgenic plants in the Col-0 and toc75-III-3 backgrounds were obtained by crossing 35S:SP1 ppi1 lines with the corresponding genotypes, followed by PCR testing in the F2 generation.

Microscopy

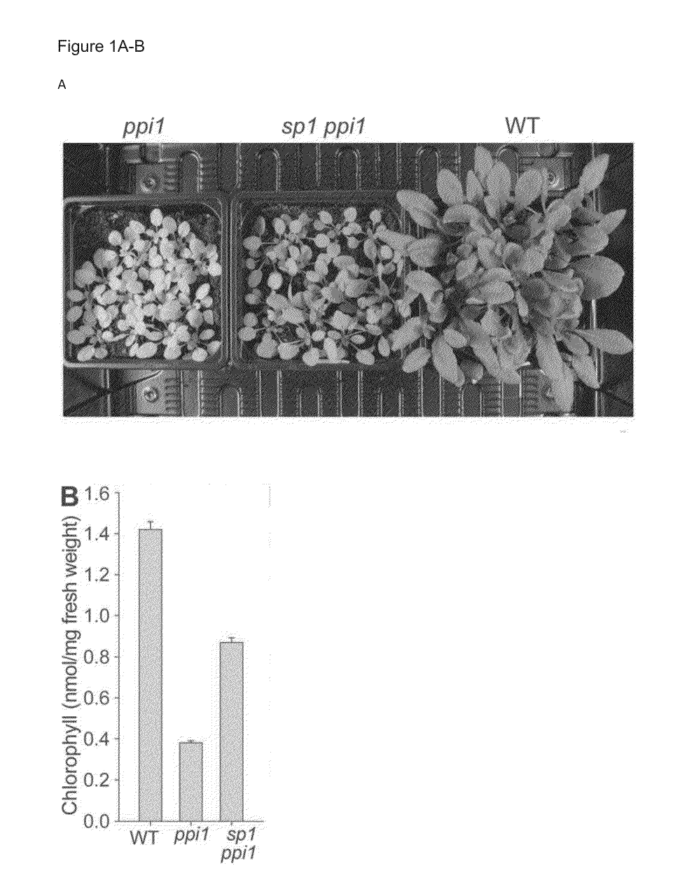

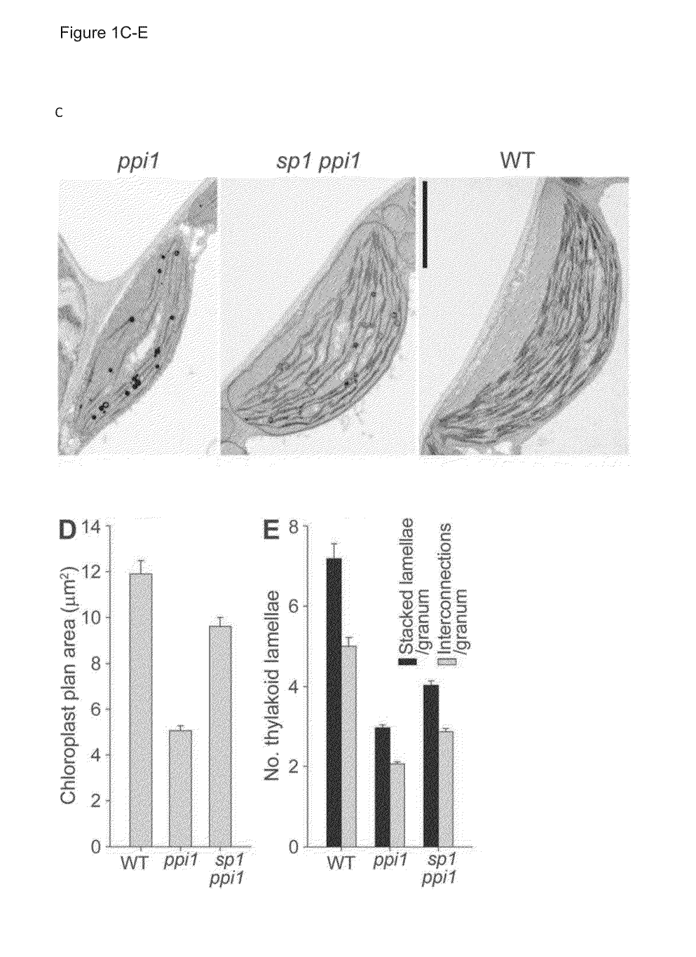

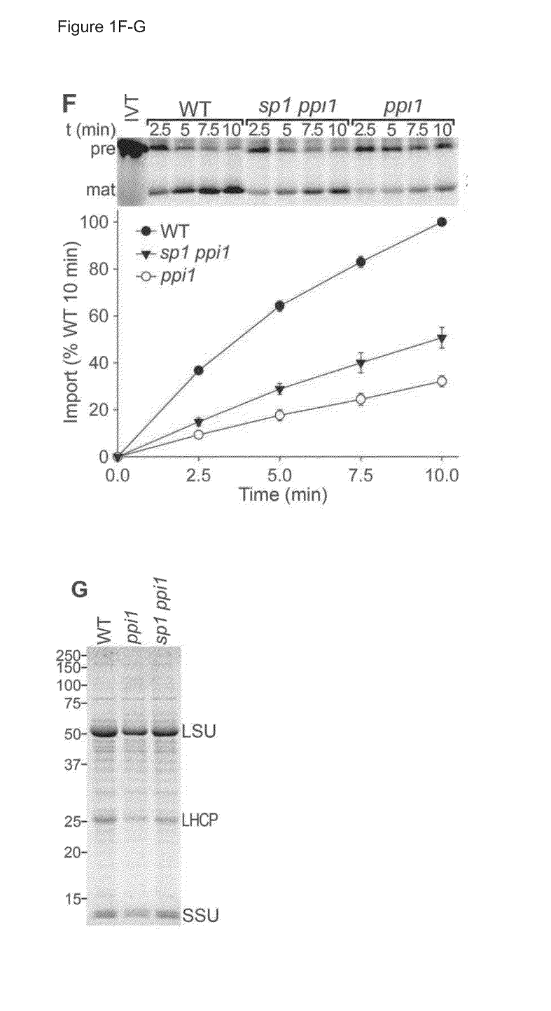

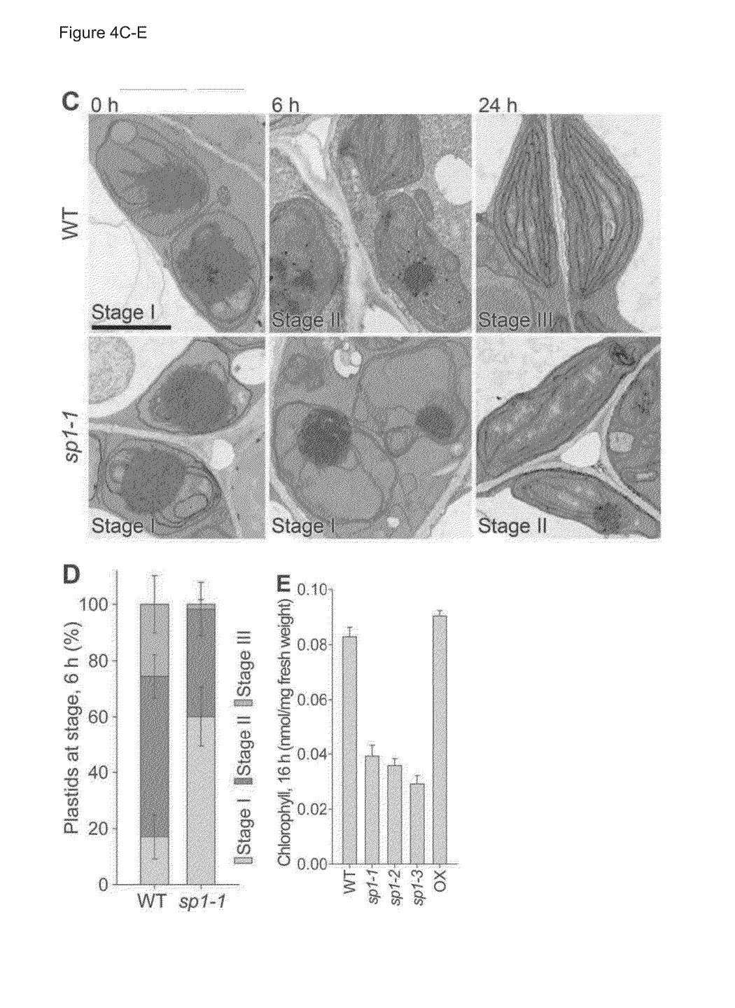

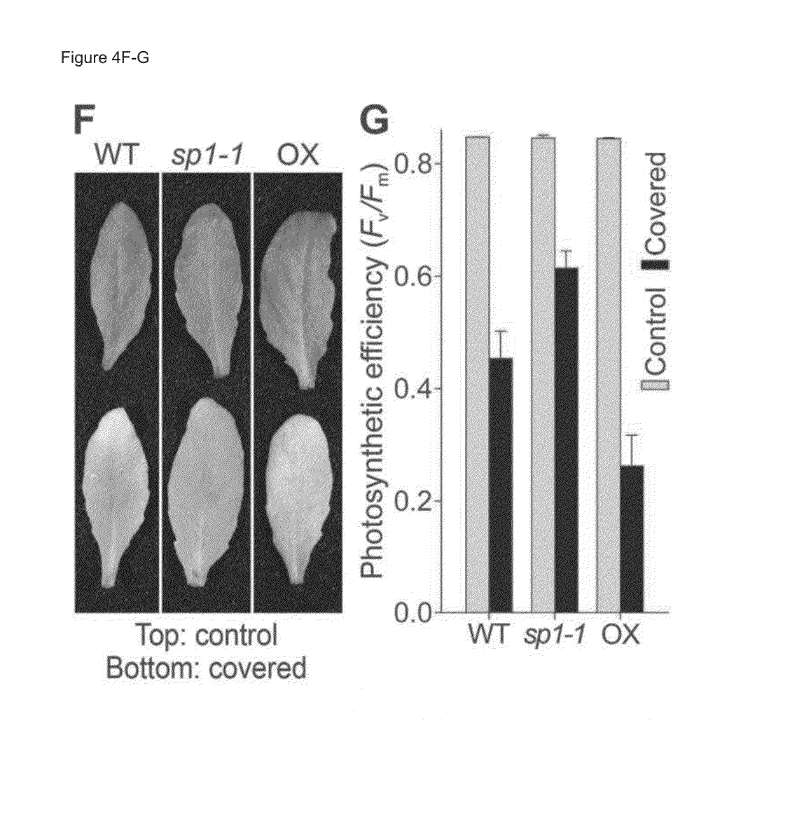

Fluorescence images were captured using a Nikon Eclipse TE-2000E inverted microscope as described previously. All experiments were conducted at least twice with the same results, and typical images are shown. Transmission electron microscopy was performed as described previously (12). Measurements in FIG. 1 (D and E) were taken using at least 30 different plastids per genotype, or about 120 different grana per genotype, and are representative of three individuals per genotype. Chloroplast cross-sectional area was estimated as described (12), using the equation: π×0.25×length×width: For FIG. 4D, a total of ˜180 plastids from nine plants were analysed per genotype. Plastids were assigned to three developmental stages: I, those with large prolamellar bodies (PLBs) and rudimentary prothylakoids; II, those with PLBs of reduced sized and partially developed thylakoids; III, those in which all PLBs had transformed into thylakoids.

Expression and Purification of Recombinant Proteins

For purification of SP1-related proteins with GST tags, plasmids were transformed into Escherichia coli Rosetta (DE3) cells (Novagen), and bacterial cultures were incubated at 37° C. until OD600 reached 0.6-0.8 before induction. Expression of fusion proteins was induced with 0.2 mM isopropyl-β-D-thiogalactopyranoside at 22° C. for 16 h. Cells were lysed by sonication in purification buffer (25 mM Tris-HCl, pH 7.5, 500 mM NaCl, 1% Triton X-100) with a protease inhibitor cocktail (Roche) (free of EDTA for in vitro ubiquitination assays). For purification, glutathione agarose beads (Sigma) were added to cleared lysates and incubated for 4 h at 4° C. with slow rotation, before washing four times with washing buffer (25 mM Tris-HCl, pH 7.5, 300 mM NaCl, 0.1% Triton X-100). For in vitro ubiquitination assays, GST proteins were eluted with 50 mM reduced glutathione in elution buffer (50 mM Tris-HCl, pH 7.5, 150 mM NaCl, 0.1% Triton X-100), and glutathione was then diluted by buffer exchange using Vivaspin 500 ultrafiltration spin columns (Sartorius Stedim Biotech). For in vitro pull-down assays, beads bound with GST proteins were kept in elution buffer with 40% glycerol for direct use or stored at −80° C. The 6×His-tagged (SEQ ID NO: 61) AtUBC8 E2 (At5g41700) was expressed and purified as described previously.

In Vitro Translation and In Vitro Pull-Down

Sequences encoding SP1, SP1-HA-FLAG, atToc33 (At1g02280), atToc33G (residues 1-251), atToc34 (At5g05000), atToc120 (At3g16620), Toc120A (residues 1-343), atToc132 (At2g16640), atToc159 (At4g02510), atToc159A (residues 1-726), atToc159G (residues 727-1091), atToc159M (residues 1092-1503), OEP80 (At5g19620), and SFR2 (At5g19620) were cloned into pBlueScript II SK—using a single SmaI restriction site and verified by DNA sequencing. The atToc75-III (At3g46740) and preSSU (At1g67090) constructs were described previously, as was the in vitro transcription/translation procedure (12). The OEP80 and SFR2 proteins acted membrane protein controls in our assays.

For each combination of the in vitro pull-down assay, 10 μl of translation mixture was pre-cleared by incubation with 10 μl glutathione beads in 250 μl binding buffer (25 mM Tris-HCl, pH 7.5, 5 mM MgCl2, 1 mM dithiothreitol [DTT], 150 mM NaCl, 0.05 mM ZnCl2, 0.1% Triton X-100, 10% glycerol) for 45 min and centrifuged at 14,000 g for 5 min at 4° C. Equal amounts of bead-bound GST proteins blocked with 5% bovine serum albumin in binding buffer were added to the supernatants, and incubated for 2 h at 4° C. with slow rotation. After six washes with 500 μl modified binding buffer (300 mM NaCl instead of 150 mM), the bound proteins were recovered in elution buffer containing 50 mM reduced glutathione. Eluates were mixed with equal volumes of 2×SDS-PAGE loading buffer (50 mM Tris-HCl, pH 6.8, 20% glycerol, 1% sodium dodecyl sulphate [SDS], and 0.1 M DTT) and analysed by SDS-PAGE and phosphorimaging.

Chloroplast Isolation, Import, and Topology Analysis

Chloroplasts were isolated from 14-day-old in vitro grown plants (or, when stated, from protoplasts). Isolations, protein import, and alkaline extraction were performed as described previously (12). Presented import data (FIG. 1F) represent four independent experiments. Protease treatments were performed as described with some minor modifications; 100 μg/ml thermolysin or 500 μg/ml trypsin (with or without 1% Triton X-100) was used unless specified otherwise. After protease treatments, chloroplast pellet was added directly to 2×SDS-PAGE loading buffer, followed by SDS-PAGE and phosphorimaging.

SDS-PAGE, Immunoblotting and Immunoprecipitation

Sodium dodecyl sulphate-polyacrylamide gel electrophoresis (SDS-PAGE) and immunoblotting were performed as described (31, 48) with minor modifications. When necessary, gels were stained with Coomassie Brilliant Blue R250 (Fisher Scientific) or SYPRO Red (Molecular Probes).

Primary antibodies were as follows. To identify TOC proteins or components of the translocon at the inner envelope membrane of chloroplasts (TIC), we employed: anti-atToc75-III POTRA-domain (residues 158-449; antigen for rabbit immunization was bacterially-expressed from pET-23d [Novagen]); anti-atToc159 A-domain (8); anti-atToc132 A-domain (residues 1-431; expressed from pGEX-6P-1 [GE Healthcare]) (new for this study); anti-atToc120 A-domain (residues 1-343; expressed from pGEX-6P-1); anti-atToc33 G-domain (residues 1-262; expressed from pRSET [Invitrogen]); anti-atToc33 peptide antibody (for FIG. S12A); anti-atToc34 C-terminus (residues 170-313; expressed from pQE30 [Qiagen]) (new for this study); anti-atTic110 stromal domain (residues 93-966; expressed from pET-21d); and anti-atTic40 stromal domain (residues 130-447; expressed from pGEX-6P-1). For non-TOC outer envelope proteins, we employed: anti-OEP80 (outer envelope protein, 80 kD); and anti-SFR2 (sensitive to freezing 2). For photosynthetic proteins, we employed: anti-LHCP (light-harvesting chlorophyll a/b-binding protein) (antigen from pea) (12); anti-0E33 (oxygen evolving complex, 33 kD subunit) (antigen from pea) (12); and anti-chloroplast GAPDH (glyceraldehyde-3-phosphate dehydrogenase, subunits GapA and GapB) (antigen from spinach). For non-photosynthetic, housekeeping plastid proteins, we employed: anti-cpHsc70 (chloroplast heat shock cognate protein, 70 kD) (AgriSera, AS08 348); anti-PRPL35 (plastid ribosomal protein L35) (antigen from spinach); CPO (coproporphyrinogen oxidase) (antigen from tobacco) (12). As loading controls, we employed: anti-PMA2 (plasma membrane H+-ATPase 2) (antigen from Nicotiana plumbaginifolia) (12); and anti-H3 histone (Abcam, ab1791). Other primary antibodies we employed were: anti-Hsp93 (heat shock protein, 93 kD) (antigen from pea) (12); anti-HA (haemagglutinin) tag (Sigma, H6908); and anti-FLAG tag (Sigma, A9469).

Secondary antibodies were anti-rabbit IgG conjugated with either alkaline phosphatase (Sigma) or horseradish peroxidase (Santa Cruz Biotechnology), or anti-mouse IgG conjugated with horseradish peroxidase (GE Healthcare) in the case of anti-FLAG. Chemiluminescence was detected using ECL Plus Western Blotting Detection Reagents (GE Healthcare) and an LAS-4000 imager (Fujifilm). Band intensities were quantified using Aida software (Raytest).

For the immunoprecipitation of HA-tagged proteins, total protein (˜500 mg) was extracted from protoplasts in IP buffer (25 mM Tris-HCl, pH 7.5, 150 mM NaCl, 1 mM EDTA, 1% Triton X-100) containing 0.5% plant protease inhibitor cocktail (Sigma), and incubated with protein A-Sepharose CL-4B beads (GE Healthcare) for 15 min before centrifugation at 10,000 g for 10 min at 4° C. to pre-clear. Anti-HA was then added to the supernatant to give a 1:100 dilution and incubated for 2 h at 4° C., followed by incubation with 25 μl protein A-Sepharose beads for 2 h at 4° C. with slow rotation. After six washes with 500 μl IP-washing buffer (25 mM Tris-HCl, pH 7.5, 150 mM NaCl, 1 mM EDTA, 0.5% Triton X-100), bound proteins were eluted by boiling in 2×SDS-PAGE loading buffer for 5 min, and analysed by SDS-PAGE and immunoblotting. A similar procedure was adopted for the immunoprecipitation of FLAG-tagged proteins, except that the pre-clear step was omitted and 50 μl Anti-FLAG M2 Affinity Gel (Sigma) was used instead of primary antibody and protein A-Sepharose beads. When detecting ubiquitinated proteins, the IP buffer also contained 10 mM N-ethylmaleimide (NEM; Sigma).

To assess ubiquitination of SP1 or atToc33, both the target protein and ubiquitin were transiently overexpressed in protoplasts, to increase detection sensitivity for higher molecular Weight forms. Protoplasts were lysed in denaturing buffer (25 mM Tris-HCl, pH 7.5, 150 mM NaCl, 5 mM EDTA, 10 mM NEM, 1% SDS, 2% Sarcosyl, 5 mM DTT), prior to incubation at 75° C. and 600 rpm for 30 min in a Thermomixer Comfort (Eppendorf). The lysate was then diluted by adding 1 volume of 2% Triton X-100 and 8 volumes of IP buffer containing 0.5% plant protease inhibitor cocktail, and incubated on ice for 30 min. Subsequent immunoprecipitation steps were as described above (except that anti-atToc33 was used to precipitate atToc33).

In Vitro Ubiquitination

For the SP1 auto-ubiquitination assay, 1 μg bacterially-expressed GST, GST-SP1 flex, GST-SP1 cyt or GST-SP1 cyt-C330A was incubated with 100 ng human E1 (Merck), 200 ng 6×His-tagged (SEQ ID NO: 61) AtUBC8 E2, and 6 μg HA-ubiquitin (Boston Biochemical) in a 30 μl reaction containing 50 mM Tris-HCl, pH 7.5, 60 mM NaCl, 1 mM DTT, 0.05 mM ZnCl2, and 4 mM MgATP. After incubation at 30° C. for 2 h, reactions were stopped by adding an equal volume of 2×SDS-PAGE loading buffer, and analysed by immunoblotting using anti-HA antibody. To detect ubiquitination of SP1 substrates, 3 μl of rabbit reticulocyte lysate-translated 35S-labeled atToc159, atToc75-III, atToc33 or atToc159G was additionally added in the above reaction system, along with 1 μM ubiquitin aldehyde (Sigma), while reactions were analysed by SDS-PAGE and phosphorimaging. When required, ubiquitin (Sigma) was added instead of HA-ubiquitin.

Results and Discussion

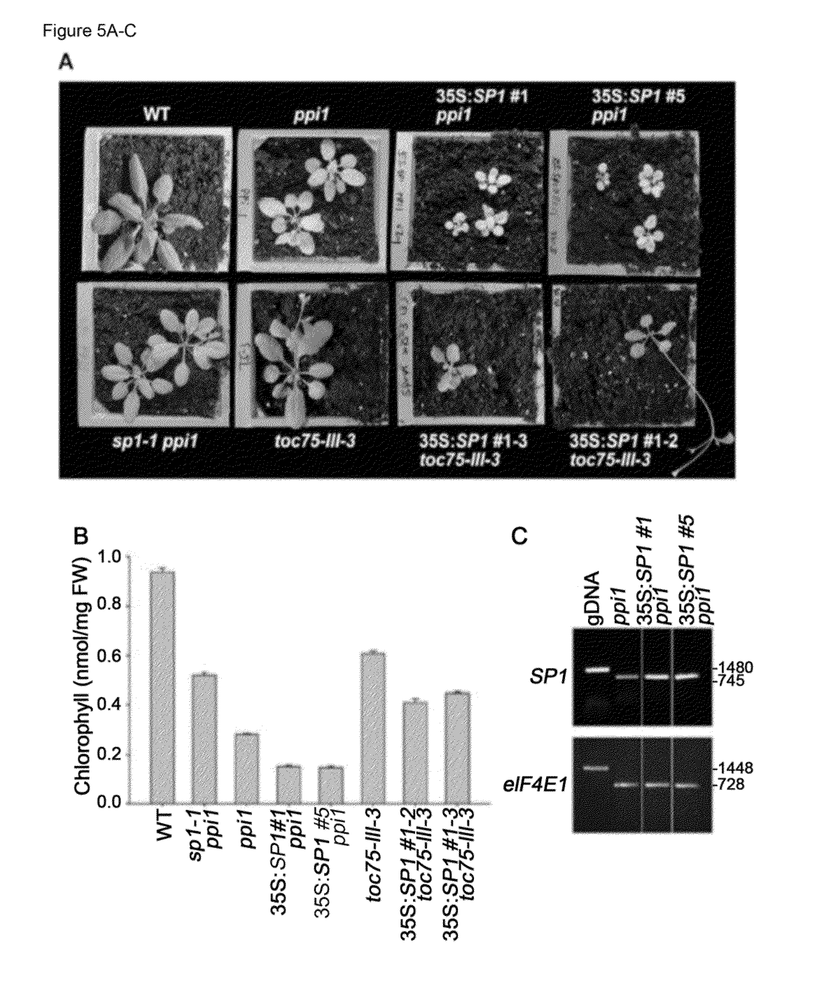

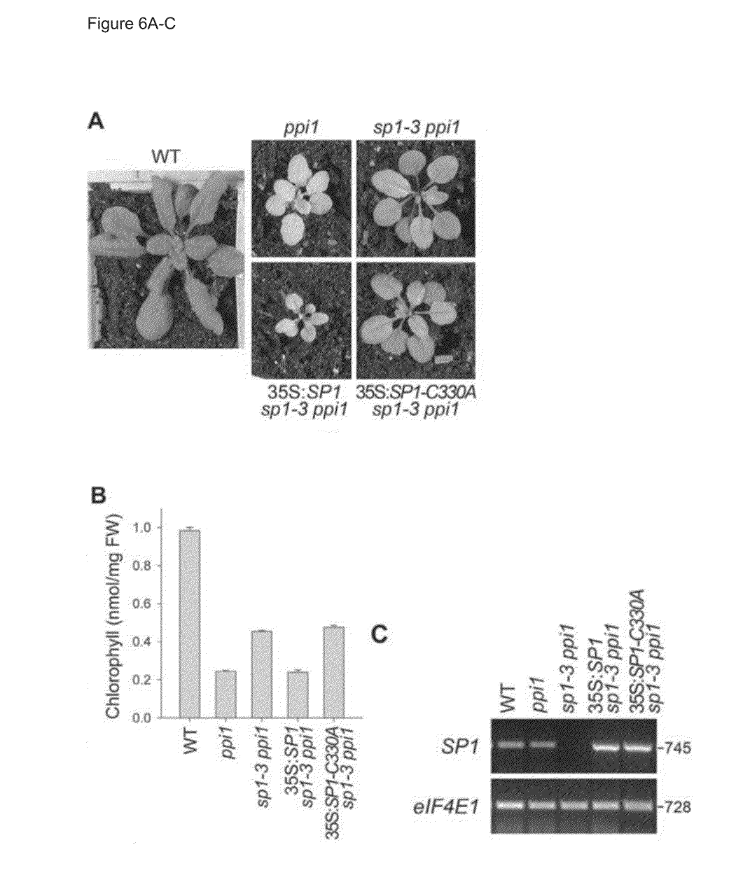

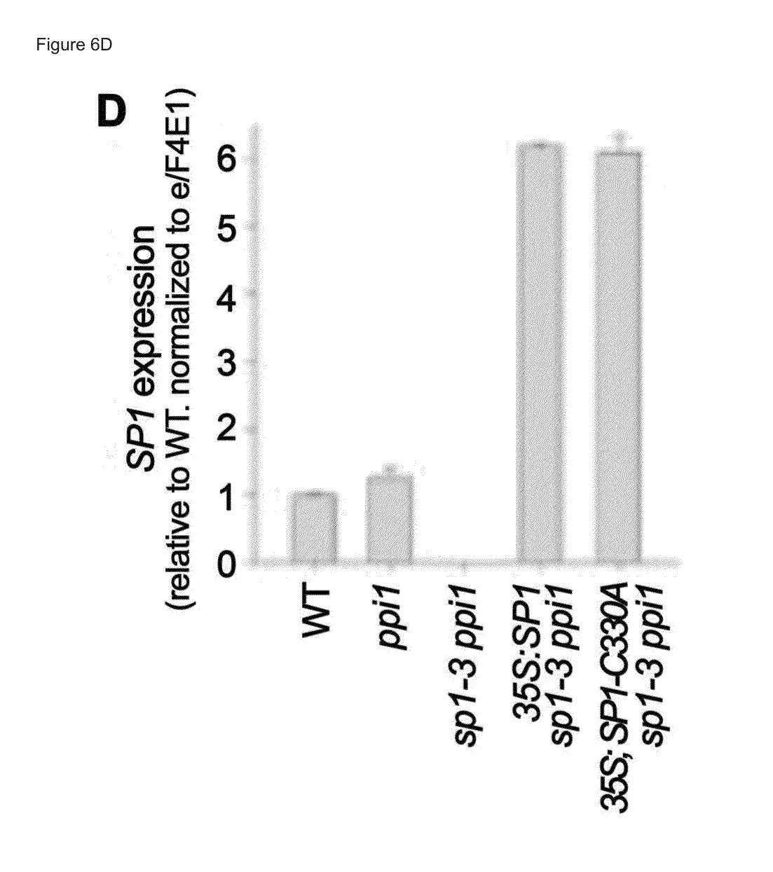

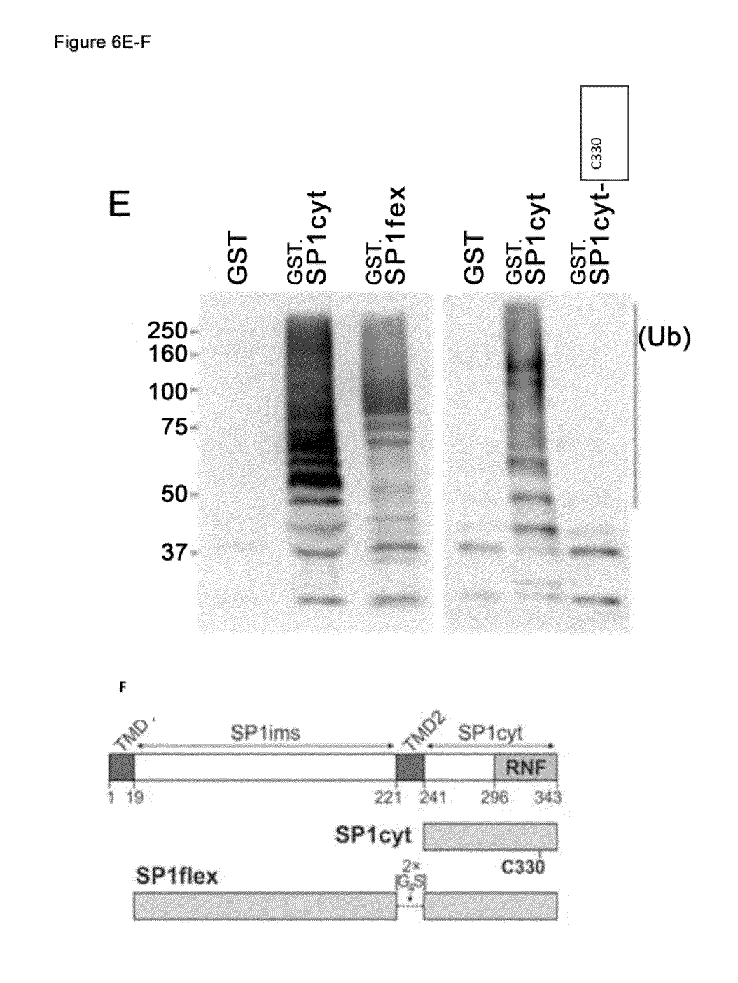

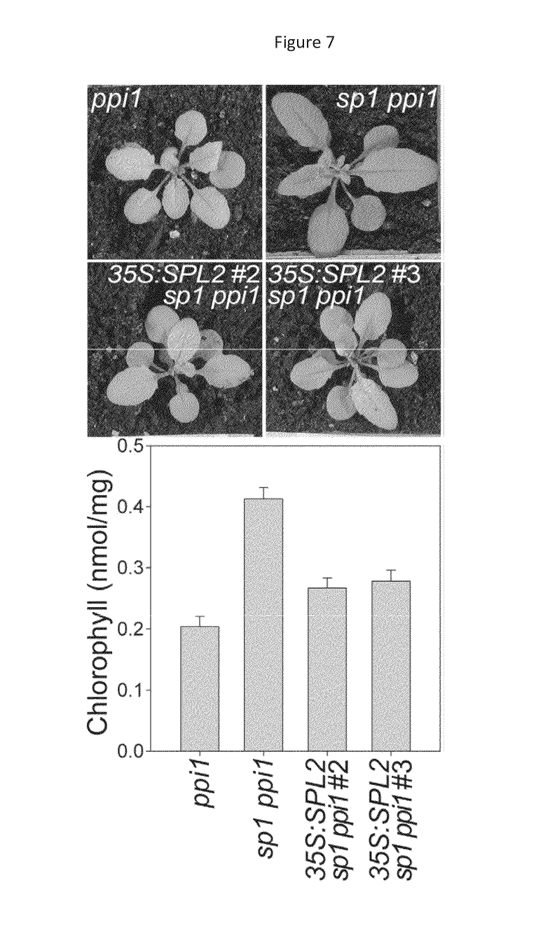

We identified the SP1 locus (At1g63900) by map-based cloning; the original sp1-1 allele carries a splice-junction mutation, causing frame-shifts, while two insertional mutants (sp1-2 and sp1-3) also lack the native SP1 transcript and are phenotypically similar. SP1 is a putative C3HC4-type really interesting new gene (RING) ubiquitin E3 ligase (13, 14). Such E3s perform a crucial role in the ubiquitin-proteasome system (UPS), along with E1 and E2 enzymes and the 26S proteasome. The UPS is a central proteolytic system in eukaryotes with numerous components, accounting in Arabidopsis for ˜6% of the proteome (15). The E1, E2 and E3 enzymes cooperate to attach ubiquitin to target proteins, which typically are then degraded by the proteasome. Targets are identified primarily by the E3s, of which there are many (˜90% of 1,600 UPS components in Arabidopsis are E3s), enabling specific recognition (and regulation) of numerous, functionally-diverse substrates (15). Areas of UPS activity include the nucleus, cytosol, endoplasmic reticulum, and the mitochondrial outer membrane (15, 18, 19). While un-imported plastid pre-proteins in the cytosol are UPS substrates (21, 31), the plastid itself was not previously recognized as a target. Overexpression of SP1 accentuated the phenotypes of TOC mutants, supporting the notion that it regulates the import machinery (FIG. 5).

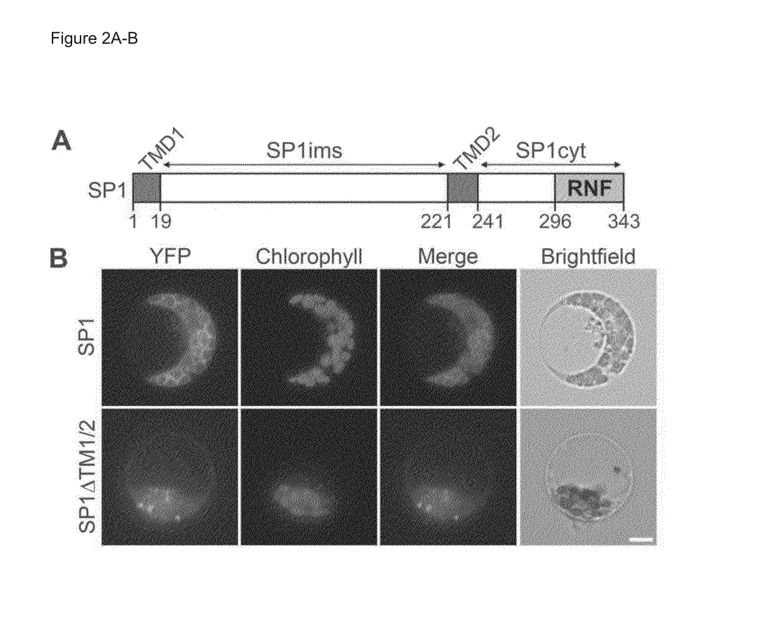

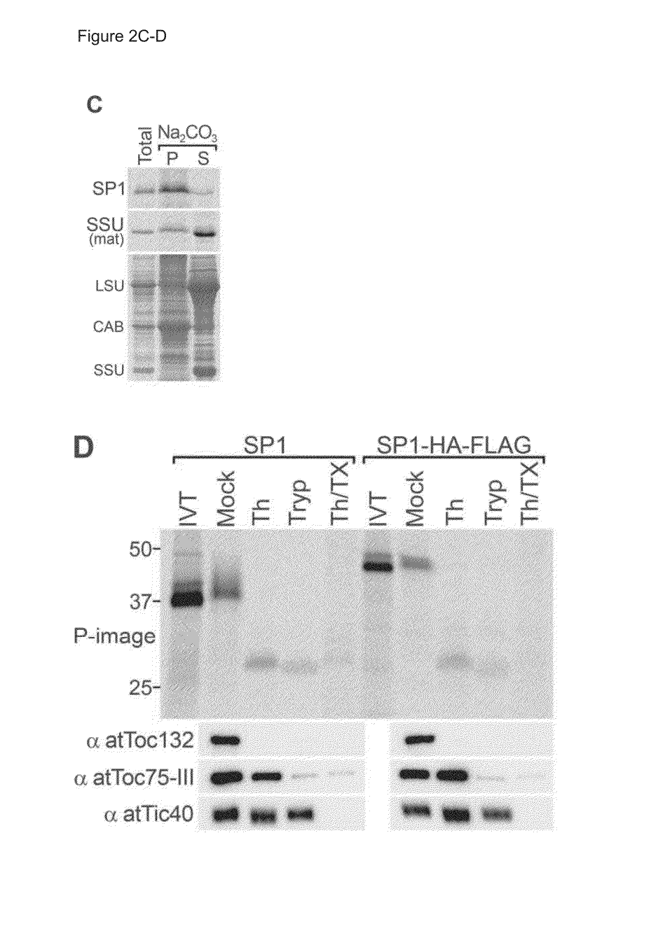

The SP1 protein has two predicted transmembrane spans (FIG. 2A). Translational fusions to yellow fluorescent protein (YFP) indicated localization to the chloroplast envelope that was dependent upon these transmembrane domains (FIG. 2B). In isolated chloroplasts, SP1 was resistant to alkaline extraction and partially sensitive to applied proteases (FIGS. 2, C and D), indicating that it is an integral outer membrane protein with an intermembrane-space domain, and that the RING domain is cytosolically-oriented and accessible to UPS components. Localization of SP1 to chloroplasts (a major source of reactive oxygen species) may explain previous results linking SP1 to programmed cell death.

Two SP1 homologues exist in Arabidopsis: SP1-Like1 (SPL1) and SPL2. They share topological similarity and considerable sequence identity with SP1, and both were located in the chloroplast envelope. Overexpression of SPL2 did complement sp1. However, overexpression of SPL1, did not complement sp1. Also related to SP1 is the human mitochondrial outer membrane protein MULAN/MAPL, which is reported to control mitochondrial dynamics.

In planta activity of SP1 depended on the presence of a functional RING domain (FIG. S7, A to D), which in E3s is required for E2 recruitment (14, 15). Purified SP1 had self-ubiquitination activity (13), as is typical for E3s, and this was similarly dependent upon RING functionality (FIGS. 5, E and F). Polyubiquitinated SP1 was also detected in plants, in amounts proportional to RING integrity. Such auto-ubiquitination implies that SP1 itself is subject to UPS control, as E3s frequently are (14). Accordingly, cellular SP1 protein levels were elevated upon treatment with the proteasome inhibitor MG132.

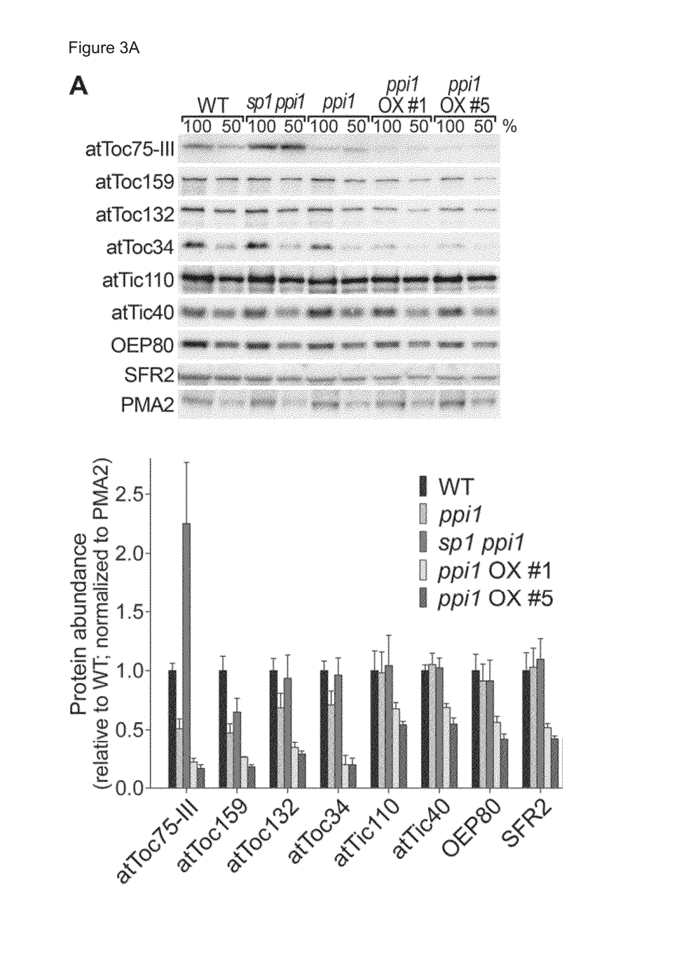

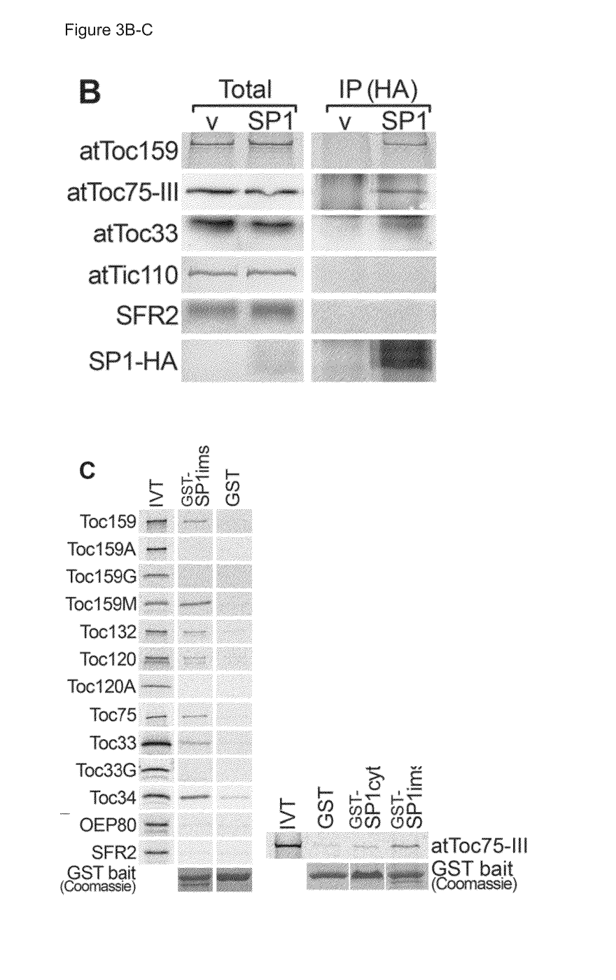

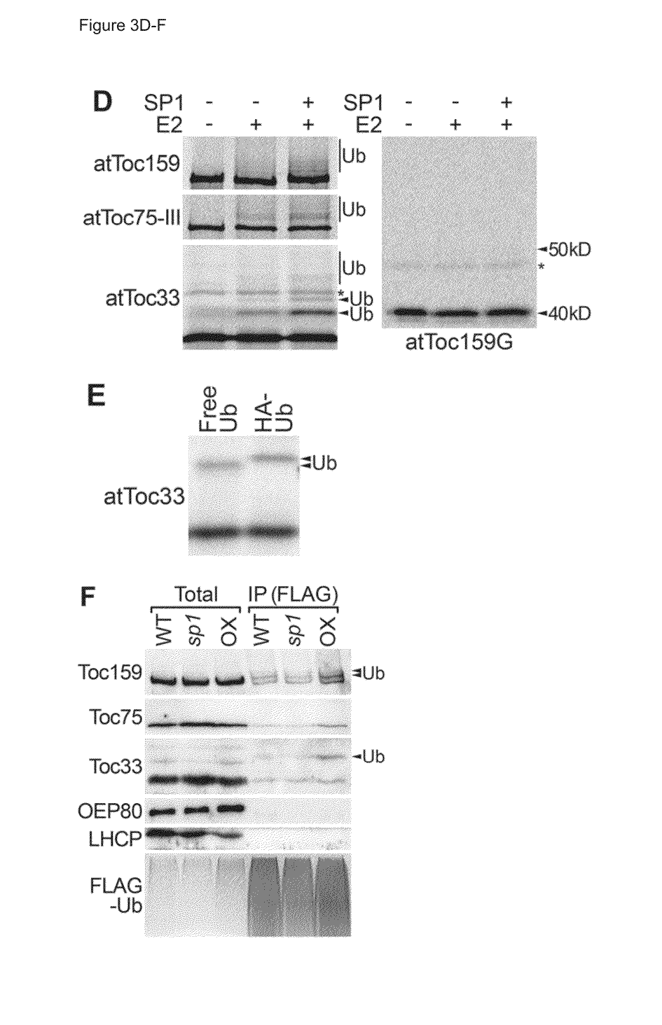

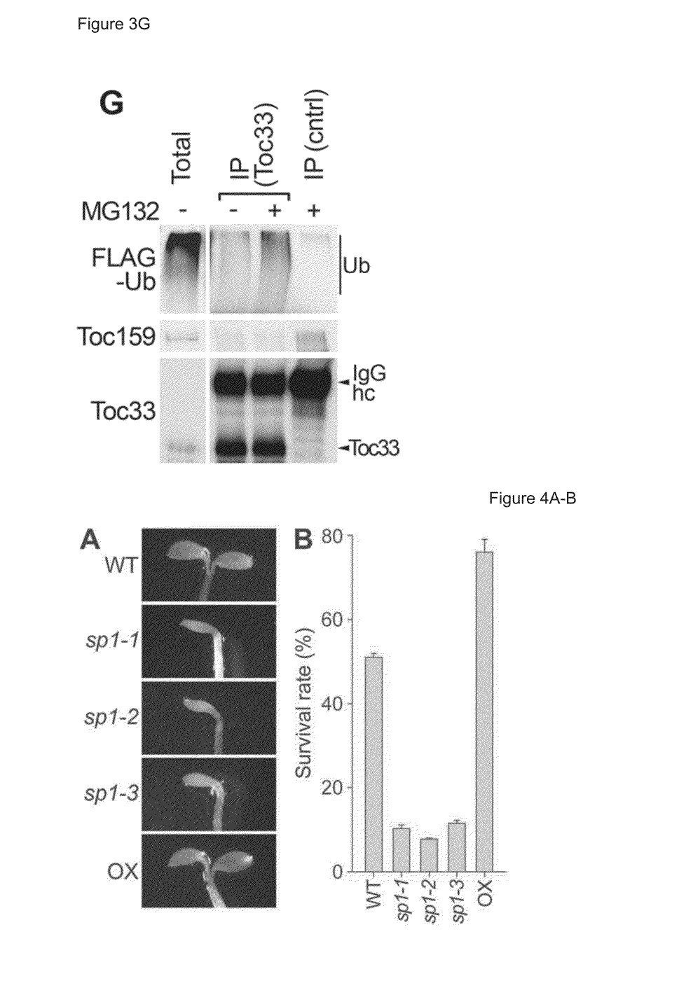

Phenocopy of sp1-mediated suppression was observed when 26S proteasome mutants were crossed to ppi1 or toc75-III-3, suggesting that the UPS indeed controls chloroplast development. We therefore set about identifying the target(s) of SP1 E3 activity. All tested TOC proteins were deficient in ppi1 relative to wild type, but substantially recovered in sp1 ppi1 (FIG. 3A); other envelope proteins (Tic110, Tic40, OEP80 and SFR2) were largely unaffected by sp1. These changes were not attributable to pre-translational events, as TOC transcript levels were comparable in the different genotypes. Similar TOC protein abundance recovery was apparent in sp1 toc75-III-3. Significantly, TOC protein levels were also elevated in the visibly-normal sp1 single mutant, arguing against the possibility that the protein changes in sp1 ppi1 and sp1 toc75-III-3 were a consequence (rather than a cause) of the phenotypic recovery. Moreover, SP1 overexpression preferentially depleted TOC proteins (FIG. 3A); effects on other envelope proteins in the ppi1 background were likely indirect consequences of general phenotype severity (FIG. 3A; FIG. 5).

Consistent with the notion that TOC proteins are targeted for UPS-mediated degradation by SP1, all three principal TOC components co-immunoprecipitated with epitope-tagged SP1 from plant extracts (FIG. 3B). In vitro pull-down experiments revealed SP1 interactions with Toc75 and all tested TOC receptors (FIG. 3C), which is not unusual as E3s often have diverse substrates (14, 15). These interactions were mediated primarily by the SP1 intermembrane-space domain and the membrane/intermembrane-space domains of the receptors.

In vitro ubiquitination assays using radiolabelled TOC proteins, purified SP1, and UPS components revealed high-molecular-weight species indicative of TOC ubiquitination (FIG. 3D). Some ubiquitination occurred in the absence of E3 (presumably mediated by E2 alone, which is not unexpected), but for each TOC substrate the extent of ubiquitination was enhanced in the presence of SP1. The identity of mono-ubiquitinated atToc33 was confirmed by a size shift upon utilization of different forms of ubiquitin (FIG. 3E).

In an in vivo assay for ubiquitination, the three main TOC components all co-immunoprecipitated with epitope-tagged ubiquitin, in amounts proportional to the expression of SP1 (in sp1, amounts were less than in wild type; in an SP1 overexpressor, amounts were more) (FIG. 3F). Moreover, modified forms of atToc159 and atToc33 were apparent in the precipitates, which likely correspond to ubiquitinated species. Absence of clearly ubiquitinated forms of Toc75 may indicate that it is less readily ubiquitinated than the receptors in vivo, or more readily de-ubiquitinated. Regardless, its association with other ubiquitinated TOC proteins may be sufficient to promote its turnover through in-trans action of the UPS. In a reciprocal experiment (performed under denaturing conditions to disrupt non-covalent protein-protein associations; see atToc159 control), high-molecular-weight ubiquitin smears were apparent in atToc33 immunoprecipitates (FIG. 3G). Abundance of polyubiquitinated atToc33 was controlled by proteasomal activity, as revealed by MG132 treatment. We conclude that TOC components are indeed ubiquitinated in vivo, and that this controls their turnover. Genetic suppression by sp1 is likely due to the stabilization of TOC components (e.g., atToc75-III and atToc34).

Our data imply a role for SP1 in the reorganization of the TOC machinery, and a new mechanism for the regulation of plastid biogenesis. This might be important during developmental phases in which plastids convert from one form to another through organellar proteome changes (1-3). A commercially important plastid transition occurs during fruit ripening in crops such as tomato and citrus: chloroplasts differentiate into chromoplasts which accumulate carotenoid pigments of dietary significance (3). In Arabidopsis, a striking example occurs when etiolated seedlings are exposed to light, whereupon heterotrophic etioplasts rapidly differentiate into chloroplasts. This is essential for initiation of photoautotrophic growth after seed germination beneath the soil. In accordance with the hypothesis, sp1 single mutants de-etiolated inefficiently, displaying reduced survival rates linked to delayed organellar differentiation (FIG. 4, A to E), reduced accumulation of photosynthetic proteins, and imbalances in TOC receptor levels. At the other end of the life-cycle, chloroplasts transform into gerontoplasts as catabolic enzymes accumulate to recover resources from the organelles of senescent leaves for use elsewhere in the plant. This response is characterised by declining photosynthetic performance, and can be induced prematurely by dark treatment. The sp1 mutation also attenuated this transition (FIGS. 4, F and G), while SP1 overexpression enhanced both senescence and de-etiolation (FIG. 4), presumably due to the hastening of organellar proteome changes.

### Example 2

### Expression of solSP1

SP1 homologues are widely distributed in plants. To assess whether SP1's proposed role in plastid developmental transitions is conserved, we have studied the tomato SP1 orthologue, solSP1 (Solyc06g084360.1.1; 73% a.a. identity to SP1, SEQ ID No. 9), in tomato. A particular advantage of working with tomato will be the opportunity to study SP1's importance for the differentiation of carotenoid-rich chromoplasts from chloroplasts during tomato fruit ripening, which is not possible in Arabidopsis.

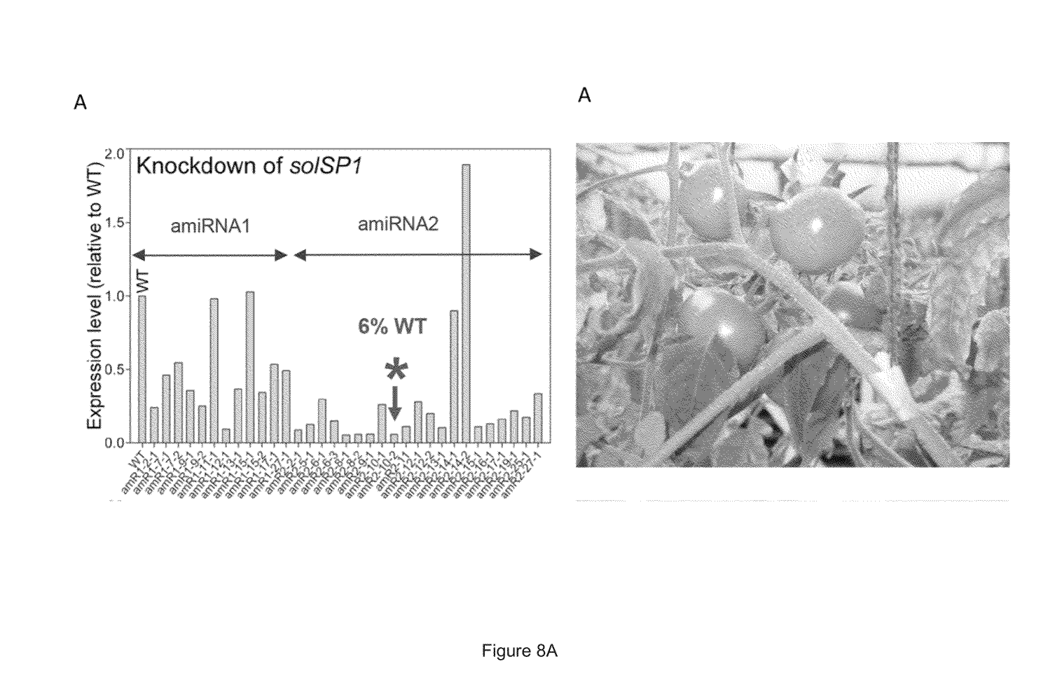

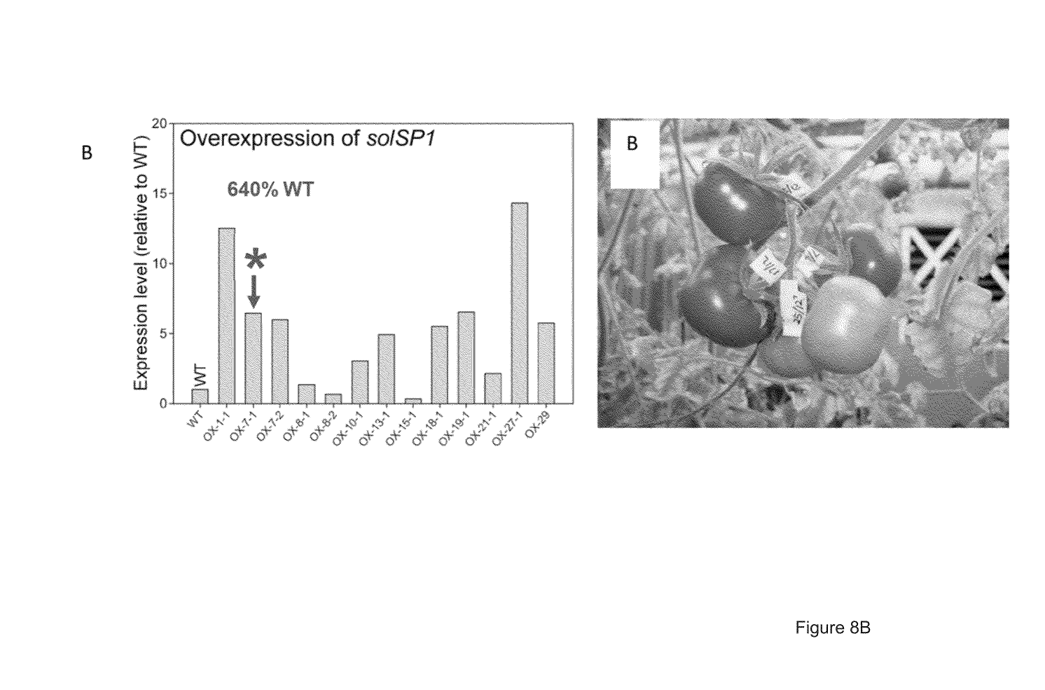

We have confirmed that solSP1 localizes to the plastid envelope using asolSP1-YFP fusion (made in p2GWY7) using the Nikon Eclipse TE-2000E system (as for Arabidopsis SP1) in transfected tomato leaf protoplasts. We have generated transgenic tomato plants that either silence (knockdown, KD) or overexpress (OX) solSP1. For KD, we have made two different artificial microRNA (amiRNA) constructs based on pRS300 that target different regions of the gene. Together with full-length solSP1 cDNA (for OX), these were cloned into pK7WG2D to be driven by the 35S promoter; this vector also contains a GFP marker to facilitate early confirmation of transformants. Transformants have been tested by RT-PCR to determine the extent of KD and OX. T0 generations have been studied, as follows: Fruit phenotype has been compared visually at different stages of ripening (relative to WT). Preliminary data indicates that fruit ripening is delayed in solSP1 KD lines and accelerated in OX lines (see FIG. 8).

Underlined sequences in SEQ ID NO. 11 represent target sequence for amRMA construction. The following amRNAs were used:

AmiRNA-1:
(SEQ ID No. 57)
TCATATGACCACACGCGACAA
AmiRNA-2:
(SEQ ID No. 58)
TAAGTAGATATACACTGACAG

### Example 3

### SP1 Regulates Stress Tolerance

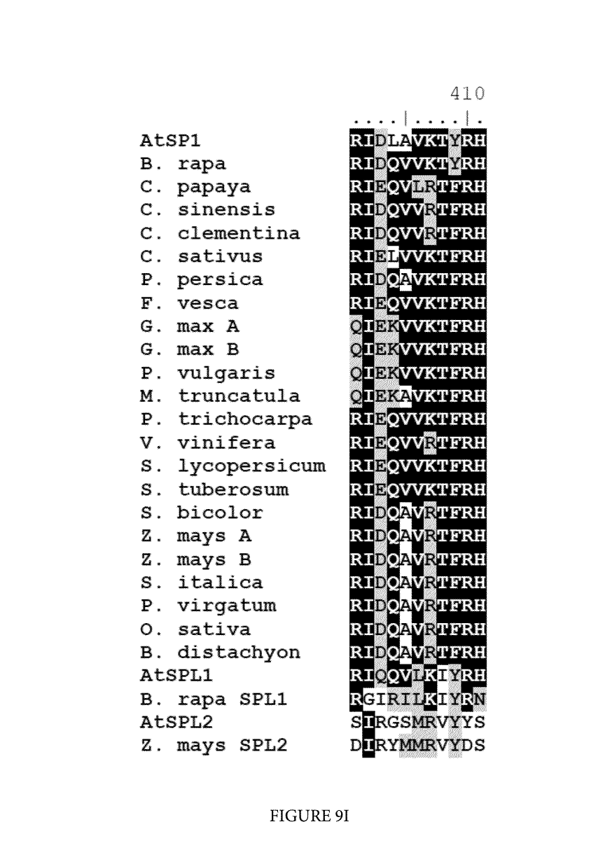

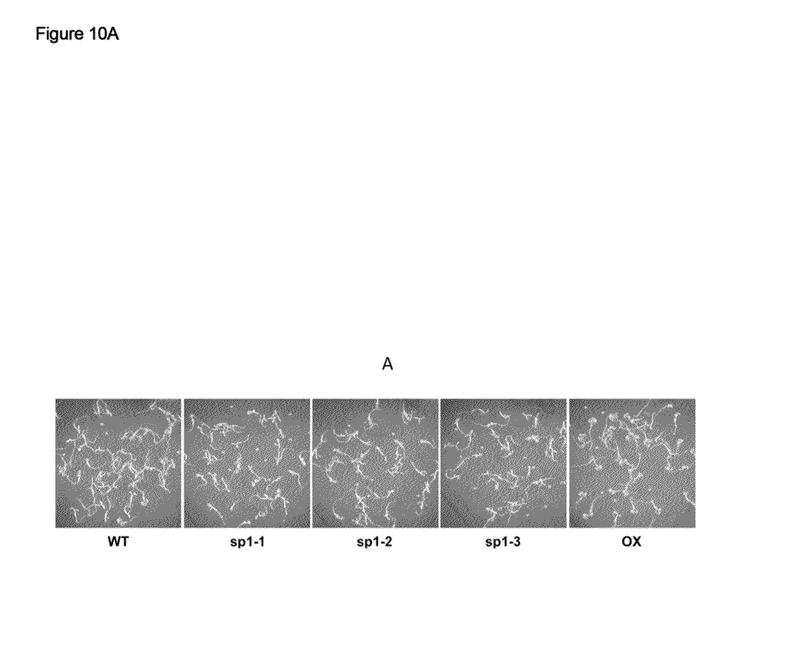

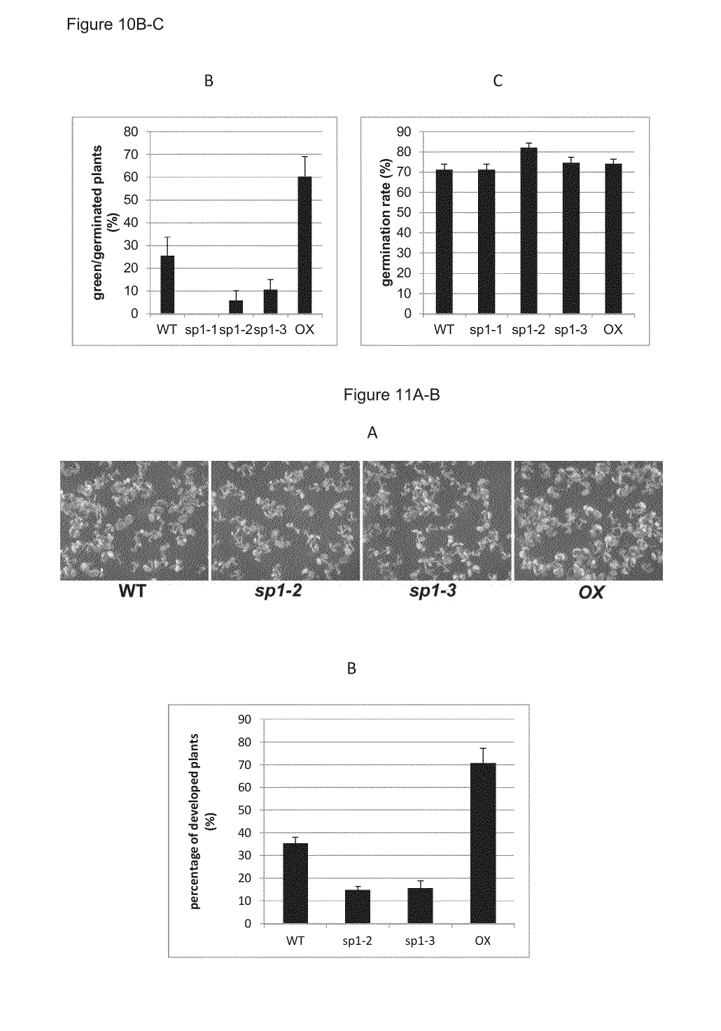

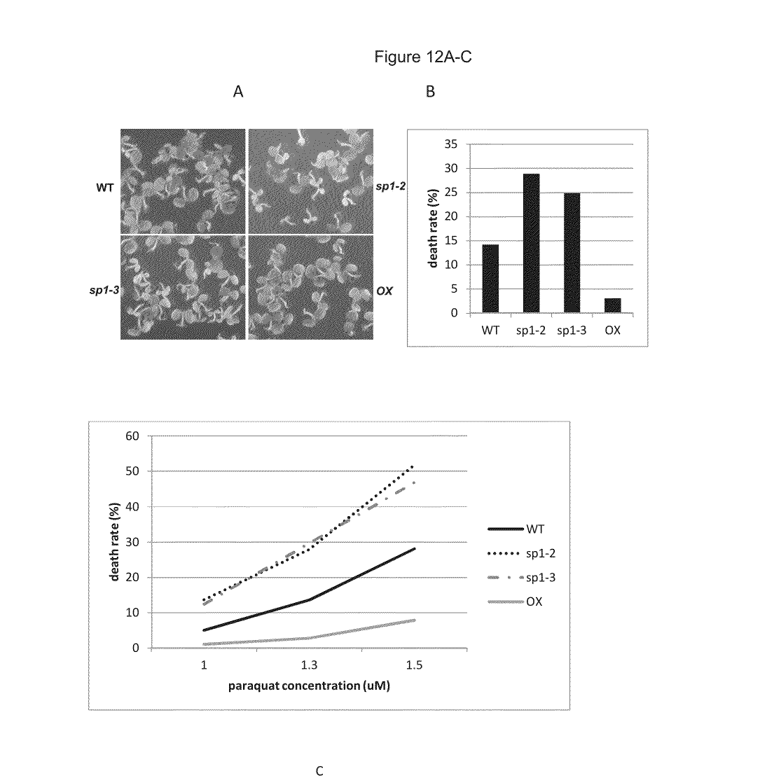

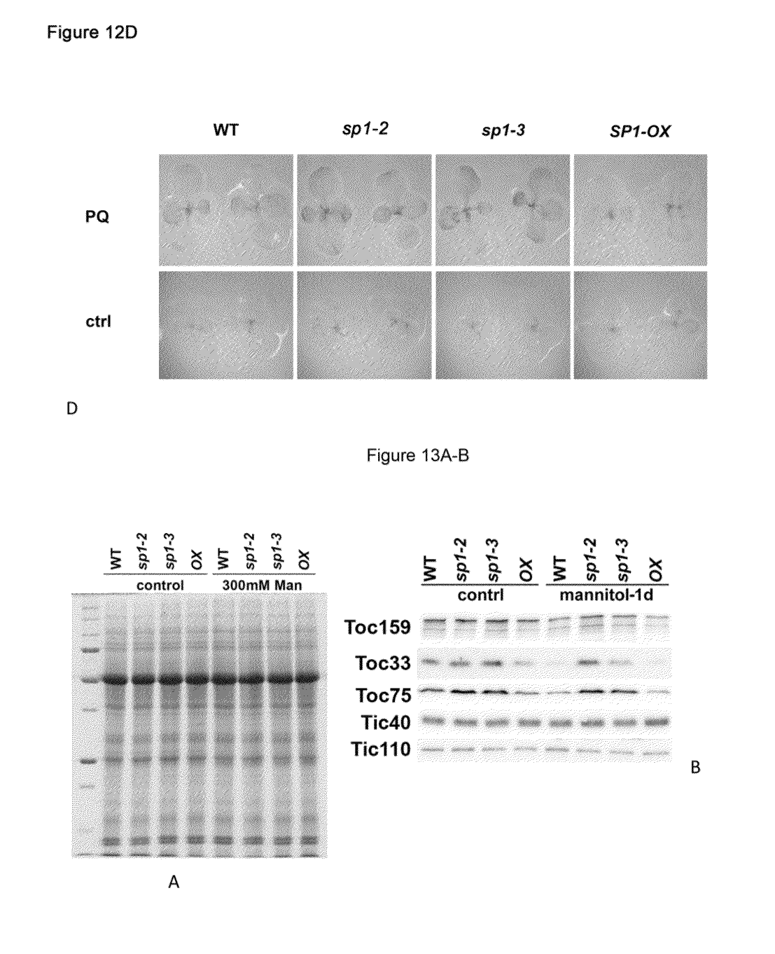

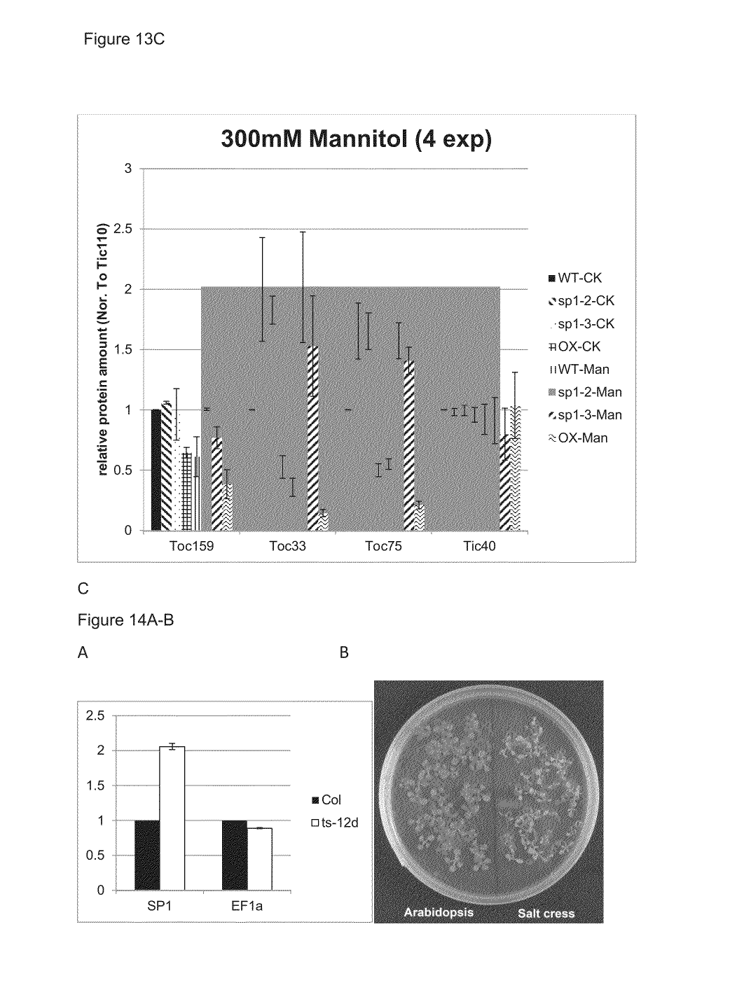

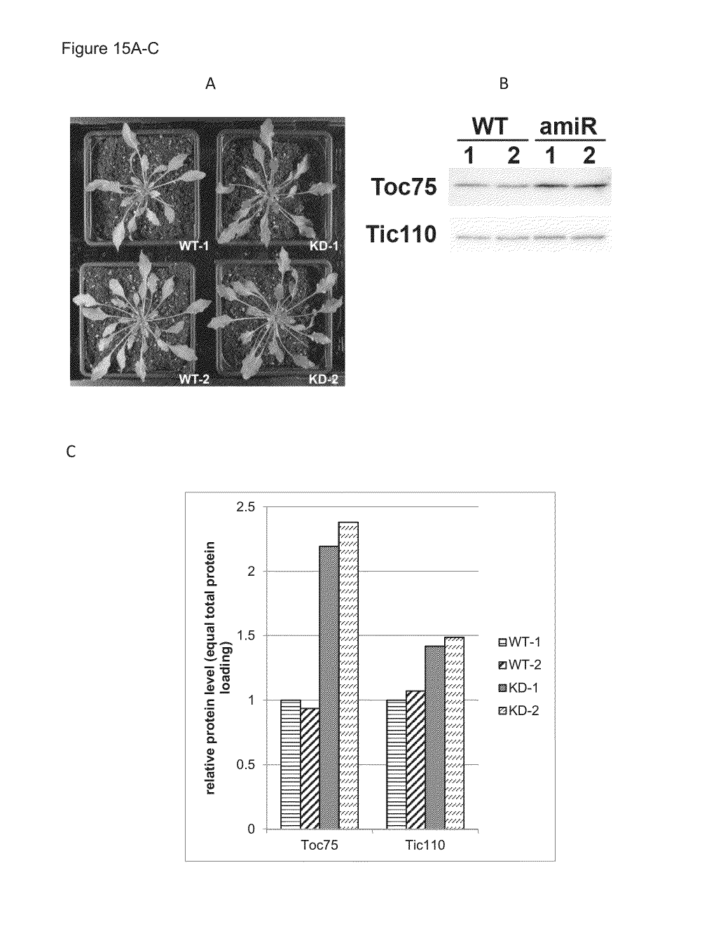

Regulation of stress tolerance by SP1 is shown in FIGS. 10-15. We have shown that Arabidopsis sp1 mutants do not green well following germination on saline medium (150 mM NaCl), relative to wild-type plants, whereas plants overexpressing SP1 show considerable improvements in greening under these conditions.

Similarly, Arabidopsis sp1 mutant plants do not develop under osmotic stress conditions (400 mM mannitol), whereas plants overexpressing SP1 exhibit improved development relative to the wild type under these conditions.

In response to oxidative stress (brought about by treatment with the herbicide paraquat), sp1 mutant Arabidopsis plants exhibit a higher rate of death than the wild type, whereas the SP1 overexpressor plants exhibit a significantly reduced death rate relative to wild type (i.e., the SP1 overexpressor plants display considerably improved survival under oxidative stress conditions).

Staining of seedlings exposed to oxidative stress with diaminobenzidine (DAB; which detects hydrogen peroxide) revealed that the aforementioned enhanced survival rate of SP1 overexpressor plants is correlated with reduced accumulation of reactive oxygen species.

We have shown that when Arabidopsis seedlings are exposed to osmotic stress for two days, the levels of TOC proteins (components of the plastid protein import machinery in the plastid outer membrane) decline rapidly—more so than other tested proteins. This effect on TOC protein levels is dependent upon SP1 activity, as the response was attenuated in sp1 mutant plants. It is possible that this SP1-mediated response is designed to limit the import of photosynthetic apparatus components into plastids, in order to limit photosynthetic activity and thus the potential for damaging ROS accumulation under stress conditions.

In salt cress (Thelungiella salsuginea)—a close relative of Arabidopsis that is naturally tolerant of abiotic stresses such as high salinity—the expression of SP1 is significantly elevated, relative to Arabidopsis. Interestingly, salt cress plants also have a characteristic pale-green appearance, somewhat like that of TOC-deficient mutants of Arabidopsis.

We have shown that a reduction in the expression of the salt cress SP1 gene, using amiRNA technology, results in transgenic plants that are visibly greener than wild-type salt cress plants. This visible appearance change was correlated with elevated levels of the core TOC protein Toc75.

TABLE 1
Primers used during the course of the study.

| Primer name | Primer sequence (5′ to 30′)* | Used to generate . . . |
|---|---|---|
| SP1-CDS-F | AAAAAGCGGCTTCATGATTCCTTGGGGTGGAG | . . . SP1 CDS for |
| | SEQ ID No. 12 | complementation, GST fusions, |
| | | and C-terminal fusions |
| SP1- | AGAAAGCTGGGTTTCAGTGACGATATGTCTTAACC | . . . SP1 CDS for |
| CDS-R | SEQ ID No. 13 | complementation ad GST |
| | | fusions |
| SP1- | AGAAAGCTGGGTTGTGACGATATGTCTTAACC | . . . SP1 CDS for C-terminal |
| nonstop-R | SEQ ID No. 14 | fusions |
| SP1- | GACGATATGTCTTAACCGCCAGATCTATTCGTCTCC | . . . RNF point mutation |
| C330A-R | GAGCAAGTG | |
| | SEQ ID No. 15 | |
| SP1- | AAAAAGCAGGCTCCATGCGGAGTAGTGGCAGGGAT | . . . variants lacking TMSs |
| ΔTM1-F | SEQ ID No. 16 | for GST and YFP fusions |
| SP1- | TAGAACAGAGTCAATTTTGTACAACCTTGACCATTT | |
| ΔTM2-R | TC | |
| | SEQ ID No. 17 | |
| SP1- | TCAAGGTTGTACAAAATTGACTCTGTTCTAGAGAG | |
| ΔTM2-F | SEQ ID No. 18 | |
| SP1- | CTCCACCGCTACCGCCGCCTCCTTTGTACAACCTTG | . . . construct in which |
| ΔTM2-Flex1 | ACC | TMD2 is replaced by a |
| | SEQ ID No. 19 | flexible linker, for GST |
| SP1- | GCGGTAGCGGTGGAGGTGGCAGCATTGACTCTGTTC | fusion |
| ΔTM2-Flex2 | TAGAG | |
| | SEQ ID No. 20 | |
| SP1- | GGATCCCGGAGTAGTGGCAGGGAT | . . . GST-SP1 ims fusion for |
| pGEX-IMS-F | SEQ ID No. 21 | bacterial expression |
| SP1- | AAGCTTTTTGTACAACCTTGACCATTTTC | |
| pGEX-IMS-R | SEQ ID No. 22 | |
| SP1- | AAAAAGCAGGCTCCATTGACTCTGTTCTAGAGAG | . . . GST-SP1 cyt fusion for |
| cyt-F | SEQ ID No. 23 | bacterial expression |
| SPL1- | AAAAAGCAGGCTCCATGATACATTTGGCTGGATT | . . . full-length SPL1 CDS |
| CDS-F | SEQ ID No. 24 | for complementation |
| SPL1- | AGAAAGCTGGGTTTCAATGGCGGTAAATTTTCAAAA | |
| CDS-R | C | |
| | SEQ ID No. 25 | |
| SPL1- | AGAAAGCTGGGTTATGGCGGTAAATTTTCAAAAC | . . . SPL1 CDS for YFP fusion |
| nonstop-R | SEQ ID No. 26 | |
| SPL2- | AAAAAGCAGGCTCCATGTCCTCGCCGGAGCGTG | . . . SPL2 CDS for YFP fusion |
| CDS-F | SEQ ID No. 27 | |
| SPL2- | AGAAAGCTGGGTTAGAGTAATATACACGCATAG | |
| nonstop-R | SEQ ID No. 28 | |
| YFP-F | AAAAAGCAGGCTCCATGGTGAGCAAGGGCGAG | . . . YFP CDS for HA fusion |
| | SEQ ID No. 29 | |
| YFP- | AGAAAGCTGGGTTCTTGTACAGCTCGTCCATG | |
| nonstop-R | SEQ ID No. 30 | |

### REFERENCES

1. E. López-Juez, K. A. Pyke, Int. J Dev. Biol. 49, 557 (2005).
2. T. Kleffmann et al., Plant Physiol. 143, 912 (2007).
3. C: Barsan et al., J. Exp. Bot. 61, 2413 (2010).
4. H. M. Li, C. C. Chiu, Annu. Rev. Plant Biol. 61, 157 (2010).
5. P. Jarvis, New Phytol. 179, 257 (2008).
6. F. Kessler, D. J. Schnell, Traffic 7, 248 (2006).
7. P. Jarvis et al., Science 282, 100 (1998).
8. J. Bauer et al., Nature 403, 203 (2000).
9. S: Kubis et al., Plant Cell 15, 1859 (2003).
10. Y. Ivanova, M. D. Smith, K. Chen, D. J. Schnell, Mol. Biol. Cell 15, 3379 (2004).
11. J. P. Stanga, K. Boonsirichai, J. C. Sedbrook, M. S. Otegui, P. H. Masson, Plant Physiol. 149, 1896 (2009).
12. W. Huang, Q. Ling, J. Bédard, K. Lilley, P. Jarvis, Plant Physiol. 157, 147 (2011).
13. S. L. Stone et al., Plant Physiol. 137, 13 (2005).
14. R. J. Deshaies, C. A. Joazeiro, Annu. Rev. Biochem. 78, 399 (2009).
15. R D. Vierstra, Nat. Rev. Mol. Cell Biol. 10, 385 (2009).
16. S. S. Vembar, J. L. Brodsky, Nat. Rev. Mol. Cell Biol. 9, 944 (2008).
17. N. Livnat-Levanon, M. H. Glickman, Biochim. Biophys. Acta 1809, 80 (2011).
18. G. Shen, Z. Adam, H. Zhang, Plant J. 52, 309 (2007).
19. S. Lee et al., Plant Cell 21, 3984 (2009).
20. B. M. Vindhya et al., Plant Cell Rep. 30, 37 (2011).
21. W. Li et al., PLoS ONE 3, e1487 (2008).
22. B. Zhang et al., Cell Res. 18, 900 (2008).
23. E. Braschi, R Zunino, H. M. McBride, EMBO Rep. 10, 748 (2009).
24. J. Myung, K. B. Kim, C. M. Crews, Med. Res. Rev. 21, 245 (2001).
25. W. Huang et al., Plant Cell 18, 2479 (2006).
26. D. Lu et al., Science 332, 1439 (2011).
27. S. Xu, G. Peng, Y. Wang, S. Fang, M. Karbowski, Mol. Biol. Cell 22, 291 (2011).
28. S. Prakash, T. Inobe, A. J. Hatch, A. Matouschek, Nat. Chem. Biol. 5, 29 (2009).
29. N. Mochizuki, R. Susek, J. Chory, Plant Physiol. 112, 1465 (1996).
30. S. Schelbert et al., Plant Cell 21, 767 (2009).
31. Basnayake B M, Li D, Zhang H, Li G, Virk N, Song F. Plant Cell Rep. 2011 January; 30(1):37-48.

Sequence listing
AtSP1 nucleic acid sequence; genomic, exons are marked in upper case
SEQ ID No. 1
ATGATTCCTTGGGGTGGAGTTACTTGCTGCCTCAGCGCCGCTGCTCTTTATCTTCTCGGCCGGAGTAGTGGCAGgtttgtctgatctattta
tatttcatcttcccaaagagattatcaatcaatcaaatcctttcttatccttttgagtgcagGGATGCTGAAGTACTCGAAACAGTCACTAG
GGTTAATCAGCTCAAGGAGTTAGgtaatcttcttctcccctgattgcttcatctactctcaggatgaagttttgatcatgttttctgattgt
tctgtatgtgtagCTCAATTGCTAGAATTAGATAGCAAGATTCTGCCTTTCATTGTTGCGGTATCAGGAAGAGTCGGCTCTGAGACACCTAT
CAAATGCGAGCATAGTGGCATACGCGGTGTTATTGTTGAAGAAACGgtatgttgtagactgatgattagcgcatggaacttagtttgttttc
tggtttaatcgattaggttttatgtgaaactctagtaattgcatgatcttttcagGCGGAACAACATTTCCTGAAACATAATGAGACGGGTT
CTTGGGTACAAGATAGTGCGTTGATGCTATCAATGAGCAAAGAGGTTCCTTGGTTTCTGgtaagtctagtctagtagcatagtgtttgaaac
aactgtgattggatgttttcttaataccatttccaaaactgtttgggatagGACGATGGGACAAGCCGTGTCCATGTAATGGGAGCTCGTGG
TGCGACGGGTTTTGCCTTGACTGTTGGTAGTGAAGTTTTTGAAGAGTCAGGACGCTCGCTTGTACGAGGAACACTTGATTATCTTCAAGGAC
TTAAGgtttttacttttctttccggttctttgtttgttggcttctctatttgcttaagcggccattgttttgtttcagATGCTTGGAGTTAA
GCGCATTGAGCGTGTTCTTCCAACTGGAATACCGCTAACAATTGTTGGTGAGgtatgtcgtattctcagtgttttcgggtcctctcttttgc
ttaagttgtaactgttgatagagatacatagcacactaactccttcatcagtctggtatttgcctcttgaaattttctcaaagttcctttaa
tagcaatatttgtaggaagtgggattgatctatgtatagaggcttaccgatgagtttaaatctaatttgtgttgctgccatgtataacagGC
TGTCAAGGACGATATTGGAGAATTCAGGATTCAAAAACCTGACAGGGGCCCTTTCTACGTCTCTTCTAAATCACTCGATCAGCTCATTTCTA
ATTTGGGAAAATGGTCAAGgtcgtgtctctctcctctctcggttcttctcctatactcttgtagaaaaacggcaatgagccaaactgattga
gaagagtataatttacagGTTGTACAAATATGCCTCCATGGGTTTTACTGTTCTTGGTGTGTTCCTAATTACGAAGCATGTCATTGACTCTG
TTCTAGAGAGAAGACGGCGGAGACAGTTACAAAAAAGgtatgtcacagatttgtctgtctaaaagtgaataaccgttctcaagcatgagtac
tagatcggcttgtttctctcgaaactatgtacacacaaaattaagtagtcagctgtttttgcagAGTGCTTGACGCAGCAGCAAAGAGAGCT
GAGCTAGAGAGTGAAGgtatccattggtgaatctattattctacatataggttgcactggctctgactacaatctcttctgaccagGTTCAA
ACGGGACACGTGAGAGCATTTCAGATTCTACCAAGAAAGAAGACGCTGTTCCTGATCTCTGTGTGATATGCCTAGAGCAGGAGTACAACGCT
GTGTTTGTCCCgtaagcattcttccgccatttttggttgattctgcatttgcaacttgctaaaatgcttgtggttggtactcgcagGTGTGG
TCATATGTGCTGCTGCACCGCATGCTCCTCCCACTTGACCAGCTGTCCACTTTGTCGGAGACGAATAGATCTGGCGGTTAAGACATATCGTC
ACTGA
AtSP1 nucleic acid sequence; cDNA, accession number NM_105064
SEQ ID No. 2
atgagaatattgagagagatcgaagcaaaggatcattcaattccaaccctctgaatcttttaatttcccctttcgaaattctcctcttcttt
cactgcttctagtttctaattcttcaaactcttcctcgattcatactcataactctcattagctaatttcgcatgatcttcttccatctctc
tgtgttctaaatccagattcgtttcactcccatctctatttcattcaattcgctgcatccagattcaaaacctacctctatctctctgctca
tcaataacttcaaaggtattgttgttcttctgcaaacaagtaagagtgacttcagagtctgatgattccttggggtggagttacttgctgcc
tcagcgccgctgctctttatcttctcggccggagtagtggcagggatgctgaagtactcgaaacagtcactagggttaatcagctcaaggag
ttagctcaattgctagaattagatagcaagattctgcctttcattgttgcggtatcaggaagagtcggctctgagacacctatcaaatgcga
gcatagtggcatacgcggtgttattgttgaagaaacggcggaacaacatttcctgaaacataatgagacgggttcttgggtacaagatagtg
cgttgatgctatcaatgagcaaagaggttccttggtttctggacgatgggacaagccgtgtccatgtaatgggagctcgtggtgcgacgggt
tttgccttgactgttggtagtgaagtttttgaagagtcaggacgctcgcttgtacgaggaacacttgattatcttcaaggacttaagatgat
ggagttaagcgcattgagcgtgttatccaactggaataccgctaacaattguggtgaggctgtcaaggacgatattggagaattcaggattc
aaaaacctgacaggggccattctacgtctcttctaaatcactcgatcagctcatttctaatttgggaaaatggtcaaggttgtacaaatatg
cctccatgggttttactgttcttggtgtgttcctaattacgaagcatgtcattgactctgttctagagagaagacggcggagacagttacaa
aaaagagtgcttgacgcagcagcaaagagagctgagctagagagtgaaggttcaaacgggacacgtgagagcatttcagattctaccaagaa
agaagacgctgttcctgatctctgtgtgatatgcctagagcaggagtacaacgctgtgtttgtcccgtgtggtcatatgtgctgctgcaccg
catgctcctcccacttgaccagctgtccactttgtcggagacgaatagatctggcggttaagacatatcgtcactgaacaacaactcaggcc
tcagaaacattctctacttgagtatgtctgtaaataccgcaaaatcaaaacattacacagtttagcgttcgatattccctttggtttgattt
cgacaacaaaacattttgaattatatagaaacataaggtgtttactcgatttgcaaaacagtacattcgtgtttacttattcgtgttgttgc
caatgccatgaggtg
AtSP1 peptide sequence
SEQ ID No. 3
MIPWGGVTCCLSAAALYLLGRSSGRDAEVLETVTRVNQLKELAQLLELDSKILPFIVAVSGRVGSETPIKCEHSGIRGVIVEETAEQHFLKH
NETGSWVQDSALMLSMSKEVPWFLDDGTSRVHVMGARGATGFALTVGSEVFEESGRSLVRGTLDYLQGLKMLGVKRIERVLPTGIPLTIVGE
AVKDDIGEFRIQKPDRGPFYVSSKSLDQLISNLGKWSRLYKYASMGFTVLGVFLITKHVIDSVLERRRRRQLQKRVLDAAAKRAELESEGSN
GTRESISDSTKKEDAVPDLCVICLEQEYNAVFVPCGHMCCCTACSSHLTSCPLCRRRIDLAVKTYRH
AtSPL2 nucleic acid sequence; genomic, exons are marked in uppercase
SEQ ID NO. 7
ATGTCCTCGCCGGAGCGTGCTCTCTTGAATCTATTGACGGACATCGCTCTCTCCTTCGACGGCGCAATCCTCGGACTGACTTTAGCTGTTAG
CGCCGTCGGTTCTGCTCTCAAGTATGCTTCTACCAACGCCGCGTTGAAGAAGATCAAAGATGCACCCGAAGTCTCCATTTCCGACCTCCGAT
CACTGCTTCCGGCTTCTGAAGACAAATCAGAGACTAACGATAACAGGAAGAGCAATGATCAGCGAATCGTTGTGGTTCGCGGAGTCGTCAAA
CCGAAAATTTCCGGTGATGAGGGTTACAAGAACAACAACGTGTTAATCTCTCCGGAGACTGGAGATAAAGCTCTTATCATTCAGAGAACGCA
AACTgtacgttgctctaaaccctaagtgtttatgtttccctcaaattgcaattgacattgacctaattttgattgtgttatatcaaaggctt
aggttaaagagtttggatgatttatcatgattaggtgttcattgtgattgattaagttaggattatgtaagttggtactggtgtgatctcag
aagggaaatgtatgtttctaatgtcatcattttgttgttgacagTATGTGTATAGTGGATGGAAGAGATTGTTTCAGTCCACTGGTCACCGG
TTTATGTTGGAGAGATCATTGCGCAAACACGGTGCTGATTTTACGAGAACGgtaaagtctctctatcagcttcagagctttcaactttggct
ctgtatccatgatcaaacttgttttgtgtatcctttgctttgtgcagGTCCCATTTGTCATTGTTGGAAAGGATCAGCAATCGAACTCTAGT
TTTGTCGCTGTTAACATGGATGGCTCAAGGCAACCTCTTCCGCTTACCACTGTGTATAATCGCTTGCAGCCTATTAATTCTTCGTTTCTTCA
AGCCTTTTTGTACCCCGATTATCCTgtgagtatatatatattcaaagaaatttctggttttgaggaactgatggttacttgttatggctaac
tttgtaatcctttctcttccaagGTTGGTCTGCTTGATATAGAGAAGATTTTACCACCTGGGAAAGATATAACAGCTGTTGGCATCTACAGT
TTTAATAATGGAGTTCCTGAAATCAAATCGTGCCAAGATCTTCCTTATTTCTTgtgagattttatctgaatatttttcgttcttatatatct
gcctgcgttctagctcaaattattacttcatcttcttctttatgttttctcaatctgttgatcatttgacaagatttagtccattgttgcag
aaaggctctatttgctgttcatatataaatctttatgaataatctttgcagATCAGAGATGACCAAGGACAAGATGATTGAGGACCTTATGG
AACAGACCAACTTTATATTCTTGGGCAGCGTTATCTTGGGCATTGTGTCTGTTGGCATCCTTAGCTATGCTGCTGTCAGgtactggagttgt
gagagatcagatttggttttagatttgggcaatgtttcgatttgaaatctgtttaactgtcttatcttcagGACCTGGAATAAATGGAAACA
ATGGAACCATCAGAGAGAACTGCCACAGAGACCGAACGATTCTGTGGTCGATGATGAACCCGAAGATGCAGATGAAATCCCAGATGGAGAAT
TGTGTGTGATCTGCGTGTCAAGAAGGAGAGTACCTGCGTTTATTCCCTGTGGACATGTAGTATGTTGCAGGCGATGTGCTTCAACCGTGGAA
CGAGAGTTAAACCCTAAGTGTCCAGTTTGTCTTCAGAGCATTAGGGGATCTATGCGTGTATATTACTCTTAG
AtSPL2 nucleic acid sequence; cDNA
SEQ ID NO. 8
ACTTGTCCGTGTGACCGTGTCTACGACGTTGAAATCGAAATTACCACAATGTCCTCGCCGGAGCGTGCTCTCTTGAATCTATTGACGGACAT
CGCTCTCTCCTTCGACGGCGCAATCCTCGGACTGACTTTAGCTGTTAGCGCCGTCGGTTCTGCTCTCAAGTATGCTTCTACCAACGCCGCGT
TGAAGAAGATCAAAGATGCACCCGAAGTCTCCATTTCCGACCTCCGATCACTGCTTCCGGCTTCTGAAGACAAATCAGAGACTAACGATAAC
AGGAAGAGCAATGATCAGCGAATCGTTGTGGTTCGCGGAGTCGTCAAACCGAAAATTTCCGGTGATGAGGGTTACAAGAACAACAACGTGTT
AATCTCTCCGGAGACTGGAGATAAAGCTCTTATCATTCAGAGAACGCAAACTTATGTGTATAGTGGATGGAAGAGATTGTTTCAGTCCACTG
GTCACCGGTTTATGTTGGAGAGATCATTGCGCAAACACGGTGCTGATTTTACGAGAACGGTCCCATTTGTCATTGTTGGAAAGGATCAGCAA
TCGAACTCTAGTTTTGTCGCTGTTAACATGGATGGCTCAAGGCAACCTCTTCCGCTTACCACTGTGTATAATCGCTTGCAGCCTATTAATTC
TTCGTTTCTTCAAGCCTTTTTGTACCCCGATTATCCTGTTGGTCTGCTTGATATAGAGAAGATTTTACCACCTGGGAAAGATATAACAGCTG
TTGGCATCTACAGTTTTAATAATGGAGTTCCTGAAATCAAATCGTGCCAAGATCTTCCTTATTTCTTATCAGAGATGACCAAGGACAAGATG
ATTGAGGACCTTATGGAACAGACCAACTTTATATTCTTGGGCAGCGTTATCTTGGGCATTGTGTCTGTTGGCATCCTTAGCTATGCTGCTGT
CAGGACCTGGAATAAATGGAAACAATGGAACCATCAGAGAGAACTGCCACAGAGACCGAACGATTCTGTGGTCGATGATGAACCCGAAGATG
CAGATGAAATCCCAGATGGAGAATTGTGTGTGATCTGCGTGTCAAGAAGGAGAGTACCTGCGTTTATTCCCTGTGGACATGTAGTATGTTGC
AGGCGATGTGCTTCAACCGTGGAACGAGAGTTAAACCCTAAGTGTCCAGTTTGTCTTCAGAGCATTAGGGGATCTATGCGTGTATATTACTC
TTAGTGACCCTGAATCTTCTCTCATTGTATGTAATAGGGTAATGCAATTTGTACTCTCAGAGAATCGGAAGTTAACAAAATAGTTTGACAAT
GATTTGAAAGTTCGTCAAGTCACTCAATATAGAGTGAGAGATTGTAGAGGATATGAAAGCAAAGTTTTTAAAAGGAAGAAATTTTGTAACCG
ATAACA
AtSPL2 peptide sequence
SEQ ID NO. 9
MSSPERALLNLLTDIALSFDGALLGLTLAVSAVGSALKYASTNAALKKIKDAPEVSISDLRSLLPASEDKSETNDNRKSNDQRIVVVRGVVK
PKISGDEGYKNNNVLISPETGDKALIIQRTQTYVYSGWKRLFQSTGHRFMLERSLRKHGADFTRTVPFVIVGKDQQSNSSFVAVNMDGSRQP
LPLTTVYNRLQPINSSFLQAFLYPDYPVGLLDIEKILPPGKDITAVGIYSFNNGVPEIKSCQDLPYFLSEMTKDKMIEDLMEQTNFIFLGSV
ELGIVSVGILSYAAVRTWNKWKQWNHQRELPQRPNDSVVDDEPEDADEIPDGELCVICVSRRRVPAFIPCGHVVCCRRCASTVERELNPKCP
VCLQSIRGSMRVYYS
solSP1 genomic sequence
SEQ ID NO. 10
ATGGTTCCATGGGCCGGACTCTCTTGCTGTTTGAGTGCAGCTGCTCTTTACCTTCTCGGTAGGAGCAGTGGAAGGTTTGAACTTCTCTTCTT
CCAATTTCATTGCTCTACTGTGTATATTTATGTCCATGAATTTAGATGGATTTGATTTCTAATACAGAGATGCTGAAGTTCTTAAATCCGTT
ACAAGGGTTAATCAATTGAAGGATCTTGGTAAGTAAATTTGTAATTTCATATGGATGGCTTAGTCTCTCCGCTAATTACTTTCAGCAGACGT
TTCAATTACCAGTACAAGGAGGTTGCGTCATATCTTTACTGCATCCATCGTTCATAAGAAAAAGAACTAACTTTTTATCCATTGGGAAAAGA
GCAGATGGATTTTTAGTTTTAGTGTGTGATATCATGCTACACTTGTAACTCATTTGAGGTAGTATGTTCGACCATAGGGAAAATTTCTATTT
TGGCACCAAATGAGAGTCAGGCAGTGTTAGATGATATACTTAGGCGCATCAGTCCCATGGTAAGTTCGAGTAAATTGGGGATACTATTCAAA
GTGGATGGAGGAAAGGTGTGTGACCATGCAAATTGAGTTTCCTCTTTTAACTGGATGGATCTCCTTTTGGTTTCTCTACGAGTAGTAGAGGC
ATTAGACAAGGATATCCTCATCTTCCTGTGTTCCTTTTAGTTATGAAGGTGCTCAGCGAATTAGTGGAGTAGCTGAGAACATGAGGCTGTTC
ATGATATTTGAAAGAGGGATATCTGTTAGATCAGAGGCATTACAAATTCTAGAATATAGAACTTAATATTGGATAGTTGTATATATCTTATT
GTACCTTTTGTTAGTGCATTCTCTCAACCTTGAATTATCTTTGTACTCCTTTTATTTTAGCACAACTACTAGATACTGCATCCAAGGTGTTG
CCTCTGGTGGTTACTATATCTGGAAGGGTTGGATCAGATACACCAATTAACTGTGAGTACAGTGGTCTACGAGGCGTGATTGTGGAAGAAAC
TGTATGTTATCTCGTTCTTTAAATAATAGCATAAAATGTTTGTTTTCTTTTCAATGATGTTTTGTTAAATGTTCTTGATATTATTACAGGCC
GAACAACATTTTCTGAAACACAATGATGCAGGTTCTTGGATACAGGATTCTGCCTTGATGCTCTCAATGTGTAAAGAAGTTCCGTGGTACCT
GGTTGGAATGACTTCCTTTTGTCTTTTTATCTTAATAGTCCTGTAGGCTGTTTAAATGAATTCTTAAGAATTCTGCTTTACTTTGATGTATG
AATTTTTAATAGTTTTTGACTTGTGTACCAGGATGATGGCACAGGTCGCACTTTTATTGTTGGTGGCCGTGGTGCCACGGGTTTGGTACTGA
CAGTTGGAAGTGAAGCCTTCGAGGAAGCAGGGAGATCATTTGTGCGAGGGACATTGGATTATCTTCAAGGTCTTAAGGTCTGTTTTATCATA
TAACTGCTGTTTGTTGCAGGTTGTAATTTGTATTTTCATAACTCTTGTGTCTAAAAAAATTTTGAAGGTAACTTTGACTCTGACCTATTATT
TTCACAAGTAGGAGGCCAACGCCATTTTTAATTAGCGTCGTAAGTGTAGAACATAGTGAATTGCAGGAAACCACTCTCACCAGTCACTCACA
TATCCGTTCATGTATGACCATGCTTTTATGTATTTTGGTGAAGTTCATTAATTGATGATTACTTTTGAAGATGCTTGGAGTAAAGAGGATTG
AACGTGTGCTGCCAGTTGGTACTCCTTTGACTGTTGTTGGCGAGGTATGATATGCCTTTTGAGCAGAAAAAGTTGCAACATTTTCATAGAAG
TAACACAAATTAACAGAATTCAGATGATACGAGTAGCAACTCTCTGTCAGCTTATTCTTTAGTCGTTCACTATGTATTCTCAACACTGATGT
CATAAAAAATGTATTCCTGACACTAACTGTAAATAGAATTGATGACAGCTAAGAAGAATGATAGTGAAACAAAACACCTTCTATATACTTAA
TTAATTCTCAACACCTTCTATATACTTAATTACTTCTCAACACCTTCTATATAGTTCTTTATTTTCTGTAAGGTAACTCTTAGTGTAGTTTC
CAGAGATCTTTGTTGCATCACGTTATTGTGGTTATTGGAAGGAAAGAAGAACAAGAACAATTACAAGAGGTGACTTCCAGAGAGGATTCAAC
AATAGAACAAACTCAAAATGATCCAAGAGTTGCATATATTTAACTAACTGGTTAAATGGTGCACGTCCTTGAATTACATAAAATACATATCA
AGACCTATGGACATCCTCAATATATATAAAATGATTCAGTTGTTTTGTCACAGTCGAGTGGAAGCAAGCATGCAGAGTTTAGACTTTCTTTT
AATGTGTCATTCCTTGTGTTCTTCATGGTCATCATGACCTGATTGGTAGAGAATAGTTTTAAAGGGAAATAATAAAAATAGCAAATAGATTA
TAGAAATAGCTATCAAGGAAATAAGAGGATCAAAATGCATCCAATATTACAAGTATACTTTAGTTATACTTGAGCATTCATTGTTCACTTCT
ACTACATTACTCTGCTTGAACACTAGTTGACCCTTCTCTGTGTGCGCCAAGGGGGCTGGATGCATATAAGAGCTGTCTGTATATTGTGTATC
AACTTCATTTTCCTTTGTGATTGTATCAGGCTGTCAAAGATGACATTGGGACAGTTCGGATCCAGCGACCACACAAAGGCCCATTTTATATC
TCTCATAAAACTATTGACCAGCTCATTGCAAATCTTGGGAGATGGGCAAGGTTGATATGCTGTACTTTCGAACTGTAATCAACCCTATATTC
CCCTACTAATTTTACATATTTTTTCACATGCCCATATCCAATCTTATGTTCAAGCTCATAGACATGAGTACGTTCTACTTCAATTAGGTTGG
TAGTGTACTTTGTAGGGTCTGTATCAAGTACCCTTTCCACTATCATATCTGTTCAACAAAGATATGTCTAGAAAAATAAAAGTTCATGAATT
GACTTGCATGAACATATCTATTCCCCCATGAGTATGTCAAGGCGCCCCCAGGATTTGTTTGTTTTAGCCTCGTACATAGTATTGATAAGTTC
TTTTACTTTCAGGTGGTACAAGTATGCTTCAATGGGTTTTACTGCTGTCGGTGTATATCTACTTGTGAAGCATACTTTTCATTATATGATGG
AAAGAAAGCGTCACTGGGAATTGCGCAGGAGGTATATTTAGGCCAAGACTTCTAAAACTGCAGTCTTTTATCTTTTTTTTTTCTTTTGCCCC
TTACTAATCCTCCCTTCTGAGTTTTGTATCTATCAAAGCACTACTTAAATGTTTGGGTACATTAGAGATGCTTGTACAATTTACTATAGGGT
AGGTGCTGCAACTTGTACCTCAGTATACTTTCTGTTCATGATTGATTTCAAGGATAAGCTGAGATGCAACAGTTTAAGATGTCTCTTCTTTG
CCTGTGTCCCTTGCTCAATTGGGGCATAACATGAGCTGCTGGGAAAAGATGCTTTGGGAGATGTCAAGGTGCCTGCAAGCTGGACTAAGCGC
CACAATTTTTAAAAAAGATATGCTTCAGAGATGGTAAAGCCAGATTTGTAGTAATTTAGAGATGAGGATTTGATGCAGTACTAGAAAGAGGA
CAGAGGGACGAGATAGAGGGTATAAATTAGCAAGGGGAGAAAGAGGGAAAGTTAACAGGAGAGCGAGAGAAAGGACAGAATCTATTACAGGG
AGTTATTTACGAAGTGTTTGTTCATAAACTGCATTAGAGTTTAGTGACAACAAATTAGAAAGTACCTAAAATGACAAAATACTAGGAGCTTT
CAATGCATATAAACAATGGGTGCATATTCCATTTTCATGCACAAAATGTACTTCGTGTGTTAGAAAGGAGGCATAATTTGAAGATGCCATTG
GTAGGGACAGGAAAAATCTTTAGGGGTCGTTTGGTTGGGAATTAGTTATCCCAGGATTAACTATCCCGGGATTAGCTATCCTGGGATAACTT
ATCTCACCATGTATATTGATTAACTTTTCCCATCACTATGATATAAATGGTGGGATGAGTTATTCCCAAACAACATAATTTTTTTCATTTCG
TCACTATTTCCTTATCCCATCACTATTATTTTTAATTCCTCACACCAAACGAGTCCTTAGACTTTACCTATTATCAGGTTCTTACGCATAAT
GCACCCCTCTCAAATAAGAAAGCACCATATTCTGGATACTACTAAAAGTTGAGGAAGACATGTTATACTGAATTGTAATAGAAAATGAGGAA
GATCTTTATATCATGCTCTTTTCCACATGGGAGAGATGGACTTTTTTTCTGAGGGCAGCAACGATGATGAGGTTTGTGCCCAAGAATAGGCT
ATACATGTGTATCTTGATTGGATGTTTTCAGTCCGTTGACTCGCCCTTCCTGAGAATGAATGGAGTAAATTACTATGAACACATATTTTGAT
GAATGCCACTAACACCAAATTGCAGGACCCAATTCTTAGATAAGCAAAGTGTAGCAGGCTAAGGTGAGCGTTAGCCCCTTAAAATATTTGTG
ACCTAGTATTTTTCAGCTTAAGGTAATTTTCTTGGTATTTACCTCCTTCCTACTTTGTGAAGAGCTTGATATTTAGCCTGTATGGCACTTAT
GTGAGTATTTTGGTGGAACAGAATTGGAGAGTTTTTGAAGGGTGGAATATGACTTTGCATTGCTTAGGCTTGCCTTTTATTATTGATTAACT
TTTGGCACACCCACGAGGTCCCTGTTAGTATAGATGAATTATTATCTTATCATAGAGAAGTGTTATTTTATGGGAGTTGTCTACTTTTTGGT
ACACATATTGTCTAACATGTTTTCTCCTCATAACATCAGTAAGCTGTTAATTTTTTATTTTTTTTCGAAAGCTTTGTGTCGGAGGTTCATCA
GTTATGGAGCTTGTTAAACCAAACAAAAAATGTCGGTGATTCTTTTTGATACATATTTGATAACGGTACTGACTACTGACAATGATGAGTAA
CACCAAGAAGGTGCAAGAGCAAACTTACAAAAATGACAAGTGTTGCATTCACATCCATTCTCAAGATAATTGAACTTCATCTCCGCTTTACC
TACAAATATAATTAAATTCCCAGCTAATTACGGTGCCGCCACATAATAGGAAAGATAAAGCAGTATGGCATACTTTGTGCATTTTTTTCATG
TTAGTAGATTTATTACAAATCTAGCACTTTTGTATAAGGTCCTTATAGGGAATGTTATCCTTAGTTTATCCTGGCTAGTATAGGATATAGAC
ATATGACTTGCATATGTATGTCCTTTTATTGGCTTTCTCTCTAGTTTTGGTGAATTCTTGATTCTATTAGTGAGAACTTCATTTTGGCTAAT
ATGATGCTTGTTTTTATGAGGTTGAGATTGATTAGGTTGGTCAATCCAATTAACATCAACTTAAAACAGTCCTGAGTAGTGTAACTTTGCTC
TTCTGTTTTCCCTTTAAAATTTCCAATCCTGCAAGGTTGACTCATCTGGTTTAGGATGTGACCTTGCCAAAAGTTCTGGTGGTCTAGATTTC
TTAACAGAACTCTTTTGGCTTTAAACATCTTGAAATATGGATAGATTGTGTTCCTTCCATTTTTATGTTCATATATTGCTGGATTTCTTCCA
TTGATGTTGTTACAACACCGTACATTTCAACTAACAGTTTTGACTTTTTGGTTTTTCCTTTATTTTACCTTTTTTGAGTGATAATCCTAAAT
TTGAGAAACACTTGCACGTGCAGAGTTCTTGCTGCTGCTGCTAAAAGAGCAGGAGATGAGGATGAAGGTAAGCTTGCTTGGTTTACATACTT
CCACCTTATTGTCTCAACCTTTTGGTTTCTTTGCAGTCAAATTGTTTTGTTTTGCTATCTTTTCTATTTTTAGGTTCTCACTAATCATTTTA
TTTTGTTTCCTTTTCTATTTTTAGGTTCTGATGCTACAGCTGAGAATGGAGTGGATAATAAGGACCTCTTAATGCCAGATCTATGTGTTATA
TGTCTGGAGCAGGAATACAACTCAGTTTTTGTCCCGTAAGACCCTTTCATGAAGGCTTTTAGTTCACTACATGTTCAAATGTTCAGATTTTT
GGGACATTGCTATGCTGCTTAATTGATAAAATTTTAGGAATTGTATGCGAATCCCAACTTGTTTGGTGTTGGCACAATAGAAGTAGAGTTCA
TATAAGTAATACCAACTAACTCAAGATAAAACTACTGTTTTAAGATTAATTAATTCAGATATAACTAGTTTACAACAAAGATCTTGAATGGC
AAATTGTGGAAAGGAGTGTGTAAATAGAGTGGAATGGATATGGAGACTCGTGCAGCTTCTCAAACTATTTTGGATTGAGGGATATCTGAGTT
CATTTGATTTGATTAATGACGGATCAGCTTCCTTTTTGTCCCTGTTTAGGTTGCTGAATGAGATTCTGAAAAAATGAGCATCTCTGAGACTT
GGCATCAGATATTCCTATTTTTGTTTTTGTGTTGGGTTGATCCAAGGGTCTTTCCGAAACAGCCTCTCTATCTTCACAAGGTAGACCTGACT
GGTGGTGTTAGTTGTTGTTTAACATTGTAGTATTGCCCATAAGAGCGGGCTGGCAACACTGTTACTTGGAACAGCAGATAGGATACTGAAGC
TGTAGTGTCCTCACCCTCTAATATAATTAATTTGACAGCCCAGGCGCTAATGCGAAACTTTTGGACTTTTATGTTTGTTTGGTTTTTGTGTA
GAAATTAAAATTTTATTTTGTTTTCGCAGGTGTGGTCATATGTGCTGTTGCATGACGTGCTCTTCACACTTGACAAACTGCCCACTATGTAG
GAGACGGATCGAGCAGGTTGTGAAAACTTTTCGCCATTGA
solSP1 cDNA LOCUS XM_004242278 The underlined sequences represent target sequence for
amRNA1 and amRNA2
SEQ ID NO. 11
actcccaaattattttatggctacaaaaaaaaaaaaaaaaaaaaacgaagaagaagaagaagaagaagtggatggatggattatagagttgt
tgaaggagaggtagataaagagcatgaacataactgcactctcaagtttcctctcaaccactcttagcatcttatggagtgtcagcttcttc
aaattccttttattttttaatcccaatttgtctcttcagttttactgttataaagccacatatgctctgcccttctgtaattgctctatttt
tgatttccatgattgcttgtcgcctgaaacttgtgttgcttagtcataatttcattcgttaggtttaggttctattatcccttcgagctacc
ggaaatggttccatgggccggactctcttgctgtttgagtgcagctgctctttaccttctcggtaggagcagtggaagagatgctgaagttc
ttaaatccgttacaagggttaatcaattgaaggatcttgcacaactactagatactgcatccaaggtgttgcctctggtggttactatatct
ggaagggttggatcagatacaccaattaactgtgagtacagtggtctacgaggcgtgattgtggaagaaactgccgaacaacattttctgaa
acacaatgatgcaggttatggatacaggattctgccttgatgctctcaatgtgtaaagaagttccgtggtacctggatgatggcacaggtcg
cacttttattgttggtggccgtggtgccacgggtttggtactgacagttggaagtgaagccttcgaggaagcagggagatcatttgtgcgag
ggacattggattatcttcaaggtcttaagatgcttggagtaaagaggattgaacgtgtgctgccagttggtactcctttgactgttgttggc
gaggctgtcaaagatgacattgggacagttcggatccagcgaccacacaaaggcccattttatatctctcataaaactattgaccagctcat
tgcaaatcttgggagatgggcaaggtggtacaagtatgcttcaatgggttttactgctgtcggtgtatatctacttgtgaagcatacttttc
attatatgatggaaagaaagcgtcactgggaattgcgcaggagagttcttgctgctgctgctaaaagagcaggagatgaggatgaaggttct
gatgctacagctgagaatggagtggataataaggacctcttaatgccagatctatgtgttatatgtctggagcaggaatacaactcagtttt
tgtcccgtgtggtcatatgtgctgttgcatgacgtgctcttcacacttgacaaactgcccactatgtaggagacggatcgagcaggttgtga
aaacttttcgccattgagcagaaagctgccttgttgaaatggccattttctgactcgctcataaaaatcattcggatattaacttgtagatt
gtattgaaagctggattttgtcagagttttatgtcattatgatgtatgtataatagcctctctccatctcaacggttaaactagca
solSP1 peptide
SEQ ID NO. 31
MVPWAGLSCCLSAAALYLLGRSSGRDAEVLKSVTRVNQLKDLAQLLDTASKVLPLVVTISGRVGSDTPINCEYSGLRGVIVEETAEQHFLKH
NDAGSWIQDSALMLSMCKEVPWYLDDGTGRTFIVGGRGATGLVLTVGSEAFEEAGRSFVRGTLDYLQGLKMLGVKRIERVLPVGTPLTVVGE
AVKDDIGTVRIQRPHKGPFYISHKTIDQLIANLGRWARWYKYASMGFTAVGVYLLVKHTFHYMMERKRHWELRRRVLAAAAKRAGDEDEGSD
ATAENGVDNKDLLMPDLCVICLEQEYNSVFVPCGHMCCCMTCSSHLTNCPLCRRRIEQVVKTFRH
B. rapa SP1
SEQ ID NO. 32
MIHWGGVTCCLSAAALYLLGSSSGRDAEVLKTVTRVNQLKELAQLLELDSSKLLPFIVAVSGRVGSDTPIKCEHSGIRGVIVEETAEQHFLK
HNETGSWVQDSALMLSMSKEVPWFLDDGTSRVNVVGARGATGFALTVGSEVFEESGRSLVRGTLDYLQGLKMLGVKRIERVLPTGMPLTIVG
EAVKDDIGDLRIQKPERGPFYVSPKSLDQLISNLGKWSRWYKYASMGLTVFGVFLITKHVIDHVLERRRHRELQKRVLDAAAKRAEETEGSN
GAHESVSDSTKKEGAVPDLCVICLEHNYNAVFVPCGHMCCCTACSSHLTSCPLCRRRIDQVVKTYRH
C. papaya SP1
SEQ ID NO. 33
MIPWGGITCCMSAAALYLLGRSSGRDADILRKVIRVNQLKELAQLLDIESKIMPLIVAISGRVGSETPISCEYSGLRGVIVEETAEQHFLIG
INDAGSWIQDSALMLSMSKEVPWYLDDGTARVFVVGARGATGFVLTVGSEVFEESGRSLVRGTLDYLQGLKMLGVKRIERVLPTGTSLTVVG
EMTLEQFRYEDRTNGLFTCLQINGSPDFQLGKWARWYKYASMGLTFLGVFLIAKRSIEYVLERRRRRELQKRVLAAAAKRSGQDNEGSNIIP
ENGLDGAKREHLMPDLCVICLEQEYNAAFVPCGHMCCCMTCSSHLTNCPLCRRRIEQVLRTFRH
C. sinensis SP1
SEQ ID NO. 34
MIPWGGISCCLSGAALYLLGRSSGRDAELLKTVTRVNQLKELAHLLDSGSKVLPFIVTVCGRVGSETPISCEYSGLRGVIVEETAEQHFLKH
NDAGSWIQDSALMLSMSKEVPWYLDDGTGRAFVVGARGATGFVLTVGSEVFEESGRSLVRGTLDYLQGLKMLGVKRIERLLPTGTSLTVVGE
AVKDDIGTVRIQRPHKGPFYVSPKTIDELIENLGKWARWYKYASFGLTIFGTFLIAKRAIHYILQRKRRWELHRRVLAAAAVKRSEQDNEGT
NGQAENGSDGTQRDRVMPDLCVICLEQEYNAVFVPCGHMCCCIICSWHLTNCPLCRRRIDQVVRTFRH
C. clementina SP1
SEQ ID NO. 35
MIPWGGISCCLSGAALYLLGRSSGRDAELLKTVTRVNQLKELAHLLDSGSKVLPFIVTVCGRVGSETPISCEYSGLRGVIVEETAEQHFLKH
NDAGSWIQDSALMLSMSKEVPWYLDDGTGRAFVVGARGATGFVLTVGSEVFEESGRSLVRGTLDYLQGLKMLGVKRIERLLPTGTSLTVVGE
AVKDDIGTVRIQRPHKGPFYVSPKTIDELIENLGKWARWYKYASFGLTIFGTFLIAKRAIHYILQRKRRWELHRRVLAAAAVKRSEQDNEGT
NGQAENGSDGTQRDRVMPDLCVICLEQEYNAVFVPCGHMCCCIICSSHLTNCPLCRRRIDQVVRTFRH
C. sativus SP1
SEQ ID NO. 36
MLPWGGISCCLSAAALYLLGRSSGRDAELLKSVTRVNQLKELAQLLEAEHLLPLVVAISGRVSSDTPINCEFSGLRGVIVEETAEQHFLKHN
DAGSWIQDSALMLSMSKEVPWYLDDGTGRAFVLGARNATNFELPVVSEVFEESGRSLMRGTLDYLQGLKMLGVKRIERVLPTGTSLTVVGEA
AKDDIGTIRIQRPHKGPFYVSPKTIDQLISNLGKWARWYKYASMGLSIFGLYLVTKHVILYLMERRRRWELQKRVLAAAAKRSSQENEGEIE
KASNGTDGTKRDRSMPDLCVICLERDYNAVFVPCGHMCCCVACCSHLTNCPLCRRRIELVVKTFRH
P. persica SP1
SEQ ID NO. 37
MLPWGGLSCCLSAAALYLLGRSSGRDADILKSATRINQLKELAKLLDSECILPLVVAISGRVSSETPITCEFTGLRGVVVEETAEQHFLKHN
DAGSWIQDSALMLSMSKEVPWYLDDGTGRVHVVGARGATGFVLPVASEVFEESGRSLVRGTLDYLQGLKMLGVKRIERVLPTGTSLSVVGEA
VKDDIGTIRIQRPHKGPFYVSPKTIDQLIANLGKWARWYKYASLGLTVFGVYLVAKHSVQYILERRRRWELQRRVLAAAAKRSGEDNEGSNE
KDDNVLDGSKRLMPDLCVICLEHEYNAVFVPCGHMCCCTTCSLHLTNCPLCRRRIDQAVKTFRH
F. vesca SP1
SEQ ID NO. 38
MLPWGGLSCCLSAAALYLLGRSSGRDADILKTATRVSQLKELAKLLDSESILPLVVAISGRVSSETPITCEFSGLRGVVVEETAEQHFLKHN
DAGSWIQDSALMLSMSKEVPWYLDDGTGRVHVVGARGATGFTLPVASEVFEESGRSLVRGTLDYLQGLKMLGVKRIERVLPTGTSLSVVGEA
VKDDIGTIRIQRPHKGPFYISPKTIDQLIANLGKWARLYKYASLGLGVFGVYLIAKHSVHYIMERRRRWELQKRVLAAAAKRSGEDIEGSND
IEYNASEAPKKDRLMPDLCVICLEQEYNAVFVPCGHMCCCTTCSLHLTNCPLCRRRIEQVVKTFRH
G. max SP1 A
SEQ ID NO. 39
MIPWGGLSCCLSAAALYLLGRSSGRDAELLKSVTRVNQLKELAQLLDAEILPLIVTISGRVSSETPINCEFSGLRGVIVEETAEQHFLKHND
AGSWIQDSALMLSMSKEVPWYLDDGTDRVHVVGARGAAGFALPVGSEAFEESGRSLVRGTLDYLQGLKMLGVKRIERVLPVGTSLTVVGEAA
KDDVGAFRIQRPBKGPFYVSPKITDQLIANLGKWARWYKYASMGLTVFGAYLIAKHAIRYILERRRRSELQRRVLAAAAKKSGQNNDVEKAD
GLSDGVKKDRLMPDLCVICLEQEYNAVFVPCGHMCCCTTCSSHLTNCPLCRRQIEKVVKTFRH
G. max SP1 B
SEQ ID NO. 40
MIPWGGLSCCLSAAALYLLGRSSGRDAEILKSVTRVNQLKELAQLLDAEILPLIVTISGRVSSETPINCEFSGLRGVIVEETAEQHFLKHND
AGSWIQDSALMLSMSKEVPWYLDDGTDRVHVVGARGASGFALPVGIEAFEESGRSLVRGTLDYLQGLKMLGVKRIERVLPVGTSLTVVGEAA
KDDVGAIRIQRPHKGPFYVSPKTIDQLIANLGKWARWYKYASVGLTVFGAYLIAKHAIRYILERRRRSELQRRVLAAAAKKSGQNNDVEKAD
SLSDGAKKDRLMPDLCVICLEQEYNAVFVPCGHMCCCTACSSHLTNCPLCRRQIEKVVKTFRH
P. vulgaris SP1
SEQ ID NO. 41
MKMIPWGGLSCCLSAAALYLLGRSSGRDAEILKSVTRVNQLKELAQLLDAEILPLIVTISGRVGSETPISCDFSGLRGVIVEETAEQHFLKH
NDAGSWIQDSALMLSMSKEVPWYLDDGSDRVHVVGARGATGFALPVGSEAFEESGRSLVRGTLDYLQGLKMLGVKRIERVLPVGTSLTVVGE
AAKDDVGTIRIQRPHKGPFYVSPKTIDQLIANLGKWARWYKYASMGLTVFGAYLIAKHAIRFILERRRRSELQRRVLAAAAAKKSGPNNDVE
KADSLSDVAKRDHLMPDLCVICLEQEYNAVFVPCGHMCCCTACSSHLTNCPLCRRQIEKVVKTFRH
M. truncatula SP1
SEQ ID NO. 42
MAMVPWRGVGCCLSAAALYLLGRTSGYVDILKSVNRVNQLRELAQLLDEEIFPLVVAISGRVGSETPISCEFSGLRGVIIEETAEQHFLKHS
DAGSWIQDSALMQSRSNEVPWYLDDGTGRVRVVGAQGATGFVLPVGSEAFEESGRLPVRGTSDYVQGLKVGVLMLGVKRIERVLPVGTSLTV
VGEAAKDDVGTIRIQRPSKGPFYVSPKTIDELIANIGRWARWYKYASAGLTVLSVYMIANHAVRYILERRRRNELEKRVLAAAAKISGQDNG
GEMDDSLSDGAKRERAMPNLCVICLEQEYNSVFVPCGHMCCCTACSSHLTSCPLCRRQIEKAVKTFRH
P. trichocarpa SP1
SEQ ID NO. 43
MMIPWGGISCCLSGAALYLLGRSSGRDAEVLKSVAKVNQLKELAKLLDIESKVLPLVVAISGRVGAESPISCEFSGLRGVIVEETAEQHFLK
HNDAGSWIQDSALMLSMSKEVPWYLDDGTDRVYVVGARGASGFVLTVGSEVFEESGRSLVRGTLDYLQGLKMLGVKRIERVLPTGTSLTVVG
EAVKDDIGTVRIQRPHKGPFYVSPKSIDELIGNLGKWARWYKYASLGLTVFGAFLITKHVIRYIMERRRRWELQSRVLAAAKRSGQDNEGSN
DKAENGSDGAKRERPIPDLCVICLEQEYNAVFLPCGHMCCCITCCSQLSNCPLCRRRIEQVVKTFRH
V. vinifera SP1
SEQ ID NO. 44
MIPWGGISCCLSAAALYLLGRSSGRDAEALKSVTRVQQLKDLVQLLDTACKVLPLVVTVSGRVGSDTPIKCEYSGLRGVIVEETAEQHFLKH
NDAGSWIQDSALMLSMSKEVPWYLDDDTGRAYIVGARGATGLVLTVGSEVFEESGRSLVRGTLDYLQGLKMLGVKRIERVLPTGTPLTVVGE
AIKDDVGTIRIQRPHKGPFYVSPKSIDHLVANLGKWARWYRYASLGFTVFGVYLIAKSAIQYVMERKRCWELRKRVLAAASKKSGQDSEDPD
EKDENGSDNTKRDRLMPDLCVICLEQEYNAVFVPCGHMCCCTMCSSQLTNCPLCRRRIEQVVRTFRH
S. lycopersicum SP1
SEQ ID NO. 45
MVPWAGLSCCLSAAALYLLGRSSGRDAEVLKSVTRVNQLKDLAQLLDTASKVLPLVVTISGRVGSDTPINCEYSGLRGVIVEETAEQHFLKH
NDAGSWIQDSALMLSMCKEVPWYLDDGTGRTFIVGGRGATGLVLTVGSEAFEEAGRSFVRGTLDYLQGLKMLGVKREERVLPVGTPLTVVGE
AVKDDIGTVRIQRPHKGPFYISHKTIDQLIANLGRWARWYKYASMGFTAVGVYLLVKHTFHYMMERKRHWELRRRVLAAAAKRAGDEDEGSD
ATAENGVDNKDLLMPDLCVICLEQEYNSVFVPCGHMCCCMTCSSHLTNCPLCRRRIEQVVKTFRH
S. tuberosum SP1
SEQ ID NO. 46
MVPWGGLSCCLSAAALYLLGRSSGRDAEVLKSVTRVNQLKDLAQLLDTASKVLPLVVTISGRVGSDTPINCEYSGLRGVIVEETAEQHFLKH
NDAGSWIQDSALMLSMCKEVPWYLDDDTGRTFVVGGRGATGLVLTVGSEIFEEAGRSFVRGTLDYLQGLKMLGVKRIERVLPVGTPLTVVGE
AVKDDIGTVRIQRPHKGPFYISHKTIDQLIANLGRWARWYKYASMGFTAVGVYLLVKHTFHYMMERKRHWELRRRVLAAAAKRAGHEDEGSN
ATAENGVDNKKDLLMPNLCVICLEQEYNSVFVPCGHMCCCMTCSSHLTNCPLCRRRIEQVVKTFRH
S. bicolor SP1
SEQ ID NO. 47
MMIPWGGVGCCLSAAALYLLGRSSGRDAEVLRSVARAGSMKDLAAILDTASKVLPLVVAVSGRVSSDTPLICQQSGMRGVIVEETAEQHFLK
HNDAGSWIQDSAVMLSVSKEVPWYLDDGTGRVYVVGARSAAGLILTVASEVFEESGRTLVRGTLDYLQGLKMLGVKRTERVLPTGTSLTVVG
EAIRDDVGTIRIQRPHKGPFYVSPKSIDQLIMNLGKWAKLYRLASMGFATFGVFLLAKRAIQHFLERKRRHELQKRVLNAAAQRQAREAEGS
NGSSDTEPNSKKDQLVLDICVICLEQEYNAVFVPCGHMCCCMACSSHLTNCPLCRRRIDQAVRTFRH
Z. mays SP1 A
SEQ ID NO. 48
MMIPWGGVGCCLSAAALYLLGRSSGRDAEVLRSVARAGSMKDLAAILDTASKVLPLVVAVSGRVGSDTPLICQQSGMRGVIVEETAEQHFLK
HNDAGSWIQDSAVMLSVSKEVPWYLDDGTGRVYMVGARSAAGLILTVASEVFEESGRTLVRGTLDYLQGLKMLGVKRTERVLPTGISLTVVG
EALKDDVGTIRIQRPHKGPFYVSPKSIDQLIMNLGKWAKLYRLASMGFATFGAFLLAKRAIQHFLERKRRHELQKRVLNAAAQRQAREAEGS
IGSSDTEPNSKKDQLVLDICVICLEQEYNAVFVPCGHMCCCMACSSHLTNCPLCRRRIDQAVRTFRH
Z. mays SP1 B
SEQ ID NO. 49
MMIPWGGVGCCLSAAALYLLGRSSGSDAEVLRSVARAGSMKDLAAILDTASKVLPLVVAISGRVGSDTPLICQQSGMRGVIVEETAEQHFLK
HNDAGSWIQDSAVMLSVSKEVPWYLDDGTGRVYVVGARSAAGLILTVASEVFEESGRTLVRGTLDYLQGLICMLGVKRTERVLPTGTSLTVV
GEAIKDDVGTIRIQRPHKGPFYASSKSIDQLIVNLGKWAKLYRIASMGFATFGVFLLAKRALQHFLERRRRHELQKRVLNAAAQRQAREAEG
SKGTSDAEPNSKKDQLVLDICVICLEQEYNAVFVPCGHMCCCVACSSHLTNCPLCRRRIDQAVRTFRH
S. italica SP1
SEQ ID NO. 50
MMIPWGGVGCCLSAAALYLLGRSSGRDAEVLRSVARAGSMKDLAAILDTASKVLPLVVAVSGRVGSDTPLICQQSGMRGVIVEETAEQHFLK
HNDAGSWIQDSAVMLSVSKEVPWYLDDGTGRVYVVGARAAAGLILTVASEVFEESGRTLVRGTLDYLQGLKMLGVKRTERVLPTGTSLTVVG
EAIKDDVGTIRIQRPHKGPFYVSPKSIDQLILNLGKWAKLYRLASMGFATFSVFLLAKRAIQHFLERKRRHELQKRVLNAAAQRQAREAEGG
KGTSNTEPNSKKDQLVLDICVICLEQEYNAVFVPCGIAMCCCMACSSHLTNCPLCRRRIDQAVRTFRH
P. virgatum SP1
SEQ ID NO. 51
MMIPWGGVGCCLSAAALYLLGRSSGRDAEVLRSVARAGSMKDLAAILDTASKVLPLVVAVSGRVGSDTPLICQQSGMRGVIVEETAEQHFLK
HNDAGSWIQDSAVMLSVSKEVPWYLDDGTGRVYVVGARAAAGLILTVASEVFEESGRTLVRGTLDYLQGLKMLGVKRTERVLPTGTSLTVVG
EAIKDDVGTIRIQRPHKGPFYVSPKSIDQLILNLGKWAKLYRLASMGFATFGVFLLAKRAIQHFLERKRRHELQKRVFNAAAQRQARESEGG
NGTSDTEPNSKKDQLVLDICVICLEQEYNAVFVPCGHMCCCMACSSHLTNCPLCRRRIDQAVRTFRH
O. sativa SP1
SEQ ID NO. 52
MLIPWGGVGCCLSAAALYLLGRSSGRDAEVLRSVARAGSTKDLAAILDTASKVLPLVVAVSGRVGSDTPLICQQSGMRGVIVEETAEQHFLK
HNDAGSWIQDSAVMLSVSKEVPWYLDDGTGRVFVVGARGAAGLVLTVASEVFEESGRTLVRGTLDYLQGLKMLGVKRTERVLPTGTSLTVVG
EALKDDVGITRIQRPHKGPFYVSPKSIDQLIMNLGKWAKLYQLASMGFAAFGVFLLAKRALQHFLERKRRHELQKRVHAAAAQRQAREAEGG
NGTSDVDSNNKKDQLVLDICVICLEQEYNAVFVPCGHMCCCMNCSSHLTNCPLCRRRIDQAVRTFRH
B. distachyon SP1
SEQ ID NO. 53
MMVPWGGVGCCLSAAALYLLGRSSGRDAEVLRSVTRTGSLKDLAAILDTASKVLPLVVAVSGRVSSDTPLICQQSGMRGVIVEEMAEQHFLK
HNDAGSWIQDSAVMLSVSKEVPWYLDDGTGRVYVVGARAAAGLVLTIASEVFEESGRTLVRGTLDYLQGLKMLGVKRTERVLPTGTSLTVVG
EAIKDDVGTIRIQRPHKGPFYASPKSIDQLILNLGKWAKLYQLASMGFAAFGVFLLAKRALQHFLQKKRQHELNKRVRAAAAQRQAREAEGA
DGTSNGDPNSKKDQLVLEICVICLEQEYNAVFVPCGHMCCCMNCSSHVTNCPLCRRRIDQAVRTFRH
AtSPL1 (NP_564745)
SEQ ID NO. 54
MIHLAGFTCCLGGVALYLLTRSTGRDIKSITRVYQLKDLEQLVEVESKVVPLIIAVSGDVGSETPIKCEHSYVLGVFIKRTAEQQVLRRNWR
FSWVRNSTLMQPMTKEVPWYLDDGTGRVNVDVSQGELGLALTVGSDVFEKAEPVSLVQGALGYLKGFKILGVRHVERVVPIGTPLTVVGEAV
RDGMGNVRIQKPEQGPFYVTYIPLDQLISKLGDLSRRFKYASMGLTVLGVILISKPVIEYILKRIEDTLERRRRQFALKRVVDAAARRAKPV
TGGGTSRDGDTPDLCVVCLDQKYNTAFVECGHMCCCTPCSLQLRTCPLCRERIQQVLKIYRH
B. rapa SPL1
SEQ ID NO. 55
MEPWSGLCCIGAVALYLLSRRTARDVDFLKSVTRVDQLKDLESAVLPSIVAVSGTVGSETPIKCEHSGILSVILQETAEQQFLKRNWKFSWV
QDTALMLPVCKEVPWFLDDGTGRVIVEGARSGIGFELTVGGEVFEKPEASSLVRGTLDFLRGLEMLGIRRIERVLPVGTRLTVVGQTIKDGV
GDVRIQKPDQGPFYVSPIPLDHLISKVGKWSRRFKKASMGLAVVGVILISKPVIKYILVRTGDFLERRQQRLLKKRVVDAAAKRKKLAAPKR
EERVTSKGLENGKSRDGDEHDRCVVCLERKCDAAFVPCGHMCCCLTCALKLLGKPCPLCRKRGIRILKIYRN
Z. mays SPL2
SEQ ID NO. 56
MSARDRETAEALVRLAASLDGAVLGLGTAAVAVASWVKYLAVSGQLRLVASAATASIADARSLLSGDGAEPRIAAVRGYVRAHDKFFRAPFS
GEAGVVTKHTQMCLFTEWRGIFGWTFDLHALLFRSWKEQIVTSFRSVPFVLVSTELGNPTGVVHINVDKADQPLPLTTVFHKLIPLETTPYT
LFQTIIGNGYPIALLDEEKILPIGKKITAIGLCQAKDAESVEITSCPEIPFFLSELTKDEMQAQLASRARILFWGSIVLGTLSVCLVGHAIY
RGWTRIKLRREARHARQMFEEAEDAIHRDDSSDDDEIGDGQLCVVCLRKRRRAAFIPCGHLVCCSECALTIERTPHPLCPMCRQDIRYMMRV
YDS

The invention is further described by the following numbered paragraphs:

1. A transgenic plant cell, plant or a part thereof characterised in that
a) the expression of a nucleic acid comprising SEQ ID NO. 1 or 2 and encoding a SP1 peptide comprising SEQ ID NO. 3, a functional homologue or variant thereof is altered or
b) the activity of a SP1 peptide is altered and said plant expresses a nucleic acid comprising a mutant SEQ ID NO. 1 or 2 nucleic acid encoding a mutant SP1 peptide, a functional homologue or variant thereof which carries a mutation in the RING domain.

2. A transgenic plant cell, plant or a part thereof according to paragraph 1 wherein said plant expresses a nucleic acid construct comprising SEQ ID NO. 1 or 2 and encoding a SP1 peptide comprising SEQ ID NO. 3, a functional homologue or variant thereof.

3. A transgenic plant cell, plant or a part thereof according to paragraph 2 wherein plastid development is accelerated.

4. A transgenic plant cell, plant or a part thereof according to paragraph 2 or 3 wherein said construct further comprises a regulatory sequence.

5. A transgenic plant cell, plant or a part thereof according to paragraph 4 wherein said regulatory sequence is a constitutive promoter, a strong promoter, an inducible promoter, a stress-inducible promoter or a tissue-specific promoter.

6. A transgenic plant cell, plant or a part thereof according to paragraph 6 wherein said regulatory sequence is the CaMV35S promoter.

7. A transgenic plant cell, plant or a part thereof according to paragraph 6 wherein said regulatory sequence is a tissue-specific promoter.

8. A transgenic plant cell, plant or a part thereof according to paragraph 7 wherein said promoter is a seed or seedling-specific promoter.

9. A transgenic plant cell, plant or a part thereof according to any of paragraphs 2 to 8 wherein said plant is a seedling and the transition from etioplasts to chloroplasts is accelerated or wherein said plant part is a seed and the transition to amyloplasts is accelerated.

10. A transgenic plant cell, plant or a part thereof according to paragraph 1 wherein the expression of a nucleic acid comprising SEQ ID NO. 1 or 2 and encoding a SP1 peptide comprising SEQ ID NO. 3, a functional homologue or variant thereof is inactivated, repressed or down-regulated.

11. A transgenic plant cell, plant or a part thereof according to paragraph 10 wherein plastid development is delayed.

12. A transgenic plant cell, plant or a part thereof according to a paragraph 10 or 11 wherein expression of the endogenous SP1 nucleic acid comprising SEQ ID NO. 1 or 2 encoding a SP1 peptide comprising SEQ ID NO. 3, a functional homologue or variant thereof is silenced.

13. A transgenic plant cell, plant or a part thereof according to paragraph 12 wherein the transition from chloroplast to gerontoplast is delayed.

14. A transgenic plant cell, plant or a part thereof according to any of paragraphs 10 to 13 wherein said plant part is green tissue.

15. A transgenic plant cell, plant or a part thereof according to paragraph 1 characterised in that the activity of a SP1 peptide is altered and said plant expresses a nucleic acid comprising a mutant SEQ ID NO. 1 or 2 and encoding a mutant SP1 peptide, a functional homologue or variant thereof which carries a mutation in the RING domain.

16. A transgenic plant cell, plant or a part thereof according to paragraph 15 wherein the RING domain is as defined in SEQ ID NO. 4 or a domain that has at least 95 homology to SEQ ID NO. 4.

17. A transgenic plant cell, plant or a part thereof according to paragraph 15 or 16 wherein said mutation is a substitution, deletion or insertion.

18. A transgenic plant cell, plant or a part thereof according to paragraph 17 wherein said mutation is a substitution or deletion of a conserved C or H residue in the RING domain.

19. A transgenic plant cell, plant or a part thereof according to paragraph 18 wherein said mutation is at a position corresponding to C330 in A. thaliana SP1 or at an equivalent position in an SP1 homologue.

20. A transgenic plant cell, plant or a part thereof according to any of paragraphs 1 to 19 wherein said functional homologue has at least 70%, 71%, 72%, 73%, 74%, 75% 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the amino acid represented by SEQ ID NO:3.

21. A transgenic plant cell, plant or a part thereof according to any of paragraphs 1 to 19 wherein said functional homologue is as shown in SEQ ID No. 9, 31, 32, 33, 34, 35, 36, 37, 38, 39, 40, 41, 42, 43, 44, 45, 46, 47, 48, 49, 50, 51, 52, 53, 54, 55 or 56.

22. A transgenic plant cell, plant or a part thereof according to a preceding paragraph wherein said functional homologue comprises a RING domain as defined in SEQ ID NO. 4 or a domain with at least 95% overall sequence identity thereto.

23. A transgenic plant cell, plant or a part thereof according to a preceding paragraph wherein said homologue is SP1 from maize, rice, wheat, oilseed rape, sorghum, soybean, potato, tomato, grape, barley, pea, bean, field bean, lettuce, cotton, sugar cane, sugar beet, broccoli or other vegetable brassicas or poplar.

24. A plant according to a preceding paragraph wherein said plant is a monocot or dicot plant.

25. A plant according to a preceding paragraph wherein said plant is a crop plant or biofuel plant.

26. A plant according to paragraph 25 wherein said crop plant is selected from maize, rice, wheat, oilseed rape, sorghum, soybean, potato, tomato, grape, barley, pea, bean, field bean, lettuce, cotton, sugar cane, sugar beet, broccoli or other vegetable brassicas or poplar.

27. A product derived from a plant as defined in any of paragraphs 1 to 26 or from a part thereof.

28. A vector comprising a SP1 nucleic acid as defined in SEQ ID NO. 1, 2, 7, 8, 10 or 11, a functional variant or homologue thereof.

29. A vector according to paragraph 27 or 28 further comprising a regulatory sequence which directs expression of the nucleic acid.

30. A host cell comprising a vector according to any of paragraphs 28 to 29.

31. A method for altering plastid development in a transgenic plant cell, plant or a part thereof comprising
a) altering the expression of a nucleic acid comprising SEQ ID NO. 1 or 2 and encoding a SP1 peptide comprising SEQ ID NO. 3, a functional homologue or variant thereof or
b) altering the activity of a SP1 peptide comprising SEQ ID NO. 3 comprising expressing in a plant a nucleic acid comprising a mutant SEQ ID NO. 1 or 2 and encoding a mutant SP1 peptide, a functional homologue or variant thereof which carries a mutation in the RING domain.

32. A method according to paragraph 31 comprising introducing and expressing in a plant a nucleic acid construct comprising SEQ ID NO. 1 or 2 and encoding a SP1 peptide comprising SEQ ID NO. 3, a functional homologue or variant thereof.

33. A method according to paragraph 32 wherein chloroplast and/or amyloplast development is accelerated.

34. A method according to paragraph 32 or 33 wherein said construct further comprises a regulatory sequence.

35. A method according to paragraph 34 wherein said regulatory sequence is a constitutive promoter, a strong promoter, an inducible promoter, a stress-inducible promoter or a tissue-specific promoter.

36. A method according to paragraph 35 wherein said regulatory sequence is a tissue-specific promoter.

37. A method according to paragraph 36 wherein said promoter is a seed or seedling-specific promoter.

38. A method according to any of paragraphs 33, 34 or 35 wherein said plant is a seedling and the transition from etioplasts to chloroplasts is accelerated.

39. A method according to paragraph 32 comprising inactivating, repressing or down-regulating the expression of a nucleic acid comprising SEQ ID NO. 1 or 2 and encoding a SP1 peptide comprising SEQ ID NO. 3, a functional homologue or variant thereof.

40. A method according to paragraph 39 wherein plastid development is delayed.

41. A method according to a paragraph 39 or 40 wherein expression of the endogenous SP1 nucleic acid comprising SEQ ID NO. 1 or 2 and encoding a SP1 peptide comprising SEQ ID NO. 3, a functional homologue or variant thereof is silenced.

42. A method according to paragraph 41 wherein the transition from chloroplast to gerontoplast is delayed.

43. A method according to any of paragraphs 39 to 42 wherein said plant part is green tissue.

44. A method according to paragraph 31 comprising altering the activity of a SP1 peptide comprising SEQ ID NO. 3, a functional homologue or variant thereof is altered comprising expressing in a plant a nucleic acid comprising a mutant SEQ ID NO. 1 or 2 and encoding a mutant SP1 peptide, a functional homologue or variant thereof which carries a mutation in the RING domain.

45. A method according to paragraph 44 introducing and expressing in a plant a nucleic acid comprising a mutant SEQ ID NO. 1 or 2 and encoding a mutant SP1 peptide, a functional homologue or variant thereof which carries a mutation in the RING domain.

46. A method according to any of paragraphs 44 to 45 wherein the RING domain is as defined in SEQ ID NO. 4 or a domain with at least 95% overall sequence identity thereto.

47. A method according to any of paragraphs 44 to 46 wherein said mutation is a substitution, deletion or insertion.

48. A method according to paragraph 47 wherein said mutation is a substitution or deletion of a conserved C or H residue in the RING domain.

49. A method according to paragraph 47 wherein said mutation is at a position corresponding to C330 in A. thaliana SP1 or at an equivalent position in an SP1 homologue.

50. A method for delaying green tissue senescence comprising inactivating, repressing or down-regulating the expression of a nucleic acid comprising SEQ ID NO. 1 and encoding a SP1 peptide comprising SEQ ID NO. 3, a functional homolog or variant thereof.

51. A method for increasing grain/seed size and/or starch content comprising introducing and expressing in a plant a nucleic acid construct comprising SEQ ID NO. 1 or 2 and encoding a SP1 peptide comprising SEQ ID NO. 3, a functional homologue or variant thereof.

52. A method for increasing yield of a plant comprising increasing, inactivating, repressing or down-regulating the expression of a nucleic acid comprising SEQ ID NO. 1 or 2 and encoding a SP1 peptide comprising SEQ ID NO. 3, a functional homologue or variant thereof or introducing and expressing in a plant a nucleic acid comprising a mutant SEQ ID NO. 1 or 2 and encoding a mutant SP1 peptide, a functional homologue or variant thereof which carries a mutation in the RING domain.

53. A method for improving seedling emergence, growth and survival comprising introducing and expressing in a plant a nucleic acid construct comprising SEQ ID NO. 1 or 2 and encoding a SP1 peptide comprising SEQ ID NO. 3, a functional homologue or variant thereof.

54. A method for altering fruit ripening in a transgenic plant cell, plant or a part thereof comprising
a) altering the expression of a nucleic acid comprising SEQ ID NO. 1 or 2 and encoding a SP1 peptide comprising SEQ ID NO. 3, a functional homologue or variant thereof or
b) altering the activity of a SP1 peptide comprising SEQ ID NO. 3, a functional homologue or variant thereof.

55. A method for making a transgenic plant with altered plastid development comprising
a) altering the expression of a nucleic acid comprising SEQ ID NO. 1 or 2 and encoding a SP1 peptide comprising SEQ ID NO. 2, a functional homologue or variant thereof or
b) altering the activity of a SP1 peptide comprising SEQ ID NO. 2, a functional homologue or variant thereof.

56. The use of
a) a nucleic acid comprising SEQ ID NO. 1 or 2 and encoding a SP1 peptide comprising SEQ ID NO. 3, a functional homologue or variant thereof or
b) a siRNA or miRNA targeting SEQ ID NO. 1 or 2 and encoding a SP1 peptide comprising SEQ ID NO. 3, a functional homologue or variant thereof or
c) a nucleic acid encoding a mutant SP1 protein
in altering plastid development, increasing yield, increasing seedling survival, increasing growth and/or emergence, increasing seed/grain size, delaying green tissue senescence or delaying fruit ripening.

57. A method for increasing stress tolerance to one or more of salinity, osmotic stress and/or oxidative stress in a plant cell, plant or part thereof comprising introducing and expressing in said plant cell, plant or part thereof a nucleic acid construct, comprising a nucleic acid comprising SEQ ID NO. 1 or 2 and encoding a SP1 peptide comprising SEQ ID NO. 3, a functional homologue or variant thereof.

58. The use of a nucleic acid construct comprising a nucleic acid comprising SEQ ID NO. 1 or 2 and encoding a SP1 peptide comprising SEQ ID NO. 3, a functional homologue or variant thereof in improving stress tolerance to one or more of salinity, osmotic stress and/or oxidative stress.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A transgenic plant cell, plant or a part thereof characterised in that
a) the expression of a nucleic acid comprising SEQ ID NO. 1 or 2 and encoding a SP1 peptide comprising SEQ ID NO. 3, a functional homologue or variant thereof is altered or
b) the activity of a SP1 peptide is altered and said plant expresses a nucleic acid comprising a mutant SEQ ID NO. 1 or 2 nucleic acid encoding a mutant SP1 peptide, a functional homologue or variant thereof which carries a mutation in the RING domain.

2. A transgenic plant cell, plant or a part thereof according to claim 1 wherein said plant expresses a nucleic acid construct comprising SEQ ID NO. 1 or 2 and encoding a SP1 peptide comprising SEQ ID NO. 3, a functional homologue or variant thereof.

3. A transgenic plant cell, plant or a part thereof according to claim 2 wherein plastid development is accelerated.

4. A transgenic plant cell, plant or a part thereof according to claim 2 wherein said construct further comprises a regulatory sequence.

5. A transgenic plant cell, plant or a part thereof according to claim 4 wherein said regulatory sequence is a constitutive promoter, a strong promoter, an inducible promoter, a stress-inducible promoter or a tissue-specific promoter.

6. A transgenic plant cell, plant or a part thereof according to claim 4 wherein said regulatory sequence is the CaMV35S promoter.

7. A transgenic plant cell, plant or a part thereof according to claim 4 wherein said regulatory sequence is a tissue-specific promoter.

8. A transgenic plant cell, plant or a part thereof according to claim 7 wherein said promoter is a seed or seedling-specific promoter.

9. A transgenic plant cell, plant or a part thereof according to claim 2 wherein said plant is a seedling and the transition from etioplasts to chloroplasts is accelerated or wherein said plant part is a seed and the transition to amyloplasts is accelerated.

10. A transgenic plant cell, plant or a part thereof according to claim 1 wherein the expression of a nucleic acid comprising SEQ ID NO. 1 or 2 and encoding a SP1 peptide comprising SEQ ID NO. 3, a functional homologue or variant thereof is inactivated, repressed or down-regulated.

11. A transgenic plant cell, plant or a part thereof according to claim 10 wherein plastid development is delayed.

12. A transgenic plant cell, plant or a part thereof according to a claim 10 wherein expression of the endogenous SP1 nucleic acid comprising SEQ ID NO, 1 or 2 encoding a SP1 peptide comprising SEQ ID NO, 3, a functional homologue or variant thereof is silenced.

13. A transgenic plant cell, plant or a part thereof according to claim 12 wherein the transition from chloroplast to gerontoplast is delayed.

14. A transgenic plant cell, plant or a part thereof according to claim 10 wherein said plant part is green tissue.

15. A transgenic plant cell, plant or a part thereof according to claim 1 characterised in that the activity of a SP1 peptide is altered and said plant expresses a nucleic acid comprising a mutant SEQ ID NO. 1 or 2 and encoding a mutant SP1 peptide, a functional homologue or variant thereof which carries a mutation in the RING domain.

16. A transgenic plant cell, plant or a part thereof according to claim 15 wherein the RING domain is as defined in SEQ ID NO. 4 or a domain that has at least 95 homology to SEQ ID NO. 4.

17. A transgenic plant cell, plant or a part hereof according to claim 15 wherein said mutation is a substitution, deletion or insertion.

18. A transgenic plant cell, plant or a part thereof according to claim 17 wherein said mutation is a substitution or deletion of a conserved C or H residue in the RING domain.

19. A transgenic plant cell, plant or a part thereof according to claim 18 wherein said mutation is at a position corresponding to 0330 in A. thaliana SP1 or at an equivalent position in an SP homologue.

20. A transgenic plant cell, plant or a part thereof according to claim 1 wherein said functional homologue has at least 70%, 71%, 72%, 73%, 74%, 75% 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the amino acid represented by SEQ ID NO:3.

21. A transgenic plant cell, plant or a part thereof according to claim 1 wherein said functional homologue is as shown in SEQ ID No. 9, 31, 32, 33, 34, 35, 36, 37, 38, 39, 40, 41, 42, 43, 44, 45, 46, 47, 48, 49, 50, 51, 52, 53, 54, 55 or 56.

22. A transgenic plant cell, plant or a part thereof according to claim 1 wherein said functional homologue comprises a RING domain as defined in SEQ ID NO. 4 or a domain with at least 95% overall sequence identity thereto.

21. A transgenic plant cell, plant or a part thereof according to claim 1 wherein said homologue is SP1 from maize, rice, wheat, oilseed rape, sorghum, soybean, potato, tomato, grape, barley, pea, bean, field bean, lettuce, cotton, sugar cane, sugar beet, broccoli or other vegetable brassicas or poplar.

24. A plant according to claim 1 wherein said plant is a monocot or dicot plant.

25. A plant according to claim 1 wherein said plant is a crop plant or biofuel plant.

26. A plant according to claim 25 wherein said crop plant is selected from maize, rice, wheat, oilseed rape, sorghum, soybean, potato, tomato, grape, barley, pea, bean, field bean, lettuce, cotton, sugar cane, sugar beet, broccoli or other vegetable brassicas or poplar.

27. A product derived from a plant as defined in claim 1 or from a part thereof.

28. A vector comprising a SP1 nucleic acid as defined in SEQ ID NO. 1, 2, 7, 8, 10 or 11, a functional variant or homologue thereof.

29. A vector according to claim 27 further comprising a regulatory sequence which directs expression of the nucleic acid.

30. A host cell comprising a vector according to claim 28.

31. A method for altering plastid development in a transgenic plant cell, plant or a part thereof comprising
a) altering the expression of a nucleic acid comprising SEQ ID NO. 1 or 2 and encoding a SP1 peptide comprising SEQ ID NO. 3, a functional homologue or variant thereof or
b) altering the activity of a SP1 peptide comprising SEQ ID NO. 3 comprising expressing in a plant a nucleic acid comprising a mutant SEQ ID NO. 1 or 2 and encoding a mutant SP1 peptide, a functional homologue or variant thereof which carries a mutation in the RING domain.

32. A method according to claim 31 comprising introducing and expressing in a plant a nucleic acid construct comprising SEQ ID NO. 1 or 2 and encoding a SP1 peptide comprising SEQ ID NO. 3, a functional homologue or variant thereof.

33. A method according to claim 32 wherein chloroplast and/or amyloplast development is accelerated.

34. A method according to claim 32 wherein said construct further comprises a regulatory sequence.

35. A method according to claim 34 wherein said regulatory sequence is a constitutive promoter, a strong promoter, an inducible promoter, a stress-inducible promoter or a tissue-specific promoter.

36. A method according to claim 35 wherein said regulatory sequence is a tissue-specific promoter.

37. A method according to claim 36 wherein said promoter is a seed or seedling-specific promoter.

38. A method according to claim 33 wherein said plant is a seedling and the transition from etioplasts to chloroplasts is accelerated.

39. A method according to claim 32 comprising inactivating, repressing or down-regulating the expression of a nucleic acid comprising SEQ ID NO. 1 or 2 and encoding a SP1 peptide comprising SEQ ID NO. 3, a functional homologue or variant thereof.

40. A method according to claim 39 wherein plastid development is delayed.

41. A method according to a claim 39 wherein expression of the endogenous SP1 nucleic acid comprising SEQ ID NO. 1 or 2 and encoding a SP1 peptide comprising SEQ ID NO. 3, a functional homologue or variant thereof is silenced.

42. A method according to claim 41 wherein the transition from chloroplast to gerontoplast is delayed.

43. A method according to claim 39 wherein said plant part is green tissue.

44. A method according to claim 31 comprising altering the activity of a SP1 peptide comprising SEQ ID NO. 3, a functional homologue or variant thereof is altered comprising expressing in a plant a nucleic acid comprising a mutant SEQ ID NO. 1 or 2 and encoding a mutant SP1 peptide, a functional homologue or variant thereof which carries a mutation in the RING domain.

45. A method according to claim 44 introducing and expressing in a plant a nucleic acid comprising a mutant SEQ ID NO. 1 or 2 and encoding a mutant SP1 peptide, a functional homologue or variant thereof which carries a mutation in the RING domain.

46. A method according to claim 44 wherein the RING domain is as defined in SEQ ID NO. 4 or a domain with at least 95% overall sequence identity thereto.

47. A method according to claim 44 wherein said mutation is a substitution, deletion or insertion.

48. A method according to claim 47 wherein said mutation is a substitution or deletion of a conserved C or H residue in the RING domain.

49. A method according to claim 47 wherein said mutation is at a position corresponding to C330 in A. thaliana SP1 or at an equivalent position in an SP1 homologue.

50. A method for delaying green tissue senescence comprising inactivating, repressing or down-regulating the expression of a nucleic acid comprising SEQ ID NO. 1 and encoding a SP1 peptide comprising SEQ ID NO. 3, a functional homolog or variant thereof.

51. A method for increasing grain/seed size and/or starch content comprising introducing and expressing in a plant a nucleic acid construct comprising SEQ ID NO. 1 or 2 and encoding a SRI peptide comprising SEQ ID NO. 3, a functional homologue or variant thereof.

52. A method for increasing yield of a plant comprising increasing, inactivating, repressing or down-regulating the expression of a nucleic acid comprising SEQ ID NO. 1 or 2 and encoding a SP1 peptide comprising SEQ ID NO. 3, a functional homologue or variant thereof or introducing and expressing in a plant a nucleic acid comprising a mutant SEQ ID NO. 1 or 2 and encoding a mutant SP1 peptide, a functional homologue or variant thereof which carries a mutation in the RING domain.

53. A method for improving seedling emergence, growth and survival comprising introducing and expressing in a plant a nucleic acid construct comprising SEQ ID NO. 1 or 2 and encoding a SRI peptide comprising SEQ ID NO. 3, a functional homologue or variant thereof.

54. A method for altering fruit ripening in a transgenic plant cell, plant or a part thereof comprising
a) altering the expression of a nucleic acid comprising SEQ ID NO. 1 or 2 and encoding a SP1 peptide comprising SEQ ID NO. 3, a functional homologue or variant thereof or
b) altering the activity of a SP1 peptide comprising SEQ ID NO. 3, a functional homologue or variant thereof.

55. A method for making a transgenic plant with altered plastid development comprising
a) altering the expression of a nucleic acid comprising SEQ ID NO. 1 or 2 and encoding a SP1 peptide comprising SEQ ID NO, 2, a functional homologue or variant thereof or
b) altering the activity of a SP1 peptide comprising SEQ ID NO. 2, a functional homologue or variant thereof.

56. A method for increasing stress tolerance to one or more of salinity, osmotic stress and/or oxidative stress in a plant cell, plant or part thereof comprising introducing and expressing in said plant cell, plant or part thereof a nucleic acid construct comprising a nucleic acid comprising SEQ ID NO. 1 or 2 and encoding a SP1 peptide comprising SEQ ID NO. 3, a functional homologue or variant thereof.
